(19) United States
(12) Patent Application Publication
(10) Pub. No.: US 2008/0261910 A1
(43) Pub. Date: Oct. 23, 2008

(54) Diagnostic Methods Based on Polymorphisms of Glucosyltransferase-Like Protein

(75) Inventors: Angela Flannery, Cheshire (GB); Rose Maciewicz, Cheshire (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 12/067,725

(22) PCT Filed: Sep. 20, 2006
(86) PCT No.: PCT/GB06/03468

§ 371 Date: Mar. 21, 2008

(30) Foreign Application Priority Data
Sep. 23, 2005 (GB) 0519376.8

(51) Int. Cl. A61K 31/70; C12Q 1/68; G01N 33/53; A61P 19/00; G01N 33/569

(52) U.S. Cl. 514/44; 435/6; 435/7.1; 435/7.21

(57) ABSTRACT

The present invention arises from the identification of an association between the gene encoding glucosyltransferase-like protein (GT) and osteoarthritis (OA). It therefore relates to diagnostic techniques for determining a patient's susceptibility to develop OA by detecting all or part of the GT gene, its precursors or products (mRNA, cDNA, genomic DNA, or protein). In particular, the invention relates to methods and materials for analysing allelic variation in the GT gene, and to the use of GT polymorphisms in the identification of an individuals' risk to develop OA. The invention is also directed to methods for identifying modulators of OA, which modulate the GT gene or its encoded protein.

### TECHNICAL FIELD

The inventors have discovered a gene linked to susceptibility to osteoarthritis (OA) using linkage and association analysis. The gene is referred to herein as GT. Thus, the present invention identifies a role for GT in OA. The present invention therefore relates to diagnostic techniques for the detection of OA, and for determining a patient's susceptibility to develop OA by detecting all or part of this gene, its precursors or products (mRNA, cDNA, genomic DNA, or protein). The invention also relates to polymorphisms within the gene and to methods for their detection. In particular, the invention relates to methods and materials for analysing allelic variation in the GT gene, and to the use of GT polymorphisms in the identification of an individuals' risk to develop OA.

The polymorphisms of the invention also allow patient stratification. The sub-groups of individuals identified as having increased or decreased risk of developing OA can be used, inter alia, for targeted clinical trial programs and possibly also pharmacogenetic therapies. The invention is also directed to methods for identifying modulators of osteoarthritis, which modulators, such as chemical compounds, antisense molecules and antibodies modulate the gene identified. The invention also relates to single nucleotide polymorphisms (SNPs), which have been found within the gene and to methods for their detection.

### BACKGROUND TO INVENTION

Osteoarthritic diseases are a result of both mechanical and biologic events that destabilize the normal coupling of degradation and synthesis of articular cartilage chondrocytes and extracellular matrix, and subchondral bone (Creamer P. and Hochberg M. C., Lancet, 350, 503-508, 1997; Jones A. and Doherty M., Br. Med. J., 310, 457-460, 1995). Although they may be initiated by multiple factors, including genetic, developmental, environmental factors such as metabolic, and traumatic, osteoarthritis diseases involve all of the tissues of the diarthrodial joint. Ultimately, osteoarthritis diseases are manifested by morphologic, biochemical, molecular, and biomechanical changes of both cells and matrix which lead to a softening, fibrillation, ulceration, loss of articular cartilage, sclerosis and eburnation of subchondral bone, osteophytes, and subchondral cysts. Other tissues besides cartilage, such as tendon, ligament, muscle and meniscus can also contribute to disease initiation and progression. When clinically evident, osteoarthritis diseases are characterized by joint pain, tenderness, limitation of movement, crepitus, occasional effusion, and variable degrees of inflammation mainly without overt systemic effects (Kuettner and Goldberg. Osteoarthritic disorders. Rosemont: American Academy of Orthopaedic Surgeons, 1995).

Most epidemiological studies have defined cases of osteoarthritis based on the presence of typical radiographic features. In populations of white North Americans and Northern Europeans, about one-third of adults aged 25-74 years have features of radiographic osteoarthritis involving at least one peripheral joint group: the most common sites are the hands, followed by feet, knees, and hips (T D Spector and M C Hochberg, Ann Rheum Dis 53:43-46, 1994). However, OA in the knees and hips are the more clinically important.

Age is the strongest determinant of osteoarthritis with prevalence rates for all joints rising with increasing age. Incidence rates also rise with age but there is some evidence that this reaches a plateau in the seventh decade. The mechanism by which age predisposes to osteoarthritis is unclear. Obesity has been strongly linked to osteoarthritis of the knee and, to a lesser extent, the hip, in cross-sectional and prospective studies (Hochberg M C, Lethbridge-Cejku M. Hamerman D, ed. Osteoarthritis: public health implications for an aging population. Baltimore: Johns Hopkins University Press, 1997:169-86).

Current treatment of osteoarthritis is purely to control symptoms because as yet there are no disease-modifying osteoarthritis drugs. The principal measure of treatment efficacy is traditionally pain. The drug of choice is currently paracetamol to which a proportion of patients do not respond but appear to derive benefit from NSAIDs (non-steroidal anti-inflammatory drugs). Topical creams, either a NSAID or capsaicin, can be helpful as either monotherapy or when added to oral analgesics, especially if only a few joints are involved.

Intra-articular steroids are widely used in osteoarthritis, particularly for the knee. There is some evidence to support their efficacy versus pain relief, but only for 1-3 weeks; triamcinolone hexacetonide is the most efficacious preparation. Intra-articular hyaluronic acid has also been shown to be effective in modulating pain in patients with knee osteoarthritis; this therapy requires weekly injections for 3-5 weeks and is more efficacious than a single injection of intra-articular steroids.

Genetic components have been demonstrated to exist in osteoarthritis by heritability estimates in twins and by relative risk estimates in siblings. For hand and knee OA, the genetic influence has been calculated at between 39-65% in twins. In a separate study of hand and disc OA the estimates were 56% and 75% respectively. In severe hip OA requiring total hip replacement (THR), the sibling relative risk compared to the population at large has been calculated at 5.6 and this may be an underestimate, since the prevalence of OA in the general population is high. A recent study of female twin pairs showed significant heritability for radiographic features of osteoarthritis at the knee and hip (Spector et al, BMJ, 312 (1996), pp. 940-944). Although mutations in the gene for type II collagen (COL2A1) have been associated with early polyarticular osteoarthritis with mild chondrodysplasia (Knowlton et al New Eng. J. Med. 322: 526-530, 1990), it is unlikely that there is a single gene that fully explains the genetic contribution to osteoarthritis. However, as OA demonstrates considerable clinical heterogeneity there may be specific gene(s) for specific sub classes of OA (Peach et al Trends Mol. Med. 11 (4): 186-191, 2005).

There is therefore a desire to identify genes with a significant association to the development of OA. This may enable the development of novel therapies for OA by screening for compounds and other entities, such as antibodies, which modulate the activity of the proteins encoded by the associated genes, or modulate their interaction with other proteins or molecules. Knowledge of the sequence of the associated genes may also enable the development of novel antigene methods to modulate the expression of the associated gene and may also enable the development of novel gene therapy techniques to treat OA. The discovery of associated genes may also assist in developing novel methods for diagnosing OA via (i) analysis of the pattern of genotypes of associated single nucleotide polymorphisms (SNPs), (ii) measuring the levels of the translated mRNA present in affected tissue and (iii) measuring the levels of the protein in affected tissue. It is possible that the diagnosis of OA, or the prediction of predisposition to OA, by these methods may be achieved in patients who do not yet display the classical symptoms of the disease. Such determination of susceptibility to OA or the early detection of disease development may lead to earlier clinical intervention than is currently possible and may lead to more effective treatment of the disease. Such techniques may also allow patient stratification, which may inter alia, be useful in selecting patients for clinical trials.

International Patent publication Number: WO 00/20632 identifies a 20cM region on chromosome 11 between microsatellite markers D11S4046 and D11S1320 that contains a possible susceptibility locus for OA. This region was identified following a two-stage non-parametric linkage analysis. This revealed three microsatellites on chromosome 11 for which there was evidence of linkage. Finer mapping in and around these microsatellites provided enhanced evidence for linkage and enabled linked regions to be defined. Linkage disequilibrium analysis identified excess transmission of the 258 bp allele of marker D11S937 (allele 11 as listed in the GDB (Genome Database)) in the unstratified data (p<0.001). This data indicated that the OA susceptibility locus on 11q is close to D11S937 and that the 258 bp allele may be present on a predisposing ancestral chromosome. There is however, no disclosure of an association to OA for any single gene nor is any indication as to where such a gene is likely to be located within the 20 cM region disclosed.

The present invention is based on our discovery of an association with OA for a single gene termed glucosyltransferase-like protein (GT or GTOA), which maps to a 2.8 cM region within 11q13-14, which incorporates D11S937 at 11q13.5.

This gene is designated in the public databases as a glucosyl transferase by homology with known yeast enzymes, which add a glucose moiety during protein glycosylation. Yeast complementation experiments have been carried out by the inventors in yeast Saccharomyces cerevisiae strains deficient in the activity of four different glucosyl transferases (namely ALG6, ALG8, ALG6, ALG10) and these experiments have shown that GT is the functional orthologue of ALG8. Glucosyl transferase activity, along with the activity of a number of other glycosyl transferases, is upregulated in chondrocytes from osteoarthritic cartilage. A glucosyl transferase activity is involved in glycosylation of hydroxyproline residues in collagen; glycosylation is a factor in controlling fibrillar morphology (Richard M, Vignon E, Peschard M J, Broquet P, Carret J P and Louisot P (1990) Biochem. Int. 22:535-542). No association to OA has previously been described for this glucosyltransferase-like gene.

The GT protein (also referred to herein as GTOA) is referenced in the databases as AJ224875. The predicted amino acid sequence of this protein is shown in SEQ ID No:2. There is evidence of alternative splicing from the mRNA template, which might give rise to alternative amino acid sequences. In addition there is a nucleotide variant which gives rise to an amino acid change. The predicted amino acid sequence arising from this variant is shown in SEQ ID No.3. The genomic sequence is disclosed as SEQ ID NO: 1.

### SUMMARY OF THE INVENTION

The present inventors have identified an osteoarthritis disease association with a gene on chromosome 11, and have identified polymorphisms within this gene. The present application therefore provides direct evidence for a role for GTOA in osteoarthritis. The GTOA gene, mRNA and protein sequences derived therefrom may therefore be used as diagnostic or prognostic markers of OA, and can be used to design specific probes, or to generate antibodies, capable of detecting the presence of polymorphisms of the gene or mRNA, or of measuring the levels of the mRNA or encoded protein present in a test sample, such as a body fluid or cell sample. In addition the gene and protein encoded thereby is a potential target for therapeutic intervention in osteoarthritis disease, for instance in the development of antisense nucleic acid targeted to the mRNA; or more widely in the identification or development of chemical or hormonal therapeutic agents. The person skilled in the art is capable of devising screening assays to identify compounds (chemical or biological) that modulate (e.g. activate or inhibit) the identified gene, which compounds may prove useful as therapeutic agents in treating or preventing osteoarthritis and arthropathies in general.

### DETAILED DESCRIPTION OF THE INVENTION

The following definitions apply to the present application.

“Allele” refers to a particular form of a genetic locus, distinguished from other forms by its particular nucleotide or amino acid sequence.

“Expression” refers to the transcription of a gene's DNA template to produce the corresponding mRNA and translation of this mRNA to produce the corresponding gene product (i.e., a peptide, polypeptide, or protein). The term “activates gene expression” refers to inducing or increasing the transcription of a gene in response to a treatment where such induction or increase is compared to the amount of gene expression in the absence of said treatment. Similarly, the terms “decreases gene expression” or “down-regulates gene expression” refers to inhibiting or blocking the transcription of a gene in response to a treatment and where such decrease or down-regulation is compared to the amount of gene expression in the absence of said treatment.

“Genotype” is an unfazed 5′ to 3′ sequence of nucleotide pair(s) found at one or more polymorphic sites in a locus on a pair of homologous chromosomes in an individual.

“Isolated” nucleic acid, as referred to herein, refers to material removed from its original environment (for example, the natural environment in which it occurs in nature), and thus is altered by the hand of man from its natural state. For example, an isolated polynucleotide could be part of a vector or a composition of matter, or could be contained within a cell, and still be “isolated” because that vector, composition of matter, or particular cell is not the original environment of the polynucleotide.

“Locus” refers to a location on a chromosome or DNA molecule corresponding to a gene or a physical or phenotypic feature.

“Polymorphic site” is a position within a locus at which at least two alternative sequences are found in a population. “Polymorphism” refers to the sequence variation observed in an individual at a polymorphic site. Polymorphisms include nucleotide substitutions, insertions, deletions and microsatellites and may, but need not, result in detectable differences in gene expression or protein function.

Thus according to a first aspect of the invention there is provided a method for identifying a compound capable of modulating the action of the GT protein which method comprises subjecting one or more test compounds to a screen comprising a polypeptide containing the amino acid sequence shown in SEQ ID NO: 2, or a homologue thereof or a fragment of either.

The term “fragment” as used herein refers to a subsequence of the full length sequence that comprises at least 25, preferably at least 50, more preferably at least 100 consecutive amino acids of the sequence depicted in SEQ ID NO: 2, preferably the fragment is a polypeptide that is the GT protein with either or both C-terminal and N-terminal truncations.

It is understood that the polypeptide for use in the invention may be both a fragment and a homologue of the GT protein.

In a preferred embodiment, the screening methods of the invention are carried out using a polypeptide comprising an amino acid sequence as depicted in SEQ ID NO: 2, or a sequence possessing, in increasing order of preference, at least 80%, 85%, 90%, 95%, 97%, 98% and 99% amino acid sequence identity thereto. Such variants are herein referred to as “homologues”. In another embodiment the screen is conducted with a splice variant of the GT protein. Suitable homologues for use in the invention include those whose nucleic acids hybridise to a nucleic acid sequence encoding the polypeptide depicted in SEQ ID NO: 2 under stringent hybridisation conditions, which homologues retain a function of the GT protein.

The sequence identity between two sequences can be determined by pair-wise computer alignment analysis, using programs such as, BestFit, Gap or FrameAlign. The preferred alignment tool is BestFit. In practise, when searching for similar/identical sequences to the query search, from within a sequence database, it is generally necessary to perform an initial identification of similar sequences using suitable software such as Blast, Blast2, NCBI Blast2, WashU Blast2, FastA, Fasta3 and PILEUP, and a scoring matrix such as Blosum 62. Such software packages endeavour to closely approximate the “gold-standard,” alignment algorithm of Smith-Waterman. Thus, the preferred software/search engine programme for use in assessing similarity, i.e how two primary polypeptide sequences line up is Smith-Waterman. Identity refers to direct matches, similarity allows for conservative substitutions.

Allelic variants or versions of the GT protein may exist within the human population, particularly between distinct ethnic groups. A further aspect of the invention involves the selection and use of the appropriate version of the GT protein to be included in screens so as to discover compounds capable of altering the activity of said GT version in vivo. Investigators may wish to screen their compounds against the most prevalent version of the GT protein and also against the less frequent versions of the GT protein in order to detect any differential pharmacological activity between the various versions of the target. A further aspect of the invention is the screening of various ethnic based populations to measure the allele frequencies of the single nucleotide polymorphisms (mutations) in the GT gene within said populations. This information may be of value in estimating the efficacy of new compounds capable of altering the activity of GT within these populations and in particular in estimating the proportion of the population, which may not respond to the therapy.

According to another aspect of the invention there is provided a method for identifying a compound capable of inhibiting/antagonizing the activity of GT comprising bringing into contact:

(i) a test compound;

(ii) a cell membrane preparation comprising GT

(iii) an oligosaccharide, capable of allowing glucose addition by GT; and,

(iv) glucose; and,

measuring the effect that the test compound has on the ability of GT to add glucose to the oligosaccharide.

According to a particular embodiment, a test compound that impedes or blocks glucose addition to the oligosaccharide is one that inhibits/antagonizes the activity of GT.

The cell membrane preparation can be one prepared from any eukaryotic organism. Suitable organisms are yeast or mammalian, e.g. human, rodent, simian. The only requirement is that the cell membrane preparation contains or comprises active GT enzyme. In a particular embodiment the cell is capable of expressing the GT enzyme. The GT expressed from the eukaryotic cell can be homologous or heterologous. Preferably, the GT enzyme is of human origin. In a particular embodiment the cell membrane preparation is from yeast cells recombinantly engineered to express human GT. In another embodiment it is derived from mammalian cells recombinantly engineered to express human GT. In another embodiment the cell is of mammalian origin, which expresses GT endogenously (i.e. it has not been engineered to express heterologous GT). The term “heterologous” as used herein refers to any DRR gene transfected into a cell, i.e., the term refers to any non-endogenous DRR.

There are various cell membrane preparative methods available to the person skilled in the art. Representative examples are described in Radominska-Pandya et al. (Methods Enzymol.400:116-47, 2005) or Shimma et al. (Appl Environmental Microbiol. Aug. 25, 2006).

The oligosaccharide can be naturally occurring (e.g. derived from any prokaryote or eukaryote cell), either integral with endoplasmic reticulum (ER) membranes or purified away from ER membranes, plus or minus a dolichol moiety (e.g. Kelleher et al, Glycobiology. 11(4):321-33, 2001); or synthetic (e.g. Weiss & Unverzagt. Angew Chem Int Ed Engl. 42(35):4261-3, Sep. 15, 2003). The oligosaccharide simply serves as the substrate onto which GT can add a glucose moiety. In a particular embodiment, the oligosaccharide is whole (i.e. GlcNac2, Man9). In another it possesses the dolichol moiety.

The oligosaccharide can be free or lipid-linked; and, either free (unbound) or bound to a substrate, such as a nylon membrane, plate, bead etc. The oligosaccharide can also be, or be derived from a cell membrane preparation containing dolichol-linked oligosaccharides.

The glucose in the assay system is preferably tagged or labeled so as to be capable of being detected following addition to the oligosaccharide. Any suitable label or tag can be used. Representative examples include non- or radioactive (e.g. P³², S³⁵, I¹²⁵H³) or non-radioactive label (e.g. fluorophore), biotin/avidin, photo-affinity probes (e.g. [β-³²P]5N₃UDP-Glc (Drake et al., J. Biol. Chem. 267:11360-11365, 1992)) as well as protein (e.g. antibody tagging).

Binding of glucose to oligosaccharide can be measured by any suitable method known to the person skilled in the art. These include: filter-binding assay, Scintillation Proximity Assay (SPA) technology, ELISA, Lectin binding. Indeed, any method that can capture or detect a protein/sugar tagged product.

Candidate compounds may be identified from a variety of sources, for example, cells, cell-free preparations, chemical libraries, peptide and gene libraries, and natural product mixtures. Chemical libraries include combinatorial chemistry libraries and, in particular, a combinatorial chemical library comprising compounds that interact with GPCRs. Such antagonists or inhibitors so-identified may be natural or modified substrates, ligands, receptors, enzymes, antibodies (as described above) etc., as the case may be, of the GT protein; or may be structural or functional mimetics thereof (see Coligan et al., Current Protocols in Immunology 1(2):Chapter 5 (1991)).

Techniques such as analytical centrifugation affinity binding studies involving chromatography or electrophoresis can be used to detect molecules, which interact directly with GT. Other techniques that allow the identification of protein-protein interactions include immunoprecipitation and yeast two hybrid studies.

Compounds having inhibitory, activating, or modulating activity can be identified using in vitro and in vivo assays for GT activity and/or expression, e.g., ligands, agonists, antagonists, and their homologs and mimetics.

Further aspects of the invention arise from the identification of single base change polymorphisms (mutations) in GT as outlined in Table 2.

Polymorphism refers to the occurrence of two or more genetically determined alternative alleles or sequences within a population. A polymorphic marker is the site at which divergence occurs. Preferably markers have at least two alleles, each occurring at frequency of greater than 1%, and more preferably at least 10%, 15%, 20%, 30% or more of a selected population.

Single nucleotide polymorphisms (SNP) are generally, as the name implies, single nucleotide or point variations that exist in the nucleic acid sequence of some members of a species. Such polymorphic variations within the species are generally regarded to be the result of spontaneous mutation throughout evolution. The mutated and normal sequences co-exist within the species' population sometimes in a stable or quasi-stable equilibrium. At other times the mutation may confer some advantage to the species and with time may be incorporated into the genomes of all or a majority of members of the species.

Some SNPs alter the protein coding sequences, in which case, one of the polymorphic protein forms may possess a different amino acid which may give rise to the expression of a variant protein and, potentially, a genetic disease. These changes in function may be mediated by several mechanisms including, but not limited to, alterations in protein folding, alterations in ligand and substrate binding affinity and alterations in membrane binding affinity and may lead to gain of activity or loss of activity for the protein in vivo. Such alterations in the activity of the protein in vivo may be of clinical significance in the development of OA. Alteration to the amino acid sequence of the protein may also affect the efficacy of drug therapy for OA by altering the specificity between protein and compounds selected by screening to modulate its activity. Other SNPs occur in regulatory regions of the gene, in promoters, splice donor or acceptor sites, in 3'UTRs or 5'UTRs. Variants in such sites may alter the function and/or the expression of the gene in a similar way to, but not limited to, protein coding variants. Thus compounds selected by screening may have different efficacies in modulating the activity of protein in different individuals according to the versions of the gene that they carry. In particular an individual who is homozygous for a less common variant of the gene may not respond well to a therapy developed by screening compounds against the dominant variant.

The use of knowledge of polymorphisms to help identify patients most suited to therapy with particular pharmaceutical agents is often termed “pharmacogenetics”. Pharmacogenetics can also be used in pharmaceutical research to assist the drug selection process. Polymorphisms are used in mapping the human genome and to elucidate the genetic component of diseases. The reader is directed to the following references for background details on pharmacogenetics and other uses of polymorphism detection: Linder et al. Clinical Chemistry, 43:254, 1997; Marshall. Nature Biotechnology. 15:1249, 1997; International Patent Application WO 97/40462, Spectra Biomedical; and Schafer et al, Nature Biotechnology. 16:33, 1998.

A haplotype is a set of alleles found at linked polymorphic sites (such as within a gene) on a single (paternal or maternal) chromosome. If recombination within the gene is random, there may be as many as 2ⁿ haplotypes, where 2 is the number of alleles at each SNP and n is the number of SNPs. One approach to identifying mutations or polymorphisms, which are correlated with clinical response is to carry out an association study using all the haplotypes that can be identified in the population of interest. The frequency of each haplotype is limited by the frequency of its rarest allele, so that SNPs with low frequency alleles are particularly useful as markers of low frequency haplotypes. As particular mutations or polymorphisms associated with certain clinical features, such as adverse or abnormal events, are likely to be of low frequency within the population, low frequency SNPs may be particularly useful in identifying these mutations (for examples see: Linkage disequilibrium at the cystathionine beta synthase (CBS) locus and the association between genetic variation at the CBS locus and plasma levels of homocysteine (De Stefano et al., Ann Hum Genet. 62:481-90, 1998; and, Keightley et al., Blood 93:4277-83, 1999).

Clinical trials have shown that patient response to treatment with pharmaceuticals is often heterogeneous. Thus there is a need for improved approaches to pharmaceutical agent design and therapy, as well as clinical trial design (including patient selection). The ability to stratify patients into groups based on their likely response to the drug (e.g. in terms of safety or efficacy) would be extremely useful in the quest to identify appropriate drugs of therapeutic value.

Point mutations in polypeptides will be referred to as follows: natural amino acid (using 1 or 3 letter nomenclature), position, new amino acid. For (a hypothetical) example “D25K” or “Asp25Lys” means that at position 25 an aspartic acid (D) has been changed to lysine (K). Multiple mutations in one polypeptide will be shown between square brackets with individual mutations separated by commas.

The presence of a particular nucleic acid base at a polymorphism position will be represented by the base following the polymorphism position. For (a hypothetical) example, the presence of adenine at position 300 will be represented as: 300 A. We provide examples of single nucleotide polymorphisms (mutations), which potentially affect the sequence of the GT protein. These are shown in Table 4.

Single nucleotide polymorphisms (mutations) in the promoter and UTR regions may also affect the transcription and expression of the GT gene leading to either increased or decreased levels of expression or to unregulated activity of the GT protein in vivo. Such alterations in the level of expression of the GT protein in vivo may result in a gain or loss of function, which is of clinical significance. Recently, it has been reported that even polymorphisms that do not result in an amino acid change can cause different structural folds of mRNA with potentially different biological functions (Shen et al., Proc Natl Acad Sci USA 96:7871-7876, 1999).

In one embodiment of the invention the screening methods described herein utilise a GT protein variant which is transcribed from the nucleic acid sequence shown in SEQ ID NO:1 and which incorporates one or more of the SNPs identified herein and listed in Table 2.

The polymorphisms (mutations) may also be used as diagnostic markers of predisposition to disease. Genotyping SNPs in populations suffering from OA and in control populations not suffering from OA but matched for factors including, but not limited to, racial ancestry, country of origin, sex, age and body mass index may allow investigators to identify increased risk factors associated with the development of OA disease according to the inheritance of certain SNP genotypes or haplotypes which are more prevalent in populations with OA compared to their incidence in the corresponding control populations. This may enable screening for individuals at increased risk of developing OA by measuring the genotypes and haplotypes of these SNPs within non-symptomatic individuals. We have discovered several SNPs in the GT gene, which may be useful for the diagnosis of increased risk to develop OA. These SNPs are shown in Table 2.

The screening methods according to the invention may be operated using conventional procedures, for example by bringing the test compound or compounds to be screened and an appropriate substrate into contact with the polypeptide, or a cell capable of producing it, or a cell membrane preparation thereof, and determining affinity for the polypeptide in accordance with standard techniques.

Any compound identified in this way may prove useful in the treatment of osteoarthritis in humans and/or other animals. The invention thus extends to a compound selected through its ability to regulate the activity of the GT protein in vivo as primarily determined in a screening assay utilising the polypeptide containing an amino acid sequence shown in SEQ ID NO: 2, or a homologue or fragment thereof, or a gene coding therefore (such as that disclosed in SEQ ID NO: 1) for use in the treatment of a disease in which the over- or under-activity or unregulated activity of the protein is implicated.

According to a further aspect of the invention there is provided a screening assay or method for identifying potential disease modifying anti-OA drugs (DMOAD) comprising contacting an assay system capable of detecting the effect of a test compound against expression level of GT, with a test compound and assessing the change in expression level of GT. In a particular embodiment a potential therapeutic compound for treating an OA disorder is one capable of reducing the expression level of GT.

Compounds that modulate the expression of DNA or RNA of GT polypeptides may be detected by a variety of assay systems. A suitable assay system may be a simple “yes/no” assay to determine whether there is a change in expression of a reporter gene, such as beta-galactosidase, luciferase, green fluorescent protein or others known to the person skilled in the art (reviewed by Naylor, Biochem. Pharmacol. 58:749-57, 1999). The assay system may be made quantitative by comparing the expression or function of a test sample with the levels of expression or function in a standard sample. Systems in which transcription factors are used to stimulate a positive output, such as transcription of a reporter gene, are generally referred to as “one-hybrid systems” (Wang and Reed. Nature 364:121-126, 1993). Using a transcription factor to stimulate a negative output (growth inhibition) may thus be referred to as a “reverse one-hybrid system” (Vidal et al, 1996, supra). Therefore, in an embodiment of the present invention, a reporter gene is placed under the control of the GT promoter.

In a further aspect of the invention we provide a cell or cell line comprising a reporter gene under the control of the GT promoter.

According to another aspect of the present invention there is provided a method of screening for a compound potentially useful for treatment of OA, which comprises assaying the compound for its ability to modulate the activity or amount of GT. Preferably the assay is selected from:

i) measurement of GT activity using a cell line which expresses the GT polypeptide or using purified GT polypeptide; and
ii) measurement of GT transcription or translation in a cell line expressing the GT polypeptide.

In a particular embodiment a potential therapeutic compound for treating an OA disorder is one capable of reducing the activity or amount of GT.

The “GT polypeptide” refers to the GT protein (aka GTOA protein), a homologue thereof, or a fragment of either.

Thus, in a further aspect of the invention, cell cultures expressing the GT polypeptide can be used in a screen for therapeutic agents. Effects of test compounds may be assayed by changes in mRNA or protein of GT. As described below, cells (i.e. mammalian, bacterial etc) can be engineered to express the GT polypeptide.

Thus, according to a further aspect of the invention there is provided a method of testing potential therapeutic agents for the ability to suppress osteoarthritis disorder phenotype comprising contacting a test compound with a cell engineered to express the GT polypeptide; and determining whether said test compound suppressed expression of the GT polypeptide.

We also provide a method for identifying inhibitors of transcription of GT, which method comprises contacting a potential therapeutic agent with a cell or cell line as described above and determining inhibition of GT transcription by the potential therapeutic agent by reference to a lack of or reduction in expression of the reporter gene.

Any convenient test compound or library of test compounds may be used in conjunction with a test assay. Particular test compounds include low molecular weight chemical compounds (preferably with a molecular weight less than 1500 daltons) suitable as pharmaceutical or veterinary agents for human or animal use, or compounds for non-administered use such as cleaning/sterilizing agents or for agricultural use. Test compounds may also be biological in nature, such as antibodies.

According to a further aspect of the invention there is provided a compound identified by a screening method as defined herein.

According to another aspect of the present invention there is provided use of a compound able to modulate the activity or amount of GT in the preparation of a medicament for the treatment of OA. Modulation of the amount of GT by a compound may be brought about for example through altered gene expression level or message stability. Modulation of the activity of GT by a compound may also be brought about for example through compound binding to the GT protein. In one embodiment, modulation of GT comprises use of a compound able to reduce the activity or amount of GT. In another embodiment, modulation of GT comprises use of a compound able to increase the activity or amount of GT.

It will be appreciated that the term ‘for the treatment of OA’, and variations thereon, includes therapeutic and prophylactic (preventative) treatment.

According to another aspect of the present invention there is provided a method of preparing a pharmaceutical composition which comprises:

i) identifying a compound as useful for treatment of OA according to a screening method as described herein; and
ii) mixing the compound or a pharmaceutically acceptable salt thereof with a pharmaceutically acceptable excipient or diluent.

According to a further aspect of the invention there is provided a method of treatment of a patient suffering from OA comprising administration to said patient of an effective amount of a compound identified according to a screening method of the invention or a composition prepared by the method described herein.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intraarticular, or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal track, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxyethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid. Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient, which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient. For further information on Formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients, which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound will naturally vary according to the nature and severity of the conditions, the age and sex of the animal or patient and the route of administration, according to well known principles of medicine.

In using a compound for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred.

Having identified that the GT gene is implicated in OA, this presents many molecular diagnostic opportunities. It is known to persons skilled in the art that clinically significant information may be obtained by the measurement of the levels of nucleic acids, proteins or other analytes that occur within biological samples. When nucleic acids, proteins or other analytes occur in polymorphic form then there may also be diagnostic utility in by identifying which of the various versions of said polymorphic nucleic acids, proteins or other analytes occur within a sample.

An investigator may wish to measure the levels of GT protein or to measure the levels of GT mRNA transcript present in a sample. An investigator may also wish to perform nucleic acid sequence analyses to detect variant nucleotides (SNPs) present within the sample, these analyses may be performed on either the DNA or RNA fraction of the sample and are well known to the person skilled in the art. An investigator may also wish to perform protein sequence analysis either directly by degradation based techniques which are well known in the art or indirectly by molecular recognition techniques including immunoassay or by techniques based on detecting changes in the physical characteristics of the protein such as functional or substrate specificity assays or iso-electric focusing.

According to a further aspect of the invention there is provided a method for diagnosing or prognosing or monitoring OA, comprising testing a biological sample for aberrant levels of GT.

The term “aberrant levels” refers to levels that are outside the normal range. The normal range can be determined by testing many normal tissues or may be determined from a side by side comparison of the test sample with the normal or control sample. For the purposes of this application, aberrant expression refers to a 1.5-fold difference or more in level of nucleic acid in a disease sample compared to control normal. Nucleic acid as used herein refers to both RNA and DNA.

The test sample is conveniently a sample of synovial fluid, blood (including plasma or serum), buccal scrape, urine or other body fluid or tissue obtained from an individual. The invention lies in the identification of the gene identified herein being linked to OA disease prevalence. Accordingly, in part, the invention is directed to any diagnostic method capable of assessing the differential expression level, relative to expression in control tissues, of the GT gene identified herein, either alone or as a panel. In particular, such methods include assessment of mRNA transcript levels and/or protein levels where the presence of aberrant expression levels of the gene indicate the presence of OA or an increased likelihood to develop the disorder.

As noted above, in one embodiment the diagnostic/detection methods of the invention are employed to detect the presence of one or more polymorphisms of GT.

According to another aspect of the present invention there is provided a method for the diagnosis of, or susceptibility to develop, osteoarthritis in a human, which method comprises determining the sequence of the nucleic acid of the human at one or more of positions: 5283, 21037, 21134, 25515, 34419, 59661, 59968 and 73821 . . . 73842 (each according to SEQ ID NO: 1), or a polymorphism in linkage disequilibrium above D'0.9 therewith, and determining the status of the human by reference to polymorphism in or flanking the GT gene.

According to another aspect of the present invention there is provided a method for the diagnosis of a polymorphisms in GT, which method comprises determining the sequence of the nucleic acid of the human at one or more of positions: 5283, 21037, 21134, 25515, 34419, 59661, 59968 and 73821 . . . 73842 (each according to SEQ ID NO: 1), or a polymorphism in linkage disequilibrium above D' 0.9 therewith, and determining the status of the human by reference to polymorphism in or flanking the GT gene.

In a particular embodiment, this method is used to assess the predisposition and/or susceptibility to develop OA in the human (e.g. assessing the risk of developing OA at a future date). The particular alleles that show an association with OA are identified in Table 3.

In particular embodiments, the presence of a C (cytosine) at position 5283 and/or a T (thymine) at position 21037 and/or a T at position 21134 and/or a T at position 25525 and or a T at position 34419 and/or a C at position 59661 and/or a C at position 59968 and/or a deletion of the sequence from positions 73821-73842 (each according to the location in SEQ ID NO: 1) is indicative of an increased risk of developing OA (e.g. predisposition to OA).

It should further be noted that detection of the nucleotide in the complement strand to SEQ ID NO:1 that base-pairs with the nucleotide at a particular position (e.g. position 34419 of SEQ ID NO:1) is of course within the scope of the claimed invention.

According to another aspect of the present invention there is provided a method for genotyping an individual, which method comprises determining the sequence of the nucleic acid of the human at one or more of positions: 5283, 21037, 21134, 25515, 34419, 59661, 59968 and 73821 . . . 73842 (each according to SEQ ID NO: 1), or a polymorphism in linkage disequilibrium above D′ 0.9 therewith, and determining the genotype status of the human by reference to polymorphism in the GT gene. The genotype status of the individual can then be used to gauge the individual's risk of developing OA.

In particular embodiments, these particular aspects are applied to the detection or one or more of the polymorphisms identified in Tables 2 or 3.

The term “diagnosis of a polymorphism” refers to determination of the genetic status of an individual at a polymorphic position (in which the individual may be homozygous or heterozygous at each position).

The term “status” refers to the genetic status of the human as detected by potential sequence variation at defined positions of a polynucleotide or corresponding protein.

Determining the status of the individual can simply be determining the genotype of the individual (i.e. the identity of the allele at the particular polymorphism) and/or translating that information into a clinical significance, (e.g. assigning a likely risk of developing OA).

The polymorphisms of the invention demonstrate significant association to OA. However, the person skilled in the art will appreciate that OA is a polygenic disease and therefore, a diagnostic test consisting solely of a polymorphism (e.g. a SNP), or even a haplotype comprising one or more polymorphisms of the invention will not be diagnostic of disease occurrence for any particular individual. Nevertheless, in line with future developments we envisage that the polymorphisms of the present invention could form part of a panel of markers that in combination will be predictive of disease or disease susceptibility for an individual, within normal clinical standards sufficient to influence clinical practice.

According to another aspect of the invention there is provided a method of determining if an individual is predisposed to OA, the method comprising determining a presence or absence, in a homozygous or heterozygous form, of at least one OA-associated genotype in the GT locus or in neighbouring loci of the individual, said neighbouring loci being in linkage disequilibrium with said GT locus, thereby determining if the individual is predisposed to OA.

According to another aspect of the invention there is provided a method for assessing the predisposition and/or susceptibility of an individual to OA, which method comprises:
(i) providing a nucleic acid sample that has been removed from the individual;
(ii) determining the identity of one or more nucleotides at a polymorphic site in the GT gene of the individual; and
   determining the status of the individual by reference to said one or more nucleotides.

In another aspect of the invention there is provided a method for the diagnosis of OA or determining susceptibility to develop OA, which method comprises:

i) obtaining a protein or nucleic acid containing sample from an individual;
ii) detecting the presence or absence of a variant GT on the basis of the presence of a polymorphic amino acid within the GT protein, or a polymorphic base within the GT gene sequence; and,
iii) determining the status of the human by reference to the presence or absence of a polymorphism in GT.

It will be apparent to the person skilled in the art that there are a large number of analytical procedures, which may be used to detect the presence or absence of variant nucleotides at one or more polymorphic positions of the invention. In general, the detection of allelic variation requires a mutation discrimination technique, optionally an amplification reaction and optionally a signal generation system. List 1 lists a number of mutation detection techniques, some based on the polymerase chain reaction (PCR). These may be used in combination with a number of signal generation systems, a selection of which is listed in List 2. Further amplification techniques are listed in List 3. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

TABLE 1
Abbreviations:

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| CMC | Chemical mismatch cleavage |
| Bp | base pair |
| COPS | Competitive oligonucleotide priming system |
| DGGE | Denaturing gradient gel electrophoresis |
| FRET | Fluorescence resonance energy transfer |
| LCR | Ligase chain reaction |
| MASDA | Multiple allele specific diagnostic assay |
| NASBA | Nucleic acid sequence based amplification |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SERRS | Surface enhanced raman resonance spectroscopy |
| SNP | Single nucleotide polymorphism |
| SSCP | Single-strand conformation polymorphism analysis |
| SSR | Self sustained replication |
| TGGE | Temperature gradient gel electrophoresis |
| 3′ UTR | 3′ untranslated region |

### List 1—Mutation Detection Techniques

General: DNA sequencing, Sequencing by hybridisation
Scanning: PTT*, SSCP, DGGE, TGGE, Cleavase, Heteroduplex analysis, CMC, Enzymatic mismatch cleavage
Note: not useful for detection of promoter polymorphisms.
   Hybridisation Based Solid phase hybridisation: Dot blots, MASDA, Reverse dot blots, Oligonucleotide arrays (DNA Chips)
   Solution phase hybridisation: Taqman™—U.S. Pat. No. 5,210,015 & U.S. Pat. No. 5,487,972 (Hoffmann-La Roche), Molecular Beacons—Tyagi et al (1996), Nature Biotechnology, 14, 303; WO 95/13399 (Public Health Inst., New York)
   Extension Based: ARMS™—allele specific amplification (as described in European patent No. EP-B-332435 and U.S. Pat. No. 5,595,890), ALEX™—European Patent No. EP 332435 B1 (Zeneca Limited), COPS—Gibbs et al (1989), Nucleic Acids Research, 17, 2347.

### Incorporation Based Mini-sequencing, APEX

Restriction Enzyme Based: RFLP, Restriction site generating PCR

### Ligation Based: OLA

Other: Invader assay, Hybridisation protection assay

### List 2—Signal Generation or Detection Systems

Fluorescence: FRET, Fluorescence quenching, Fluorescence polarisation—United Kingdom Patent No. 2228998 (Zeneca Limited)
Other: Chemiluminescence, Electrochemiluminescence, Raman, Radioactivity, Colorimetric, Mass spectrometry, SERRS—WO 97/05280 (University of Strathclyde).

### List 3—Further Amplification Methods

SSR, NASBA, LCR, SDA, b-DNA
Preferred mutation detection techniques include ARMS™-allele specific amplification, Taqman™, Mini sequencing, sequencing, RFLP, ALEX™, OLA, restriction site based PCR and FRET techniques.
Particularly preferred methods include ARMS™—allele specific amplification, OLA and RFLP based methods. ARMS™-allele specific amplification is an especially preferred method.

ARMS™-allele specific amplification (described in European patent No. EP-B-332435, U.S. Pat. No. 5,595,890 and Newton et al. (Nucleic Acids Research, Vol. 17, p. 2503; 1989)), relies on the complementarity of the 3′ terminal nucleotide of the primer and its template. The 3′ terminal nucleotide of the primer being either complementary or non-complementary to the specific mutation, allele or polymorphism to be detected. There is a selective advantage for primer extension from the primer whose 3′ terminal nucleotide complements the base mutation, allele or polymorphism. Those primers which have a 3′ terminal mismatch with the template sequence severely inhibit or prevent enzymatic primer extension. Polymerase chain reaction or unidirectional primer extension reactions therefore result in product amplification when the 3′ terminal nucleotide of the primer complements that of the template, but not, or at least not efficiently, when the 3′ terminal nucleotide does not complement that of the template.

It will be appreciated that the test sample may equally be a nucleic acid sequence corresponding to the sequence in the test sample, that is to say that all or a part of the region in the sample nucleic acid may firstly be amplified using any convenient technique e.g. polymerase chain reaction (PCR), before analysis. The nucleic acid may be genomic DNA or fractionated or whole cell RNA. In one embodiment the RNA is whole cell RNA and is used directly as the template for labelling a first strand cDNA using random primers or poly A primers. The nucleic acid or protein in the test sample may be extracted from the sample according to standard methodologies (Sambrook et al. “Molecular Cloning—A Laboratory manual”, second edition. Cold Spring Harbor, N.Y. (1989)).

It will be apparent that the gene sequence disclosed for GT (as depicted in SEQ ID NO: 1) is a representative sequence. In normal individuals there are two copies of each gene, a maternal and paternal copy, which will likely have some sequence differences, moreover within a population there will exist numerous allelic variants of the gene sequence (natural allelic variants). It will be appreciated that the diagnostic methods and other aspects of this invention extend to the detection etc. of any of these sequence variants. Preferred sequence variants are those that possess at least 90% and preferably at least 95% sequence identity (nucleic acid or amino acid) to GT depicted in SEQ ID No. 1 or 2. Nucleic acid sequence identity can also be gauged by hybridisation studies whereby, under stringent hybridisation and wash conditions, only closely related sequences (for example, those with >90% identity) are capable of forming a hybridisation complex.

The amino acid sequence method for diagnosis is preferably one which is determined by immunological methods such as enzyme linked immunosorbent assay (ELISA).

The levels of the GT can be assessed from relative amounts of mRNA, cDNA, genomic DNA or polypeptide sequence present in the test sample. Where RNA is used, it may be desired to convert the RNA to a complementary cDNA and during this process it may be desirable to incorporate a suitable detectable label into the cDNA.

In a particular embodiment the method of the invention relies on detection of mRNA transcript levels. This involves assessment of the relative mRNA transcript levels of GT in a sample, and comparison of sample data to control data. The gene transcript can be detected individually, or, is preferably detected amongst a panel of other disease-linked gene GT from which a transcript profile can be generated. Levels of GT mRNA in the test sample can be detected by any technique known in the art. These include Northern blot analysis, reverse transcriptase-PCR amplification (RT-PCR), microarray analysis and RNAse protection.

In one embodiment, levels of GT RNA in a sample can be measured in a Northern blot assay. Here, tissue RNA is fractionated by electrophoresis, fixed to a solid membrane support, such as nitrocellulose or nylon, and hybridised to a probe or probes capable of selectively hybridising with the GT RNA to be detected. The actual levels may be quantitated by reference to one or more control housekeeping genes. Probes may be used singly or in combination. This may also provide information on the size of mRNA detected by the probe. Housekeeping genes are genes which are involved in the general metabolism or maintenance of the cell, and are considered to be expressed at a constant level irrespective of cell type, physiological state or stage in the cell cycle. Examples of suitable housekeeping genes are: beta actin, GAPDH, histone H3.3 or ribosomal protein L13 (Koehler et al., Quantitation of mRNA by Polymerase Chain Reaction. Springer-Verlag, Germany (1995)).

To gauge relative expression levels, a control sample can be run alongside the test sample or, the test result/value can be compared to GT expression levels expected in a normal or control tissue. These control values can be generated from prior test experiments using normal or control tissues, to generate mean or normal range values for GT.

In another embodiment, the GT nucleic acid in a tissue sample is amplified and quantitatively assayed. The polymerase chain reaction (PCR) procedure can be used to amplify specific nucleic acid sequences through a series of iterative steps including denaturation, annealing of oligonucleotide primers (designed according to the sequence disclosed in SEQ ID NO. 1), and extension of the primers with DNA polymerase (see, for example, Mullis, et al., U.S. Pat. No. 4,683,202; Loh et al., Science 243:217, 1988). In reverse transcriptase-PCR (RT-PCR) this procedure is preceded by a reverse transcription step to allow a large amplification of the number of copies of mRNA (Koehler et al., supra). Other known nucleic acid amplification procedures include transcription-based amplification systems (TAS) such as nucleic acid based sequence application (NASBA) and 3SR (Kwoh et al., Proc Natl. Acad Sci USA 86:1173, 1989), Gingeras et al., PCT application WO 88/10315), the ligase chain reaction (LCR, see European Application No. 320308), Strand Displacement Amplification (SDA), “race”, “one sided PCR” and others (Frohman, PCR Protocols: a Guide to Methods and Applications. Academic Press, NY (1990); Ohara et al., Proc Natl Acad. Sci. USA 86:5673-5677, 1989)). Quantitation of RT-PCR products can be done while the reaction products are building up exponentially, and can generate diagnostically useful clinical data. In one embodiment, analysis is carried out by reference to one or more housekeeping genes, which are also amplified by RT-PCR. Quantitation of RT-PCR product may be undertaken, for example, by gel electrophoresis visual inspection or image analysis, HPLC (Koehler et al., supra) or by use of fluorescent detection methods such as intercalation labelling, Taqman probe (Higuchi et al., Biotechnology 10:413-417, 1992), Molecular Beacon (Piatek et al., Nature Biotechnol. 4:359-363, 1998), primer or Scorpion primer (Whitcombe et al., Nature Biotech 17:804-807, 1999); or other fluorescence detection method, relative to a control housekeeping gene or genes as discussed above.

GT RNA measurements can also be carried out on sinovial fluid, blood or serum samples. Preferably, the RNA is obtained from a peripheral blood sample. In the case of soluble RNA in the blood serum, the low abundance of mRNA expected would necessitate a sensitive test such as RT-PCR (Kopreski et al., Clin Cancer Res 5:1961-5, 1999). A whole blood gradient may be performed to isolate nucleated cells and total RNA is extracted such as by the Rnazole B method (Tel-Test Inc., Friendsworth, Tex.) or by modification of methods known in the art such as described in Sambrook et al., (supra).

In one embodiment of the invention, the diagnosis/detection method of the invention involves assessing GT transcript levels using microarray analysis. Microarray technology makes it possible to simultaneously study the expression of many thousands of genes in a single experiment. Analysis of gene expression in human tissue (e.g. biopsy or post mortem tissue) can assist in the diagnosis and prognosis of disease and the evaluation of risk for disease. A comparison of levels of expression of various genes from patients with defined pathological disease conditions with normal patients enables an expression profile, characteristic of disease, to be created.

Probes are made that selectively hybridise to the sequences of the target GT gene in the test sample. These probes, perhaps together with other probes and control probes, are bound at discrete locations on a suitable support medium such as a nylon filter or microscope slide to form a transcript profiling array. The diagnostic method involves assessing the relative mRNA transcript level of the GT in a clinical sample. This can be done by radioactively labelling, or non-radioactively labelling the tissue mRNA, which can be optionally purified from total RNA, in any of a number of ways well known to the art (Sambrook et al., supra). The probes can be directed to any part, or all, of the target GT mRNA.

In another embodiment of the invention, total GT RNA or DNA is quantified and compared to levels in control tissue or expected levels from pre tested standards. DNA and/or RNA may be quantified using techniques well known in the art. Messenger RNA is often quantitated by reference to internal control mRNA levels within the sample, often relative to housekeeping genes (Koehler et al., supra).

In a particular embodiment, hybridisation signals generated are measured by computer software analysis of images on phosphorimage screens exposed to radioactively labelled tissue RNA hybridised to a microarray of probes on a solid support such as a nylon membrane. In another, quantities are measured by densitometry measurements of radiation-sensitive film (e.g. X-ray film), or estimated by visual means. In another embodiment quantities are measured by use of fluorescently labelled probe, which may be a mixture of biopsy and normal RNA differentially labelled with different fluorophores, allowing quantities of GT mRNA to be expressed as a ratio versus the normal level. The solid support in this type of experiment is generally a glass microscope slide, and detection is by fluorescence microscopy and computer imaging.

The detection of specific interactions may be performed by detecting the positions where the labelled target sequences are attached to the array. Radiolabelled probes can be detected using conventional autoradiography techniques. Use of scanning autoradiography with a digitised scanner and suitable software for analysing the results is preferred. Where the label is a fluorescent label, the apparatus described, e.g. in International Publication No. WO 90/15070; U.S. Pat. No. 5,143,854 or U.S. Pat. No. 5,744,305 may be advantageously applied. Indeed, most array formats use fluorescent readouts to detect labelled capture:target duplex formation. Laser confocal fluorescence microscopy is another technique routinely in use (Kozal et al., Nature Medicine 2:753-759, 1996). Mass spectrometry may also be used to detect oligonucleotides bound to a DNA array (Little et al, Analytical Chemistry 69: 4540-4546, 1997). Whatever the reporter system used, sophisticated gadgetry and software may be required in order to interpret large numbers of readouts into meaningful data (such as described, for example, in U.S. Pat. No. 5,800,992 or International Publication No. WO 90/04652).

In a particular embodiment of the microarray test, the GT RNA measurement is generated as a value relative to an internal standard (i.e. a housekeeping gene) known to be constant or relatively constant. The histone H3.3 and ribosomal protein L19 housekeeping genes have been shown to be cell-cycle independent and constitutively expressed in all tissues (Koehler et al., supra). For normalisation of data, several different housekeeping genes can be used to generate an average housekeeping measurement.

A microarray or RT-PCR test to detect OA disorder or susceptibility thereto can be used where tissue samples containing mRNA are available.

Samples for RNA extraction must be treated promptly to avoid RNA degradation (Sambrook et al., supra). This entails either prompt extraction using e.g. phenol-based reagents or snap freezing in e.g. liquid Nitrogen. Samples can be stored at −70° C. or less until RNA can be extracted at a later date. Proprietary reagents are available which allow tissue or cells to be conveniently stored for several days at room temperature and up to several months at 4° C. (e.g. RNA later, Ambion Inc., TX). Prior to extraction, methods such as grinding, blending or homogenisation are used to dissipate the tissue in a suitable extraction buffer. Typical protocols then use solvent extraction and selective precipitation techniques.

In another embodiment oligonucleotide probe(s) capable of selectively hybridising to GT nucleic acid, can be used to detect levels of GT gene expression.

Convenient DNA sequences for use in the various aspects of the invention may be obtained using conventional molecular biology procedures, for example by probing a human genomic or cDNA library with one or more labelled oligonucleotide probes containing 10 or more contiguous nucleotides designed using the nucleotide sequences described here. Alternatively, pairs of oligonucleotides one of which is homologous to the sense strand and one to the antisense strand, designed using the nucleotide sequences described here to flank a specific region of DNA may be used to amplify that DNA from a cDNA library.

Primers or probes for use in any of the methods of the invention may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7, 1993; 1^{st} Edition. If required the primer(s) may be labelled to facilitate detection.

The oligonucleotide primers and probes of the invention are particularly suitable for detecting the genotype of a particular SNP of GT.

There are many conventional detectable labels such as radioisotopes, fluorescent labels, chemiluminescent compounds, labelled binding proteins, magnetic labels, spectroscopic markers and linked enzymes that might be used in conjunction with the primers or probes of the invention. One particular example well known in the art is end-labelling with ³²P. Fluorescent labels are preferred because they are less hazardous than radiolabels, they provide a strong signal with low background and various different fluorophors capable of absorbing light at different wavelengths and/or giving off different colour signals exists to enable comparative analysis in the same analysis. For example, fluorescein gives off a green colour, rhodamine gives off a red colour and both together give off a yellow colour.

Preferred primers for amplification are between 15 and 60 bp, more preferably between 17 and 35 bp in length. Probe sequences can be anything from about 25 nucleotides in length upwards. If the target sequence is a gene of 2 kb in size the probe sequence can be the complete gene sequence complement and thus may also be 2 kb in size.

Preferably, the probe sequence is a genomic, or more preferably a cDNA, fragment of the target sequence and may be between 50 and 2000 bp, preferably between 200 and 750 bp. It will be appreciated that multiple probes each capable of selectively hybridising to a different target sequence of the GT nucleic acid, maybe across the complete length of the GT gene sequence, may be prepared and used together in a diagnostic test. The primers or probes may be completely homologous to the target sequence or may contain one or more mismatches to assist specificity in binding to the correct template sequence. Any sequence which is capable of selectively hybridising to the target sequence of interest may be used as a suitable primer or probe sequence. It will also be appreciated that the probe or primer sequences must hybridise to the target template nucleic acid. If the target nucleic acid is double stranded (genomic or cDNA) then the probe or primer sequence can hybridise to the sense or antisense strand. If however the target is mRNA (single stranded sense strand) the primer/probe sequence will have to be the antisense complement.

An example of a suitable hybridisation solution when a nucleic acid is immobilised on a nylon membrane and the probe nucleic acid is greater than 500 bases or base pairs is: 6×SSC (saline sodium citrate), 0.5% SDS (sodium dodecyl sulphate), 100 μg/ml denatured, sonicated salmon sperm DNA. The hybridisation being performed at 68° C. for at least 1 hour and the filters then washed at 68° C. in 1×SSC, or for higher stringency, 0.1×SSC/0.1% SDS.

An alternate example of “stringent hybridisation conditions” provides for an overnight incubation at 42° C. in a solution comprising 50% formamide, 5×SSC (750 mM NaCl, 75 mM trisodium citrate), 50 mM sodium phosphate (pH 7.6), 5×Denhardt's solution, 10% dextran sulphate, and 20 pg/ml denatured, sheared salmon sperm DNA, followed by washing the filters in 0.1×SSC at 65° C. for at least an hour.

An example of a suitable hybridisation solution when a nucleic acid is immobilised on a nylon membrane and the probe is an oligonucleotide of between 12 and 50 bases is: 3M trimethylammonium chloride (TMACl), 0.01M sodium phosphate (pH 6.8), 1 mM EDTA (pH 7.6), 0.5% SDS, 100 μg/ml denatured, sonicated salmon sperm DNA and 0.1 dried skimmed milk. The optimal hybridisation temperature (Tm) is usually chosen to be 5° C. below the Ti of the hybrid chain. Ti is the irreversible melting temperature of the hybrid formed between the probe and its target. If there are any mismatches between the probe and the target, the Tm will be lower. As a general guide, the recommended hybridisation temperature for 17-mers in 3M TMACl is 48-50° C.; for 19-mers, it is 55-57° C.; and for 20-mers, it is 58-66° C.

Levels of GT gene expression can also be detected by screening for levels of polypeptide (GT protein). For example, monoclonal antibodies immunoreactive with GT protein can be used to screen a test sample. Such immunological assays can be done in any convenient format known in the art. These include Western blots, immunohistochemical assays and ELISA assays. Functional assays can also be used, such as protein binding determinations.

The GT protein of the invention and homologues or fragments thereof may be used to generate substances which selectively bind to it and in so doing regulate the activity of the protein. Such substances include, for example, antibodies, and the invention extends in particular to an antibody which is capable of binding to the protein shown in SEQ ID No:2. In particular the antibody may be a neutralizing antibody.

As used herein the term antibody is to be understood to mean a whole antibody or a fragment thereof, for example a F(ab)2, Fab, FV, VH or VK fragment, a single chain antibody, a multimeric monospecific antibody or fragment thereof, or a bi- or multi-specific antibody or fragment thereof. Each of these types of antibody derivative and their acronyms are well known to the person skilled in the art.

In another particular embodiment, antibodies directed against GT protein can be used, to detect, prognose, diagnose and stage OA disease. Various histological staining methods known in the art, including immunochemical staining methods, may also be used. Silver stain is but one method of detecting GT proteins. For other staining methods useful in the present invention see, for example, A Textbook of Histology, Eds. Bloom and Fawcett, W.B. Saunders Co., Philadelphia (1964).

According to a further aspect of the invention there is provided use of an antibody selective for GT protein, in an assay to diagnose or prognose or monitor OA. The antibodies for use in this aspect of the invention can be prepared using the GT protein/polypeptides.

Methods of making and detecting labelled antibodies are well known (Campbell; Monoclonal Antibody Technology, in: Laboratory Techniques in Biochemistry and Molecular Biology, Volume 13. Eds: Burdon R et al. Elsevier, Amsterdam (1984)). The term antibody includes both monoclonal antibodies, which are a substantially homogeneous population, and polyclonal antibodies, which are heterogeneous populations. The term also includes inter alia, humanized and chimeric antibodies. Monoclonal antibodies to specific antigens may be obtained by methods known to those skilled in the art, such as from hybridoma cells, phage display libraries or other methods. Monoclonal antibodies may be inter alia, human, rat or mouse derived. For the production of human monoclonal antibodies, hybridoma cells may be prepared by fusing spleen cells from an immunised animal, e.g. a mouse, with a tumour cell. Appropriately secreting hybridoma cells may thereafter be selected (Koehler & Milstein, Nature 256:495-497, 1975); Cole et al., “Monoclonal antibodies and Cancer Therapy”, Alan R Liss Inc, New York N.Y. pp 77-96, 1985). Such antibodies may be of any immunoglobulin class including IgG, IgM, IgE, IgA, IgD and any subclass thereof.

Polyclonal antibodies can be generated by immunisation of an animal (such as a mouse, rat, goat, horse, sheep etc) with an antigen, such as one of the GT protein used in this invention.

The GT polypeptide(s) can be prepared by various techniques known to the person skilled in the art. RNA transcripts can be used to prepare a polypeptide of the invention by in vitro translation techniques according to known methods (Sambrook et al. supra). Alternatively, the GT polypeptide(s) can be synthesised chemically. For example, by the Merryfield technique (J. Amer. Chem. Soc. 85:2149-2154, 1968). Numerous automated polypeptide synthesizers, such as Applied Biosystems 431A Peptide Synthesizer also now exist. Alternatively, the GT polypeptide(s) are produced from a nucleotide sequence encoding the polypeptide using recombinant expression technology. A variety of expression vector/host systems may be used to express the GT coding sequences. These include, but are not limited to microorganisms such as bacteria expressed with plasmids, cosmids or bacteriophage; yeasts transformed with expression vectors; insect cell systems transfected with baculovirus expression systems; plant cell systems transfected with plant virus expression systems, such as cauliflower mosaic virus; or mammalian cell systems (for example those transfected with adenoviral vectors); selection of the most appropriate system is a matter of choice. Preferably, the GT protein is expressed in eukaryotic cells, especially mammalian, insect and yeast cells. Mammalian cells provide post-translational modifications to recombinant GT protein, which include folding and/or phosphorylation. Expression vectors usually include an origin of replication, a promoter, a translation initiation site, optionally a signal peptide, a polyadenylation site, and a transcription termination site. These vectors also usually contain one or more antibiotic resistance marker gene(s) for selection. As noted above, suitable expression vectors may be plasmids, cosmids or viruses such as phage or retroviruses. The coding sequence of the polypeptide is placed under the control of an appropriate promoter, control elements and transcription terminator so that the nucleic acid sequence encoding the polypeptide is transcribed into RNA in the host cell transformed or transfected by the expression vector construct. The coding sequence may or may not contain a signal peptide or leader sequence for secretion of the polypeptide out of the host cell. Expression and purification of the GT polypeptide(s) can be easily performed using methods well known in the art (for example as described in Sambrook et al. supra).

The GT polypeptide(s) so produced can also be used to inoculate animals, from which serum samples, containing the specific antibody against the introduced GT protein/polypeptide, can later be obtained.

Rodent antibodies may be humanized using recombinant DNA technology according to techniques known in the art. Alternatively, chimeric antibodies, single chain antibodies, Fab fragments may also be developed against the polypeptides of the invention (Huse et al., Science 256:1275-1281, 1989), using skills known in the art. Antibodies so produced have a number of uses, which will be evident to the molecular biologist or immunologist skilled in the art. Such uses include, but are not limited to, monitoring enzyme expression, development of assays to measure enzyme activity and use as a therapeutic agent. Enzyme linked immunosorbant assays (ELISAs) are well known in the art and would be particularly suitable for detecting the GT protein or polypeptide fragments thereof in a test sample.

The GT specific antibodies can be used in an ELISA assay to detect GT protein in body fluids or by immunohistochemistry or other means. In addition, an antibody could be used individually or as part of a panel of antibodies, together with a control antibody, which reacts to a common protein, on a dipstick or similar diagnostic device.

All the essential materials and reagents required for detecting GT in a test sample may be assembled together in a kit. Such a kit may comprise one or more diagnostic cDNA probes or oligonucleotide primers together with control probes/primers. The kit may contain probes immobilised on a microarray substrate such as a filter membrane or silicon-based substrate. The kit may also comprise samples of total RNA derived from tissues of various physiological states, such as normal, BPH, confined tumour and metastatic tumour, for example, to be used as controls. The kit may also comprise appropriate packaging and instructions for use in the methods of the invention.

According to another aspect of the present invention there is provided a diagnostic kit for diagnosing or prognosing or monitoring OA comprising, one or more diagnostic probe(s) and/or diagnostic primer(s) and/or antibodies capable of selectively hybridising or binding to GT.

It will be appreciated that the term “diagnostic kit” is not intended to limit the kit to diagnostic use only, it also encompasses other uses such as in prognostic, stage monitoring and therapeutic efficacy studies.

In a preferred embodiment, the diagnostic (detection) probes are provided on a microarray.

Such kits may further comprise appropriate buffer(s) and/or polymerase(s) such as thermostable polymerases, for example taq polymerase. They may also comprise companion/constant primers and/or control primers or probes. A companion/constant primer is one that is part of the pair of primers used to perform PCR. Such primer usually complements the template strand precisely. The kit may also contain control normal osteoarthritis RNA labelled with one fluorophore (E.g. Cy5). In use, patient RNA derived from biopsy or body fluids or cells can be labelled with another fluorophore (e.g. Cy3), the RNAs could then be mixed and hybridised to the array. Instrumentation to detect fluorescence ratio e.g. of. Cy3:Cy5 are available and could be used to detect GT over-expression.

In another embodiment the kit comprises one or more specific probes suitable for hybridisation to mRNA in tissue sections in situ. The kit may also contain hybridisation buffer and detection reagents for colorimetric or fluorescence microscopy detection. In another embodiment the kit comprises a set of specific oligonucleotide primers, optionally labelled, for quantitation by RT-PCR of GT mRNA. These primers may be Scorpion primers (Whitcombe et al., Nature Biotechnol. 17:804-807, 1999) allowing accurate quantitation of specific PCR product. Alternatively, Taqman or Molecular Beacon probes may be provided in the kit for this purpose. One form of the kit would be a microtitre plate containing specific reagents in several wells, to which aliquots of extracted RNA could be pipetted. The microtitre plate could be thermocycled on a suitable machine, which could also be capable of reading fluorescence emissions from plate wells (e.g. Perkin Elmer 7700).

In another embodiment the kit comprises one or more antibodies specific for the GT protein for use in immunohistochemical analysis.

In another embodiment the kit is an ELISA kit comprising one or more antibodies specific for the GT protein identified herein.

In another aspect of the invention there is provided a method for treating a patient suffering from OA comprising administering to said patient an effective amount of an antibody specific for GT.

According to another aspect of the invention, the GT gene may be used in gene therapy, for example where it is desired to modify the production of the protein in vivo, and the invention extends to such uses.

Pathway mapping may be used to determine each protein in the cell with which the GT protein interacts and, in turn, the proteins with which each of these proteins interacts also. In this way it is possible to identify the specific critical signaling pathway which links the disease stimulus to the cell's response thereby enabling the identification of new potential targets for therapy intervention.

According to a further aspect of the invention there is provided the use of the GT gene or a fragment thereof in research to identify further gene targets implicated in OA.

Knowledge of the gene according to the invention also provides the ability to regulate its expression in vivo by for example the use of antisense DNA or RNA. One therapeutic means of inhibiting or dampening the expression levels of a particular gene (for example GT identified herein) is to use antisense therapy. Antisense therapy utilises antisense nucleic acid molecules that are synthetic segments of DNA or RNA (“oligonucleotides”), designed to mirror specific mRNA sequences and block protein production. Once formed, the mRNA binds to a ribosome, the cell's protein production “factory” which effectively reads the RNA sequence and manufactures the specific protein molecule dictated by the gene. If an antisense molecule is delivered to the cell (for example as native oligonucleotide or via a suitable antisense expression vector), it binds to the messenger RNA because its sequence is designed to be a complement of the target sequence of bases. Once the two strands bind, the mRNA can no longer dictate the manufacture of the encoded protein by the ribosome and is rapidly broken down by the cell's enzymes, thereby freeing the antisense oligonucleotide to seek and disable another identical messenger strand of mRNA.

Thus, according to another aspect of the invention there is provided a method for treating a patient suffering from OA comprising administering to said patient an effective amount of an anti-sense molecule capable of binding to the mRNA of the GT gene, and inhibiting expression of the protein product of the GT gene.

Complete inhibition of protein production is not essential, indeed may be detrimental. It is likely that inhibition to a state similar to that in normal tissues would be desired.

This aspect of antisense therapy is particularly applicable if the OA disorder is a direct cause of over-expression of the GT gene in question, although it is equally applicable if said GT gene indirectly cause the OA disorder. With knowledge of the GT gene and mRNA sequence, the person skilled in the art is able to design suitable antisense nucleic acid therapeutic molecules and administer them as required.

Antisense oligonucleotide molecules with therapeutic potential can be determined experimentally using well-established techniques. To enable methods of down-regulating expression of the GT gene of the present invention in mammalian cells, an example antisense expression construct can be readily constructed for instance using the pREP10 vector (Invitrogen Corporation). Transcripts are expected to inhibit translation of the gene in cells transfected with this type of construct. Antisense transcripts are effective for inhibiting translation of the native gene transcript, and capable of inducing the effects (e.g., regulation of tissue physiology) herein described. Oligonucleotides which are complementary to and hybridizable with any portion of GT gene mRNA are contemplated for therapeutic use. U.S. Pat. No. 5,639,595, “Identification of Novel Drugs and Reagents”, issued Jun. 17, 1997, wherein methods of identifying oligonucleotide sequences that display in vivo activity are thoroughly described, is herein incorporated by reference. Expression vectors containing random oligonucleotide sequences derived from the GT gene sequence are transformed into cells. The cells are then assayed for a phenotype resulting from the desired activity of the oligonucleotide. Once cells with the desired phenotype have been identified, the sequence of the oligonucleotide having the desired activity can be identified. Identification may be accomplished by recovering the vector or by polymerase chain reaction (PCR) amplification and sequencing the region containing the inserted nucleic acid material. Antisense molecules can be synthesised for antisense therapy. These antisense molecules may be DNA, stable derivatives of DNA such as phosphorothioates or methylphosphonates, RNA, stable derivatives of RNA such as 2′-O-alkylRNA, or other oligonucleotide mimetics. U.S. Pat. No. 5,652,355, “Hybrid Oligonucleotide Phosphorothioates”, issued Jul. 29, 1997, and U.S. Pat. No. 5,652,356, “Inverted Chimeric and Hybrid Oligonucleotides”, issued Jul. 29, 1997, which describe the synthesis and effect of physiologically-stable antisense molecules, are incorporated by reference. Antisense molecules may be introduced into cells by microinjection, liposome encapsulation or by expression from vectors harboring the antisense sequence.

As noted above, antisense nucleic acid molecules may also be provided as RNAs, as some stable forms of RNA are now known in the art with a long half-life that may be administered directly, without the use of a vector. In addition, DNA constructs may be delivered to cells by liposomes, receptor mediated transfection and other methods known to the art.

The antisense DNA or RNA for co-operation with the gene in SEQ ID No:1 can be produced using conventional means, by standard molecular biology and/or by chemical synthesis as described above. If desired, the antisense DNA or antisense RNA may be chemically modified so as to prevent degradation in vivo or to facilitate passage through a cell membrane and/or a substance capable of inactivating mRNA, for example ribozyme, may be linked thereto and the invention extends to such constructs. The antisense DNA or antisense RNA may be of use in the treatment of diseases or disorders in humans in which the over- or under-regulated production of the GT gene product has been implicated.

Alternatively, ribozyme molecules may be designed to cleave and destroy the GT mRNA in vivo. Ribozymes are RNA molecules that possess highly specific endoribonucleases activity. Hammerhead ribozymes comprise a hybridising region, which is complementary in nucleotide sequence to at least part of the target RNA, and a catalytic region, which is adapted to recognise and cleave the target RNA. The hybridising region preferably contains at least 9 nucleotides. The design, construction and use of such ribozymes is well known in the art and is more fully described in Haselhoff and Gerlach, (Nature. 334:585-591, 1988). In another alternative oligonucleotides designed to hybridise to the 5′-region of the GT gene so as to form triple helix structures may be used to block or reduce transcription of the GT gene. In another alternative, RNA interference (RNAi) oligonucleotides or short (18-25 bp) RNAi GT sequences cloned into plasmid vectors are designed to introduce double stranded RNA into mammalian cells to inhibit and/or result in the degradation of GT messenger RNA. GT RNAi molecules may begin adenine/adenine (AA) or at least (any base-A, U, C or G)A . . . and may comprise of 18 or 19 or 20 or 21 or 22 or 23, or 24 or 25 base pair double stranded RNA molecules with the preferred length being 21 base pairs and be specific to individual GT sequences with 2 nucleotide 3′ overhangs or hairpin forming 45-50mer RNA molecules. The design, construction and use of such small inhibitory RNA molecules is well known in the art and is more fully described in the following: Elbashir et al., (Nature. 411(6836):494-498, 2001); Elbashir et al., (Genes & Dev. 15:188-200, 2001); Harborth, J. et al. (J. Cell Science 114:4557-4565, 2001); Masters et al. (Proc. Natl. Acad. Sci. USA 98:8012-8017, 2001); and, Tuschl et al., (Genes & Dev. 13:3191-3197, 1999).

According to a further aspect of the invention there is provided a method of treating a human in need of treatment with a small molecule drug acting on the GT protein or an inhibitory nucleic acid molecule acting against the GT mRNA, which method comprises:

i) detection of a polymorphism in or flanking the GT gene in the human, which diagnosis preferably comprises determining the sequence at one or more positions selected from the group consisting of: positions: 5283, 21037, 21134, 25515, 34419, 59661, 59968 and 73821 . . . 73842 of the GT gene (as defined by the position in SEQ ID NO: 1), or a polymorphism in linkage disequilibrium above D' 0.9 therewith;
ii) determining the status of the human by reference to polymorphism in or flanking the GT gene; and,
iii) administering an effective amount of the drug that acts on the GT protein or an inhibitory nucleic acid molecule acting against the GT mRNA.

In another embodiment, this aspect is applied to the detection or one or more of the polymorphisms identified in Table 2.

According to a further aspect of the invention there is provided a method of treating a human in need of treatment with a small molecule drug acting on the GT protein or an inhibitory nucleic acid molecule acting against the GT mRNA, in which the method comprises:

i) measuring the level of the GT mRNA in a tissue sample obtained from the human and,
ii) determining the status of the human by reference to normal levels of the GT mRNA; and,
iii) administering an effective amount of the drug.

According to a further aspect of the invention there is provided a method of treating a human in need of treatment with a small molecule drug acting on the GT protein or an inhibitory nucleic acid molecule acting against the GT mRNA, in which the method comprises:

i) measuring the level of the GT protein in a tissue sample obtained from the human and,
ii) determining the status of the human by reference to normal levels of the GT protein; and,
iii) administering an effective amount of the drug.

According to a further aspect of the invention there is provided a method of treatment of a patient suffering from an OA disorder, comprising administration to the patient of a compound capable of reducing the transcription or expression of GT.

According to a further aspect of the invention there is provided a method of treatment of a patient suffering from an OA disorder, comprising administration to the patient an inhibitory nucleic acid molecule targeted against the mRNA of GT.

According to a further aspect of the invention there is provided the u se of an inhibitory nucleic acid molecule or an antibody directed against GT, in the manufacture of a medicament for treating an OA disorder.

In each of the above aspects of the invention, the “inhibitory nucleic acid molecule” is selected from the group consisting of: an antisense, ribozyme, triple helix aptemer and RNAi molecule.

Each aspect of the invention is not only applicable to OA, including primary and secondary OA but others arthropathies, such as intervertebral disc degeneration and temporomandibular joint disease; metabolic arthropies such as chronic crystal deposition disease including CPPD and hydroxyapatite as well as contributing to inflammatory disease such as rheumatoid arthritis.

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods. See, generally, Sambrook and Russell, Molecular Cloning: A Laboratory Manual, 3d ed. (2001) Cold Spring Harbor Laboratory Press, Cold Spring Harbor, N.Y. and Ausubel et al., Short Protocols in Molecular Biology (1999) 4^{th} Ed, John Wiley & Sons, Inc.; as well as Guthrie et al., Guide to Yeast Genetics and Molecular Biology, Methods in Enzymology, Vol. 194, Academic Press, Inc., (1991), PCR Protocols: A Guide to Methods and Applications (Innis, et al. 1990. Academic Press, San Diego, Calif.), McPherson et al., PCR Volume 1, Oxford University Press, (1991), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). These documents are incorporated herein by reference.

The invention will be further described by way of the following non-limiting examples:

### EXAMPLE 1

### Identification of GT as a Gene Associated with OA

We investigated the 11 q region of interest using a linkage-based approach based on SNP genotyping.

### 1. Methods

The 20 cM 11 q region described in WO 00/20632, is delimited by the microsatellite markers D11S4046 and D11S1320. Linkage disequilibrium analysis identified excess transmission of the 258 bp allele of marker D11S937 in cases (i.e. patients diagnosed with OA who had undergone joint replacement) compared to controls (e.g. asymptomatic spouses of patients).

In order to finely map of this region we performed SNP discovery and genotyping to decrease the inter-marker distance. Genotyping was carried out on 600 DNAs from subjects suffering from OA of the knee or hip and who had undergone joint replacement. Genotyping of 500 control DNAs from the asymptomatic spouses of the cases was also performed.

### A. SNP Discovery

SNPs occurring between the markers D11S4046 and D11S1320 were identified by the following methods.

(i) BAC (Bacterial Artificial Chromosome) shotgun library screening

Hybridisation against the dejong RCPI-11 BAC library (Osoegawa et al. Genome Research. 11 (3):483-496, 2001) was undertaken with pools of ³²P end labelled oligos derived from STSs (Sequence Tagged Sites) in the region of interest. BACs were then checked individually by PCR and 137 individual BACs were isolated.

BAC DNA was prepared from 500 ml overnight cultures using an extraction kit (Nucleon/Tepnel, Manchester UK). Two methods were used to construct libraries. In the first method, BAC DNA was partially digested with Sau3a and separated by agarose gel electrophoresis. Fragments in the 0.5-4 kb size range were cut out of the gel and purified. The fragments were shotgun cloned into the pZERO plasmid vector (Invitrogen) and single colonies were picked and the inserts were PCR amplified using conventional T7 and SP6 primers. PCR products were sequenced using dye terminator chemistry. In the second method, BAC DNA was sheared to 1 Kb in size using a Hydroshear (GeneMachines, San Carlos, Calif.) and sub-cloned into the pZERO plasmid vector. Single colonies were picked and the inserts were PCR amplified using conventional M13 forward and reverse primers. PCR products were sequenced on the MegaBace (Amersham Pharmacia, Little Chalfont, UK) using M13 F&R primers.

Sequences generated from the PCR products in all libraries were assembled into a contig using the SeqMan package (DNAStar, Madison Wis.). Sequences over 500 bp in size were analysed, trimmed for vector and quality and masked for repeats/E. coli DNA and novel BAC sequence was identified. Primers were designed to this sequence to generate 400-600 bp PCR products for use in the polymorphism screening protocols. A total of 837 STSs were generated for screening. All primer pairs were tested in 3 separate amplification conditions and working primers were put through a polymorphism screen in 1 pool (17 individuals) and 3 individuals were sequenced in the forward and reverse direction. Analysis of the sequences was done using Phred/Phrap/Consed.

Analysis of the shotgun libraries generated a total of 132 SNPs. An average of 1 SNP was discovered for every 1.6 kb sequence analysed.

### (ii) Analysis of Genomic Clones

We identified 330 genomic clones (Research Genetics) mapping to the region of interest and were able to use the sequences directly to provide sequence for polymorphism screening. The Genomic clone sequence was used to design at least 6 primer pairs for each clone and subsequent screening was performed by a combination of WAVE (Transgenomic Inc. Crewe, UK) and sequence analysis.

141 SNPs were discovered by this method.

(iii) The SNP Consortium SNPs

The fourth release of The SNP Consortium's database of human single nucleotide polymorphisms contained 102,719 SNPs, all of which were anchored to the human genome by radiation hybrid mapping and/or “in silico” mapping to the genomic sequence working draft.

A total of 168 uniquely represented SNPs were retrieved from the 20 Mb region of interest.

### B. SNP Genotyping

The SNPs discovered in Step A were genotyped in 600 DNAs from subjects suffering from OA of the knee or hip and who had undergone joint replacement and 500 control DNAs from the asymptomatic spouses of the cases. The genotyping was carried out using allele specific amplification (ARMS) and single nucleotide primer extension.

### C. Microsatelite Genotyping

Ten microsatellite markers, D11S916, D11S1321, D11S4081, D11S4128, D11S4179, D11S911, D11S4079, D11S906, D11S937, D11S4166, were genotyped in the case control cohorts.

### EXAMPLE 2

### Linkage Analysis

### A. Microsatellites

Analysis of the microsatellites was carried out in the program CLUMP (author D. Curtis, Human Genome Mapping Project). This program compares the distribution of alleles across cases and controls generating a single p value, thus negating the need for correction of multiple tests for each allele (not each marker). The program also carries out monte-carlo simulations, producing an exact p value for the data.

The association was confirmed for D11S937 (p=0.04) and a further association was seen with D11S4081 (p 0.02), which maps approximately 0.7 cM from D11S937.

### B. SNPs

Allele frequencies were compared for each SNP between cases and controls, ODDs ratios were calculated with 95% confidence intervals (Jurg Ott, Analysis of Human Genetic Linkage 3^{rd} Ed, John Hopkins Univ. Press, 1999). Analysis of SNPs with an allele frequency of >5% which were in Hardy-Weinberg equilibrium revealed 9 (SEQ ID Nos: 4-11 & Table 3) which showed significant association with the disease (p.01-0.05). These were not corrected for multiple testing. It was found that all 9 SNPs lay within the same 3 cM radiation hybrid interval (termed interval D), which also mapped close to microsatelite marker D11S937 located at 11q13.5.

### EXAMPLE 3

### Gene Discovery

35 Bacterial Artificial Chromosomes (BACs) were found to map to interval D. Following analysis using Seqman. The BACs were assembled into 138 contigs covering 2.8 Mb. The gene termed GT was found within this interval.

### EXAMPLE 4

### Polymorphism Discovery

Following publication of the Ensemble sequence for this region the cDNA for GT was mapped to the genomic sequence to identify the exon/intron boundaries, the UTRs and potential promotor areas.

PCR primers were designed to span the exons, UTRs, potential promotor areas and the flanking intronic sequence. Sequencing was carried out in 29 unrelated Caucasian individuals and the sequence was aligned in Polyphred/Phrap/Consed to identify polymorphisms.

The polymorphisms discovered are shown in Table 2.

TABLE 2
Features of GTOA gene and flanking genomic DNA
LOCATION is by nucleotide number and refers to SEQ ID NO. 1

| LOCATION | FEATURE TYPE | FEATURE ID |
|---|---|---|
| 7 | SNP | OA_1 |
| 23 | SNP | OA_2 |
| 79 | SNP | OA_3 |
| 453 | SNP | OA_4 |
| 5211 | SNP | OA_5 |
| 5283 | SNP | OA_6 |
| 5615 | SNP | OA_7 |
| 5861 | SNP | OA_8 |
| 7137 | SNP | OA_9 |
| 15035 | SNP | OA_10 |
| 16307 | SNP | OA_11 |
| 19438 | SNP | OA_12 |
| 19876 | SNP | OA_13 |
| 20055 | SNP | OA_14 |
| 20141 | SNP | OA_15 |
| 20664 | SNP | OA_16 |
| 20870 | SNP | OA_17 |
| 21010 | SNP | OA_18 |
| 21037 | SNP | OA_19 |
| 21099 | SNP | OA_20 |
| 21134 | SNP | OA_21 |
| 21157 | SNP | OA_22 |
| 21161 | SNP | OA_23 |
| 21271 | SNP | OA_24 |
| 21467 | SNP | OA_25 |
| 22351 | SNP | OA_26 |
| 22993 | SNP | OA_27 |
| 24203 | SNP | OA_28 |
| 24340 | SNP | OA_29 |
| 24564 | SNP | OA_30 |
| 24785 | SNP | OA_31 |
| 25515 | SNP | OA_32 |
| 29621 | SNP | OA_33 |
| 29624 | SNP | OA_34 |
| 29814 | SNP | OA_35 |
| 29817 | SNP | OA_36 |
| 29892 | SNP | OA_37 |
| 32701 | SNP | OA_38 |
| 32752 | SNP | OA_39 |
| 32804 | SNP | OA_40 |
| 32876 | SNP | OA_41 |
| 34178 | SNP | OA_42 |
| 34206 | SNP | OA_43 |
| 34208 | SNP | OA_44 |
| 34210 | SNP | OA_45 |
| 34419 | SNP | OA_46 |
| 34464 | SNP | OA_47 |
| 34652 | SNP | OA_48 |
| 34704 | SNP | OA_49 |
| 37610 | SNP | OA_50 |
| 39874 | SNP | OA_51 |
| 42279 | SNP | OA_52 |
| 42513 | SNP | OA_53 |
| 42650 | SNP | OA_54 |
| 44138 | SNP | OA_55 |
| 44232 | SNP | OA_56 |
| 44405 | SNP | OA_57 |
| 44422 | SNP | OA_58 |
| 47280 | SNP | OA_59 |
| 47889 | SNP | OA_60 |
| 57999 | SNP | OA_61 |
| 58339 | SNP | OA_62 |
| 59333 | SNP | OA_63 |
| 59604 | SNP | OA_64 |
| 59661 | SNP | OA_65 |
| 59947 | SNP | OA_66 |
| 59952 | SNP | OA_67 |
| 59953 | SNP | OA_68 |
| 59960 | SNP | OA_69 |
| 59968 | SNP | OA_70 |
| 60324 | SNP | OA_71 |
| 60365 | SNP | OA_72 |
| 61251 | SNP | OA_73 |
| 63550 | SNP | OA_74 |
| 64398 | SNP | OA_75 |
| 64501 | SNP | OA_76 |
| 65068 | SNP | OA_77 |
| 65321 | SNP | OA_78 |
| 65649 | SNP | OA_79 |
| 66838 | SNP | OA_80 |
| 69177 | SNP | OA_81 |
| 69888 | SNP | OA_82 |
| 70542 | SNP | OA_83 |
| 70573 | SNP | OA_84 |
| 70712 | SNP | OA_85 |
| 71863 | SNP | OA_86 |
| 73167 | SNP | OA_87 |
| 73323 | SNP | OA_88 |
| 74102 | SNP | OA_89 |
| 74154 | SNP | OA_90 |
| 74724 | SNP | OA_91 |
| 75080 | SNP | OA_92 |
| 75105 | SNP | OA_93 |
| 80925 | SNP | OA_94 |
| 81960 | SNP | OA_95 |
| 81995 | SNP | OA_96 |
| 82020 | SNP | OA_97 |
| 82437 | SNP | OA_98 |
| 82906 | SNP | OA_99 |
| 21132 . . . 21385 | exon | EXON14 |
| 23064 . . . 23138 | exon | EXON_13 |
| 24170 . . . 24242 | exon | EXON_12 |
| 24546 . . . 24643 | exon | EXON_11 |
| 26997 . . . 27136 | exon | EXON_10 |
| 29632 . . . 29771 | exon | EXON_9 |
| 32840 . . . 32960 | exon | EXON_8 |
| 34076 . . . 34179 | exon | EXON_7 |
| 34452 . . . 34582 | exon | EXON_6 |
| 39372 . . . 39439 | exon | EXON_5 |
| 41255 . . . 41398 | exon | EXON_4 |
| 44211 . . . 44404 | exon | EXON_3 |
| 47548 . . . 47626 | exon | EXON_2 |
| 59663 . . . 59843 | exon | EXON_1 |
| 63501 . . . 63551 | microsatellite | D11S937 |
| 73822 . . . 73841 | INDEL | OA_100 |

TABLE 3
Polymorphisms showing positive association to OA
(ODDs ratio with p value <0.05)

| | | | Allele | |
|---|---|---|---|---|
| | SNP location | | showing | |
| | within SEQ ID | Nucleotide | association | |
| Feature ID | No. 1 | Change | with OA | Sequence ID |
| OA_6 | 5283 | C to A | C | SEQ ID No: 4 |
| OA_19 | 21037 | T to C | T | SEQ ID No: 5 |
| OA_21 | 21134 | C to T | T | SEQ ID No: 6 |
| OA_32 | 25515 | C to T | T | SEQ ID No: 7 |
| OA_46 | 34419 | T to C | T | SEQ ID No: 8 |
| OA_65 | 59661 | C to A | C | SEQ ID No: 9 |
| OA_70 | 59968 | C to T | C | SEQ ID No: 10 |
| OA_100 | 73821 . . . 73842 | Del to Ins | Del | SEQ ID No: 11 |

TABLE 4
SNPs that occur within or close to exons of the GTOA sequence
and which may have functional consequences

| Feature ID (see | | |
|---|---|---|
| Table 2) | Nucleotide Change | Putative Function |
| OA₋21 | UTR C to T | may affect mRNA stability. |
| OA₋22 | UTR A to C | HIS/GLN (may also affect |
| | | mRNA stability in other |
| | | transcripts) |
| OA₋23 | UTR T to G | LYS/THR (may also affect |
| | | mRNA stability in other |
| | | transcripts) |
| OA₋24 | UTR G to A | PRO/PRO (may also affect |
| | | mRNA stability in other |
| | | transcripts) |
| OA₋28 | EXON 12 T to C | ILE/THR |
| OA₋30 | EXON 11 C to T | LEU/LEU |
| OA₋41 | EXON 8 T to C | LEU/LEU |
| OA₋42 | EXON 7 T to C | ASP/ASP |
| OA₋47 | EXON 6 A to G | ASN/SER |
| OA₋56 | EXON 3 A to G | LEU/PRO |
| OA₋57 | INTRON 2 G to A | may affect splicing exon ⅔ |
| OA₋65 | INTRON 1 A to C | May affect splicing exon ½. |

### EXAMPLE 5

### Assays of GT Activity and for Detection of Inhibitors of GT Activity

### Preparation of Crude Yeast Membranes

An over night yeast (Saccharomyces cerevisiae strain Y01759—available from EUROSCARF, FrankFurt, Germany) culture grown to stationary phase was diluted back in to 1 litre appropriate yeast medium to an OD₆₀₀ₙₘ=0.2 and incubated at 30° C. shaking at 220 rpm until an OD₆₀₀ₙₘ between 1.5 and 2 was reached. The cells were pelleted, by centrifuging at 4000 rpm for 10 min at 4° C. in a Sorval RC5C centrifuge and SLA3000 rotor. The medium was poured off and the cell pellets stored at −20° C. until ready to proceed, the cells were thawed in a 37° C. water bath for no more than 2 min and enough cold lysis buffer (50 mM Tris buffer pH 7.5+1× Complete EDTA free protease inhibitor tablet (Roche 1873580)/100 ml buffer) added so that the cells were at a density of 1×10⁹/ml, based on an OD₆₀₀ₙₘ=1 being equal to 2×10⁷ cells/ml. An equal volume of acid washed glass beads 425-660 microns (Sigma G8772) were added and the cells lysed by vortexing at full speed for 2 min. The cell suspension was cooled on ice, re-vortexed for 2 min and returned to ice. The glass beads were filtered out by passing through a single layer of miracloth (Calbiochem 475855). The filtered suspension was transferred to a 50 ml tube and centrifuged at (1200×g) 4000 rpm at 4° C. for 10 min in a Sorval RT6000B centrifuge. The supernatant was removed and centrifuged at 100,000×g at 4° C. for 90 min in 14 ml Beckman ultraclear tubes (344060) filled to within 0.5 cm of the top. The membrane pellet was resuspended in 0.5-1 ml 50 mM Tris buffer pH 7.5+10% glycerol and the protein concentration measured using the Biorad Protein Assay Kit (500-0006). The membranes were snap frozen and stored at −80° C.

### Preparation of Endoplasmic Reticular Yeast Membranes

This was carried out as above for preparation of crude yeast membranes, except for an additional spin as detailed below. The supernatant from the centrifugation at (1200×g) 4000 rpm was removed and re-centrifuged at (10,000 Xg) 9200 rpm in Sorval RC5C centrifuge and SS34 rotor at 4° C. for 15 min. The supernatent from this additional spin was transferred to 14 ml Beckman ultraclear tubes for centrifugation at 100,000×g. Proceed as above.

### Preparation of Lipid Linked Oligosaccharides

Lipid linked oligosaccharides were prepared as detailed in Kelleher et al, (Glycobiology vol 11(4):321-333, 2001). Volumes were scaled down appropriately if less than 10.5 litres of yeast culture was used. Solvents were evaporated under vacuum in a GeneVac H-4 SeriesII Centrifugal Evaporator.

### Synthetic Substrates for Glucosyltransferase Activity

GlcNAc2Man9(a1-3)G1c1 was custom synthesised by Dextra Laboratories, UK (Berkshire RG6 6BZ)

The GlcNAc2Man9(a1-3)G1c1 oligosaccharide can be utilised as a synthetic substrate for GT in the assays developed for measuring activity of GT.

### Measuring Glucosyltransferase Activity by Filtration Assay

Glucosyltransferase enzyme activity was measured by incubating varying amounts of yeast membranes (0.25-20 ug) with 2 μCi Uridine diphospho-D-[6-⁻³H]glucose (Amersham TRK385), 10 μM cold Uridine 5′diphospho-glucose (Amersham U4625), 60 μg/ml deoxynojirimycin (Sigma D9305) in 100 μl assay buffer (50 mM Tris pH 7.5, 1 mM magnesium chloride, 1 mM manganese chloride) for 90 min at 37° C. The membranes were filtered through a GF/C filter plate (Life Sciences 6005174) using a Packard FilterMate Cell Harvester and washed with 6×20011/well assay buffer. The filter plates were dried at 37° C. for 60 min, 25 μl Microscint 40 (Packard 6013641) added, sealed and counted using a Packard Top Count NXT.

### Measuring Glucosyltransferase Activity by (SPA) Scintillation Proximity Assay

Glucosyltransferase enzyme activity was measured by incubating varying amounts of yeast crude membranes (0.25-20 ug) with 10 μCi Uridine diphospho-D-[6-⁻³H]glucose (Amersham TRK385), 1-10 μg synthetic substrate (Dextra Laboratories MC1131) or 5-50 ul LLO, 60 μg/ml deoxynojirimycin (Sigma D9305) in 100 μl assay buffer (50 mM Tris pH 7.5, 1 mM magnesium chloride, 1 mM manganese chloride) for 90 min at 37° C. 0.5 mg Wheat Germ Agglutinin PVT SPA beads (Amersham RPNQ001) were added and incubated at room temperature with gentle agitation for 120 min, the beads were floated by adding 50 μl 7.5M caesium chloride and incubating without agitation for 120 min, the plate was then counted using a Packard Top Count NXT.

The above GT activity assays and oligosaccharide substrate can be adapted to screen for compounds capable of modulating GT activity.

1. A method for the diagnosis of a polymorphism in GT, which method comprises determining the sequence of the human at one or more polymorphic position and determining the status of the human by reference to the polymorphism in GT.

2. The method according to claim 1, wherein the polymorphic position is selected from the group consisting of: 5283, 21037, 21134, 25515, 34419, 59661, 59968 and 73821.73842 (each according to the position in SEQ ID NO: 1).

3. A method for assessing the predisposition and/or susceptibility to develop osteoarthritis in a human, which method comprises:
i) determining the sequence of the nucleic acid of the human at one or more of positions: 5283, 21037, 21134, 25515, 34419, 59661, 59968 and 73821 . . . 73842 (each according to SEQ ID NO: 1), or a polymorphism in linkage disequilibrium above D′ 0.9 therewith; and,
ii) determining the status of the human by reference to polymorphism(s) present.

4. The method as claimed in claim 1, wherein the presence of a cytosine at position 5283 and/or a thymine at position 21037 and/or a thymine at position 21134 and/or a thymine at position 25525 and or a thymine at position 34419 and/or a cytosine at position 59661 and/or a cytosine at position 59968 and/or a deletion of the sequence from positions 73821-73842 (each with reference to the location in SEQ ID NO: 1) is indicative that the human has a predisposition and/or susceptibility to develop OA.

5. A diagnostic kit for diagnosing or prognosing or monitoring OA comprising, one or more diagnostic probe(s) and/or diagnostic primer(s) and/or antibodies capable of selectively hybridising or binding to GT.

6. The kit according to claim 5, wherein the primers and probes are capable of detecting a polymorphism at a position selected from the group consisting of: 5283, 21037, 21134, 25515, 34419, 59661, 59968 and 73821 . . . 73842 (each according to SEQ ID NO: 1).

7. A method for identifying a compound of potential therapeutic or prophylactic benefit, which method comprises subjecting one or more test compounds to a screen comprising a GT polypeptide and determining the ability of the test compound(s) to bind to, block or modulate the polypeptide.

8. The method according to claim 7, wherein the GT polypeptide is one that comprises the amino acid sequence shown in SEQ ID NO: 2, or is a homologue thereof or a fragment of either.

9. The method according to claim 7, which utilises a GT polypeptide that comprises one or more of the polymorphisms identified in Table 2.

10. The method as claimed in claim 7, which utilises a GT protein splice variant.

11. The method according to claim 7, wherein the potential therapeutic or prophylactic benefit relates to the treatment of OA.

12. A method for identifying a compound capable of inhibiting the activity of GT comprising bringing into contact:
(i) a test compound;
(ii) a cell membrane preparation comprising GT
(iii) an oligosaccharide, capable of allowing glucose addition by GT; and,
(iv) glucose; and,
(v) measuring the effect that the test compound has on the ability of GT to add glucose to the oligosaccharide.

13. A method of screening for a compound potentially useful in the treatment of OA, which comprises assaying the compound for its ability to directly or indirectly modulate the activity or amount of GT.

14. The method according to claim 13, wherein the assay comprises a cell capable of expressing the GT polypeptide, or a cell-membrane preparation comprising GT polypeptide.

15. The method according to claim 13, wherein the cell is engineered to express the GT polypeptide.

16. The method as claimed in claim 13, wherein the activity or amount of GT is determined by the method selected from:
(i) measurement of GT activity using a cell, cell line or tissue which expresses the GT polypeptide or using purified GT polypeptide; and
(ii) measurement of GT transcription or translation in the cell, cell line or tissue extract expressing the GT polypeptide.

17. A method of treating a patient suffering from OA comprising administering to the subject in need of treatment an effective amount of a small molecule drug acting on the GT protein or an anti-sense oligonucleotide acting against the GT mRNA.

18. The method as claimed in claim 2, wherein the presence of a cytosine at position 5283 and/or a thymine at position 21037 and/or a thymine at position 21134 and/or a thymine at position 25525 and or a thymine at position 34419 and/or a cytosine at position 59661 and/or a cytosine at position 59968 and/or a deletion of the sequence from positions 73821-73842 (each with reference to the location in SEQ ID NO: 1) is indicative that the human has a predisposition and/or susceptibility to develop OA.

19. The method as claimed in claim 3, wherein the presence of a cytosine at position 5283 and/or a thymine at position 21037 and/or a thymine at position 21134 and/or a thymine at position 25525 and or a thymine at position 34419 and/or a cytosine at position 59661 and/or a cytosine at position 59968 and/or a deletion of the sequence from positions 73821-73842 (each with reference to the location in SEQ ID NO: 1) is indicative that the human has a predisposition and/or susceptibility to develop OA.


SEQUENCE LISTING (11 sequences)

SEQ ID NO: 1 - DNA, 83798 bp, Homo sapiens
ctaccccggcctaagcggtcagctttctcctcctcctctgcgcgccggactcacgggcacggcgcagcgcggtgcagcgcccaaacgcttcccggtacgtgttgctgcgggaacccggcacgcgaagttccgggctactaagtttcactttgctcgtccgcccgcgctaattctctggtgggagtgggattcacgggcctggtaacgcggggtcgaggagtaccccaaacgagccaaactgggcgaagactaggagatactgattttcttttctcccaacttccccaaccagaattgcctctccctcccctgccccttctggaaagatttatcttttcatccaatacccacaaatgggcatgatgcgtagttttgataaacgtaattaattgaaattccataaaagctagttttcgttttctgagttttcagtcgttcattcgcatagacactccctccaaagcctaaaattaaggtctggccagcaacaaaagaatttagagaagcccaggactggatcgttttagtctattgttgaaattcagattcggaggcagcaccctaaaatgtttcacctctgggtcttcacgtcgttacacatggctgagtgaggaggtgttccctaaaggtttgagtaatgaggccaggcgccgtcactcacgcctgtaatcccatcactttgggaggctgaagtgagcggatcacttcaggagttcaagaccagcttggccaacatggtgaaatcccatctctacgaaaaatacaaaaaattagctgggcgtggtggcgggtgcctgtagtcccagctactgggaaggctgaggcagcataatcggctgctcgggaagctgaggggggagaatcgcttgaacctgggaggtggaggttgcagtgagccgagatcgcaccattgcattccagcctggtcaacaagagcgaaacttcgtctcaaaaagaaaaaaaaaaaggtttgagtaatgaaagaaggaatttataattctgttaagcaattttccagtctctgctgtcaggcctctcagccgaagctcaaccattgtaacccctgtgacttgcacatatgcgtccagatggcctgcgggagccaagaagtctggggcagccaaaaacccacaaaagaagtaaaacagccacttcctgccttaactgattaacaaacattccaccattgtgactggtccctgccctaccttaactgatcaaccatccttgtgacattcttcttctggacaataagtcttatgatctccccatcatgtaccttgtgaccccctcccctgctaacaatagataacaaccaccttttactgtaattttccattacctacctaaatcctataaagcaaccccttccccatctcccttcattgattctcttttcggactcagcccacttgcacccaagtgaataaacagccttgttgctcacacaaagcctgttggtggtctcttcacacggatgcatttgacatttggtgctatgacttggatcgggggaccttccttgggagatcaatcccctgtcctcccgctctttgctccgtgaggaagatctacctacgacctcgggtcctcagaccagcccaaggaacatctcaccaattttaaatcgggtaagtggcctctttctactctcttcttcaacctctttcactatccctcaacctctttctcctttcaatttcggcaccacccttcagtctctcccttcccttaatttcggttcctttccctgtctggtagagacagaggagacccgttttatctgtggacccaaaactctggcgctggtcatggactcgggaagactgtcttcccttgtgtttagtcactgcagggacgcctgcctgattattcacccacatttcagaggtgttcgatcactgcggggacacctgttttgatcttccaccttggtggcaagtaccacttcccctgggtggcaagtaccaccccccctctctccatgtctcaacctcctcctttctctaaacttacctttttactatgggcaaccttcagccctccattccttcttcttcctctcttagcctgtgttcttaaaaacttaaaacctcttcaactctcacctgacctaaaacctaagtgtcttattttcttctgcaacaccacttggccccaatacaaacttgataatggctctaaatggccagaaaagagcactttcgatttctccagcctacaagatctagataatttttgtcataaaatgggcaaatggtctgaggtgccctacatccaggcattttttacacttcgttccttccctaatctctgttcccagtgcgacttgccgcaaatcttccttctttccctcccgcctgtcccttcagtcccaacctgaagtgccggtgagtcttttgaatatttcttttctaccgacccatctgacttctaccctcctccctagactgctcctcctcaggtcgctccccaccaggctgaatcagcttccaactcttcctcagcctccacttccccaccctacaacccttctatcatctcccctccccacacctggtccagcttacaatttcgttctgcggcaaatactcccccacctgcccaataatttcctcttcaagaagtggctggaactgagggcatcgtcagagtgcatgtaccattttctctatcagacctttcccaaattaatcaacacttaggctccttctcgtcagaccccaccaaatatatacatgaattccagtatttaacccagtcttacaatttaacctggagtggcttaaatgtcatcctaatttctaccctctccccagatgaatgggaaagagtgtttcctctagcctagtcccacacagacacccactggcattatgagccaggcctccaggaaggtattacagacggttccctgagaagatccccagtggcactatcaggcaggttcctcaggtatagctaggtgagattacatgatttcctgcctagttgaagggcttaaaaaggcagcatacaaatctgttaattatgacaaacttaaagaaactacccaaggtaaagatgaagacccagcccagttcatggcctgtttggtggctacccttagatgctttatagccctagaccctgaagggccagaaggctgtcttattcttgatatgcatttcatcacccagtcagctcctgacatcagaaaaaagcttcaaaaatttgaatctggccctcaaaccccacaacaggaattaatcaacctcgccttcaatgtgtacaataatagagaagaggcagccaagcagaaacacatttctgagttacaattacttgcctctgctgtgagacaaaacccagctgcacctccggcacacaagaacttcaaaatgcctaagctgcagtggtcaggctttcctacaggacctcctccctcaggatcttgcttcaagtgccagaaatctggccagtgggccaaggaatgcctgcagcccaggattcctcccaagccatgttccatctgtgcagtgacccactggaaatcagaccgcccagctcacccggcagccacttccagagcccagcccctaaagctctggcccaagggtctctgactgactccttaccagacctgctcagcttagtggctgaagactgacgctgcccaatcaccttggaagccccctggaccatcatggatgccaagcttcgggtaactttcacagtggagggtaagtctgtcccctttttaatcgatacaggggctacccactccacattaccttattttcaaggacctgtttcccttgcccacataaccattgtgggtattgacagccaggcttctaaacctcttaaaactccccaactttggtgacaacttgtacaacattcttttatgcactcctttttagttattcccacctgctcagttcccttattaggttgagatgttttaacaaaattatccaccaccctgactattcctggacgacagccacatctcattgccacccttcttcccaacccaaagcctccttcacttcttcctcttgtatccccccaccttaacccacaagtatgggatacctctactccctccctagcaaccgatcacacacccattactatcctattaaaacctaatcactcttacccccctcagtgccagtgtcccatcccacaacagactttaaagggactaaagcctattatctctcgtctgctacagtatgggcttctaaagcctccaaactctccttacaattcccccattttacctgtccaaaaaccggacaagtattacaagttaattcaggatctgtgccttatcaaccaaattgttttgcctatccactctgtggtgcccaacccgtacactcttttgtcctcaataccttcctccacaactattccgttcttgatcttaaagatgcttttttcactattcccctgcacccctcgtcccagccctctttgcttttacctggattgaccctgacacccatcagtcccagcaacttacctgggctgtactgccacgaggcttcagggacagccctcattccttcagccaagctctttctcatgatttactttctttccacccctctgcttctcaccttattcaatatattgatgaccttctactttgtagcccctcctttaaatcttctcaacaagacaccctcctgctccttcaacatttattctcgaaaccattatataaactcagaaagggaaacctagccaaccccacagatcctaaatcctttccccactcctctttccattccttgaaaacagctctagaaactgctaccacactagctctccctgactcatcccaacccttttcattacacacagctgaagtacagggctgtgtggtcggaattcttacacaaggaccgggaccgcgccctgtagcctttttgtctgaacaacttgaccttactgttttaggctggccctcatgtttgcgtgtggcagctgccaccgctctaatacttttagaggcccttaaaatcgcaaactatgctcaactcactctctacagttctcataactttcaaaatctattttcttcctcaaacctgatgcatatactttctgcccctccagctccttcagctatactcactctttgttgagtctcccataattaccattgttcctggcccagacttcaatccggcctcccacattattcctgataccacacctgacccccatgactgtatctctctgacctacctggcatttcaacttctgtcacaaccttccattgtctttctaatgactttcctgctagcattacagatatatcacaaactttatcagtccttcgggcccaggtcggttccttagtcgcagttgtcctccaaaatcaccaaggccttgacctactcactgctgaaaaaggagaactctgcatatttttaaatgaagaatgttgtttttatctaaatcaatccagcctgctatatgacaacataaaaaagcttaaagttagggccccaaaactcgccagccaggcaaataattatgctggacctacctgggcactttttaactgggcgtcttggctccttcctactcttagtcccctggtacctgtttttctccttctcttatttgggccttttgtctttcatttagtccctcaattcacacaaaaccgcatccaggccatcaccaatcattccatactgcagttgctgcttctaacaaccccatggtactatccggtgccccaaaatctccccacagcctaaacttctattcctcgtcctattatactccttctcacctttaggactaaaccagtgatgaactcctggcaacatccagacaagaccaagcttctgtgcaacaccccctccaaacggctgtccaaacatacttccaggcctgcttcacccgttcctctaaaccataatctctgaggacctgctgtctataaaaacaacttccttcaggccttgtcctccaaaatgttccccacccccaaaatcaaagacctctgaattaaaaatctaaaatgcctggggtggcagctgtcaaatccttgcccatggggtcctcaggcctactcatatagaaatgaccagcaatatgcatatagaggaccagttccacatatctggcactcaggtcaacatccaggacgacacatggtctgtcattggttattagaaggcctaggacatttctctctttgccagccccaacttattccaggagacagggactattgtacttactgtctttcctgtatagggtctgcagtaaggactattgaactcctccattcaaaacaccactcacacctttgggaaaaaagtaacataatagacttttggctcgatgccaggggatcctattcatatgatggcaaatggggacaaaaggccttggtatataaaacgttattcctaccttggcctaaaaactcactgccacctaccttaaagctattatgcttaattgctatttttagataatttattctactaggagaattccaagctcaaaaatatgctaactggtgccttgttagccacacaaaattgtacccgaaaactttctagaccaactcactataaaattaaattttctttaggtgtccatgctgcccctaagtcatgcctgaagcagccctgagaaacatcgcccctgccccaataatccccagtaaaaacttatattttctttatcttttcttataactttatattttataaataaaaagacaggaatgtcaggcctctgcgccgaagctcagccattgtaacccctgtgacttgcacatatgtgtccagatggcctgcaggagccaagaagtctggggcagctgaaaaaccacaaaagaagtaaaacagccagttcctgccttaacagattaaccaacattccaccattccaccattgtgactggtccctgccctaccttaatggatcgattgaccttgtgacattcttcttctggacaatgagtcttatgatctccctaccatgtaccttgtgacccccctcctctgctaacaatagataaccaccttttagtgtaattttccattacctacccaactcctatagggcaaccccttccccatctcccttcgctgactctcttttcagactcagcccacttgcacccaagtgaataaacagccttgttgctcacacaaagcctgttggtggcgtcttcacacagacatgcgtgacatctgctcacccacctctaatactgcagattgaacagttccctttggaaagcccttcctgatgttgagctaaaatgtcttcctgtcatgtccattcattagtagaataaacattttatccaaatgaggctgattaaaccagaggtttatgcaaaggtgtatgcatcaaagatagtgatttcctgagtggaagaattaaataccagctaagagtgattacataaagcatggaccttttatctctatagaaaaaacgctacctgacaatatttcaagttagaattttgcttttctttttctcaagtcagggtctcattctgtcacacagactggagtgcagtggtgcgatcatgctggctaatttttaaattttttgtagacagggtctgctatgttgcccaggttggtctagagctcctgggctcaagggatcctcctaccttggcctcccaaagtgctgggattacaggcatgaggcacctcgcccagcccaaattaggatttttctggagtgtatattagcactttcctgaaaggcagcaaatgtttcaaaagttcacaccctttaactcaataattacacatctaggaatcttattctctgaaaatagtatagcatggaaaacgttttttgcacaaatttttgtttttttaaccacagtgtttcttttaaaatgttattaaaagaataaaaacaaccaaaaatgttcctcagaagcaatatgtaaattctgaataccagataaactactaggcaagcatctaaaataaagtttacagattttcttaaaacatgatgaaatacttgtgtattcaatggatattccaaggctcagcttctataaaaaaaatctaactctgagtaaggggatccataacagaagcctagcccactgctcctcaacacataacccccacaatcttctgggcctgtttttttatgagacagaggttcactttgtcgcccaggttggagtgcagtggcgcgatgtcagctaactgcaacttctgcctccctggttcaagcaattctcttgcctcagcctccccagtagctgggattacaggtgtgtgccaccatgtcctgctaatttttgtatttttagtaaagatggggtttcgccctgttggccaggctgttctcaaactcctgacctcaagtgatctgcccacctcggcttcccaaagtgctgggattacaggcgtgagctactgcaactggccctgttttgttttgctttgtggggttttttttttttttttttgagaccgagtctcactctgtcacccaggctggagtccagtggcacaatatcggctcactgcaagctccgcctcgggttcatgccaatctcccgcctcagccttccgagtagctgggactacaggtgcccgccaccacacccagctaattttttgtatttttagtagagacggggtttcaccgtgttggccaggatggtctcgatctcctgacctcatgatctgcccaccttggcctcccaaagtgctaggattacaggcgtgagccactgcacccggctgtttgttttgtttttttgagacggagtgtcactcttgttgcctatgctggagtgcaatggtgcgatcttggctcaccacaatctctacctcctgggttcaagtgattctcctgcctcagcctcccgagtagctgggattacaggcatgcgccaccacgcctggctacttttgtattttagtagagatggggtttctccatgttggtcaggctggtctcgaactcctgaactcaagtgatccacctgcctcggcctcccaaagtgctgggattacaggtgtgagccaccacgcttggcccagatctatgatgtgcaaatattttcttccattctgtaagctgcgttttcactctgcttcctttgatgtacaggagttttaaagtttgatgtattcccggccgggcgcagtggctcacactcctagcactttgggaggctgaggcaggcggattgcctgagctcaggaggtcgggaccagcctgggcaacatggtgaaacaccgtctctactaaaatacaaaaaattagccgggcgtggtggcgggcgcctgtattcccagctattcaggaggctgaggctggagaatcacttgaacccaggaggtggaggttgcagtgagccgagatcatgccactgcactccaacctgggtgacagagcaagactccatctccaaaaaaaaaaaaaagtttgatgtagtcccatttgtctagtgttgcttttgttacctatatttttggtgtcatatccaagaaatcattgcaaaatccagtgttctgaagctgttttcttttcttttccttttttttttttttttttttttttttgagacagggtctcagtgtcgcccaggctggagtgcaatggcacaatcttggctgactacaacatccgcctcctgggttcaagtgattctcccacctcaggttcccaagtagttgcgattataggcgtgagacatcacaccagttaatttttttgtatttttagtagagacagggtttcgccatgttggccaggctggtctcaaactcctggcctcaagtgatccacctgcctcggccccccaaagtgctgggattacaggcatgagccgccacgcctggcctgaagttgtattttcttctaggagttttagatcttatagttaggcctttaatccattttgagttggtttttttttttttttttttttttttctgagacagagtcttactctgtcacccaagctggagtgcagtggcgcagtctcggttcactgcaacctccacttcccgggttcaagcaattctcctgcctcagcctcccaagtagctgagattacaggtgcctaccaccatgcctggttaatttttgaatttttaatagagacagggtttcaccatgttggccaggctgatcacgaactcctgacctcaggtgatgcacccaccttggcctcccaaagtgctgggattacaggtgtgagccaccatgcctgaccattttaatttttgtacgtggtataaggtaaggttccaacttcatcttttgcatgtggatattcagttttcccaacacagactatcctttcccccgtgtatagtcttggcactcttgatgaagatcacttggccatatacacaatggtttatttctaggctctctattctgtaccattgggtctatctatgtgtgtatgccagtaccatatcatctttttttttttttttttttttttttttgagatggagtcttgctctgttgcccaggctggagtgcagtggcacgatctcggctcactgcaacctccacctcccgggttcaagcgattctcctgcctcagcctcccaagtagctgggactacaggtgcatgccaccacacccggctaatttttttgtatttttaatagaggtggggtttcaccgtgttagtgaggatggtctcgatctcctgacctggtgatctgctgagtctgtcccgcagactctggctgagcaatggatgaaagaagtacactgacacaggtgttttgcctgacagcgcagctagggctcagcaccactgacgagagagtgcagcagccaccgagagagtgcagtcccaataagctggccctgctcgcatttatgtagtacatatttaatgacaaagggttgaagcaaacacagtttgtgggtaataaacattgttgaccccccaggtagagagcagtcctgtgctcgaatgatcaaaggttggtttctggagacagaagtaaacaaatttatctagataagtttctttacattcccttgttatctgccctttgctctcaggctccggataagaaaatttgactgccttcagccataattcccttccgaagcttttggaaaacctcccggccttccaagaaggtttgcgtctttccctataactttttcttacaacttttcccaccaccctgacagaactcctacagtgatccacccgcctcagcctcccaaattgctaggattacaggtgtgagccaccacacccagccccgccccgcccttttttttttttgagtcagtcttactctttggtgcaggctggagtccagtggcatgatcatggctcactgcagcctcaacctcctgggctcaagagatcctcccacctcagcctcctaagtagctggggctacaggcctacaccaccccagctgcctaatttttttttttttttttttgagacagagtcttgctttatcgcccaggaaggagtgcagtggcgcgatctcggctcactgcaagctctgcctcccaagctcacaccattctcctgcctcagcctcccaaagtgctgggattacaggtgtgagctaccacacccggccttcagctggctaattttttaaattttttgtagagatggagcctcactatgttgtccagaatggtcttgagctcctgggctcaagcaatccttccacctcggcctcccaaagcattgggattacaggtgtgagccactgtgctagccttatatatacactctgatgctaatcttttgttaaatatatggcagatatcttttccaactattctgcttgcctttgaatgttatttatggagtctggggtggttgtcataaagtgtaaactattggtttagtaaaatgtatcaatctttttctttgttgtctatgtttttaatattattaagaagccttgttttttctaagaccataaagatatgctcagatattttcttcccaagttttagctttgcatgtttaatccattcgaatttttgtttttgtgttgagtgtgaggaatattacacgtgaaaactggccgaacatcatttattattaaagtccatcttttccctactagtatttaatgcctcctttaatacacatcacgttctcacaaatgccatggtctatttctgaaatctactttgttccacagttctctttaccaattactatacccatatcatacaatctaataaccatagtgttgaattgttctttaatatctgatagggtgtatcatctactttttttcaaagctaatacatgttagcaattattattattactattcgtacacctttagacttccttatgattttaaaatcaccttgtcaagttatttgaaaaatctcattagggtattgattagaatttcattaaatttatagactcctatggaaagaactgatatatttaaaatgctgagtcttctgatctatgaacatggtatatatttatgcatttattcaaatttccttttatatccctcaataaaccattagcattttctctacaaaattcttgaacattttcactagtatgtctctagatatttatagtttttgttactgttgcaaatatctttatattctattacttttctaattggttaattagaatgctgctcttgtagtcagcaattttgctacactttcctattagttccaatagttttccacttgattctgttgggtttccccatatccttggcaacctttgcctgttgtctgttgccattgccgaactttaatttttgccagtcttctagtagaaatgttatttcactatggttttgcttttcctttattattagcaaagctgaatatattttcatatttattgattcttctatagtttcctcttccatgaatgaattggttgttcatcttttacccattttttaaaaatgttttaattttttttgaagagatgggatttcaccatgttgcctcagctggtctcgaactcctgggctcaaacaattcaaccgccttggtctcccaaagtgttgggattacaggcatcagccaccacacccagccaatctttttctttatggtttatagtgtgtctttaaaaattctttctggctgggcctggtggctcacgcttgtaaccccagcactttgggaggacaaggcaggtggatcacttcaggtcaggagtttgaaaccaacctggccaacatggtgaaactctgtctctactaaaaatacaaaaattacctgggcgtggggacttgtgttggtaatcccagcaacttgggaggctgaggcagaagaattgcttgaacctgggaggtggaggttgcagtgagctgagatcgcgccactgcactccagtctggccgacagagtgagactctgtctcaaagaaaaaatggcaaagaatatcaacacagtactcaggagagaaaacacaaatggctacgctgagcacagtggctcacacctgtaatcccagcactttggaaggtgaggtgaaaggatcgcttgagcccaggagttcaagaccaacctcagcaatctaataaaccatttctatttaaaaaataatgaaaatagggtcaggtatggtagctcatgcctgtaatcccagcactttgggaggccaaggcaagtggatcactggaggtcaggagttcaagaccagcctaagcaacatggtgaaaccctgtccctactaaaaatacaaaaatgagctgggcgtgatggtgcacacctgtagttccagctactcaggtggctaaggcaggagaatcttttgaatctgggaggtggaggttgcagtgtgccgtgatcgtgccactgcactccagcctgggtgaggagcaagactccgtctcaatcaatcaatcaaatacaaatggcttttaaacatataagaagtttctaacctctctcataagagaaatacaaatgaagataacagatgtcactggcaaagcttgatgacacagtaaattggcaaatgtgtgaataggcatccttatacattgcctgtgaaacaaactgatgcaacttctatcaccaatttggcaacatctatcgaaattacaactgagcatagccttcaatatagaaattctagttctggccgggtgtggtggcttatacctgtaatcccagcactttgggaggccgaggcgggcggattacctaaggtcaggagttcgagatcagcctgaccaacatggagaaactccatctctactaaaaatacaaaaaaaaaaaaaaaaagaaattctagttctattaagcatattctgagagtgaaaaaaaaagagaaattctagttctagaaatttaattcaacaacatgtgcagtcctagcacacaggcagaatggaatactgatattgatattgattgcagtgttattcatactgtcaaaaatggaaacaacttaaatgtccactaagagaaatcttgttaaataaattctggtccatctatacaatagacactgtaatgaacagactctaagatggccccaattatctctgcttcctggaactcatgctggtaatcccctcccatacatgtgggttggacatgtaagttcttctaacagaatgtggcaaaggtgacaggatgtcactttcatgattacattacacaagatcatgacatattattattttttgagacaaggtctggctctatcaccgaggctggagtgcagtggtgcaatcttggctcaccgtaacctccccctcccaggttcaagccatcctctcacctcagcctcctgagtagctgggactaccatgaccagctaatttttgtatattttgtagtgacagtgttttgccctgttgcccatgctggtctcaaactcatgagctcaagggatccatctgcctcagtctcccaaagtgctgggattataggcgttagtcaccctgcctgcccaagatcatgacagctatcttgatagccaacttccttccttgatgtctttgatgaagctagctgccatgttgtgactaccccatgaagcgggtcaattgacaaggaattgagggttggtttccacccaaaagccaacaagaaactgaggccctcagtcctacaacccatagagaattgaattctgccaacaaccgtggaagcagatcttttctcagtcaaaccttgggatgagaccacaactctgggtggcaccttgattgcagccttgtgagagatcctgaagcagaggaccaagataagctgtgcctagactcctgacttacagaaactatgagacagtaaatgtgtttttttcagccactatgttgttacacagcaatagataactaatacagataatatgcagctcttaaaagaatgatgcagttctatatgtactgataacagaataaactccaaaatatactaagcatacaaacacacacacacacacacacacacacacacacaaaagtgaggtcagcaggcacagtggctcacacctgtaatcccagcactttgggaggccgaggtgggtggactgcttgagccaggagctcaagaccagcctgggcaaaatggtgaaaccccatctctacaaaaaatacaaaaattatctggtcctggtcatgcctcctgtaatcccagctacttgggaggctgaggtggaggttgcagtgaaccgagactgcaccactgtactccagcctgggcaacaaagcaggaccctgtcaaaaaataaataataaaaaatgtgaggtcaaaagtgtgtataatgttttcatttatgtacataaaaatacttgtctatgcatagaatatctctggcatctctggaatgatacattaaaaaattggtaacaatggttgcctcttggtaggggaactgggtagctggggacctcacgaattttgctccatctacatgaaagtataactcagaaaatctgaaaaccatttgttcccatttgacttaaaatcaaattcccacatgatttgggcgctgcctacttcttcatcttcaatttataccactcttctttgctcttaggaccctctagccactagaatataaattccttgtaggcaggaatatggctttcttgtccactttttgatctccagcactgagcacaatgtataacactcaatagaaattcattaaatatttttgtgaagtgataatggatttcatttgcagttttagtagggattaagagatgtaatgtctagcaccgtacttggcacataacagtatggggttttccttctgtgtatcacttgaagggctgttatttaaatatgagtagaccttcagtacgttctgctccagaaaccagtaggagaaagtcagagaaggaaaaatactaattcatcattaaaaatgccctaacaaacagctgcacatatgggaggggctgctttgagaggtgaacactcccatcctgctttaatcagctatctttgtcagaaaaacacttaaaaaattaataaatctggtcgggtgcagtgacttatgcctgtaatcccagcactttgggaggctgaggcaggcggattgcctgagctcaggagttcaagaccagcttgggcaacacggtgaaaccctctttactaaaatacaaaaaattagctgggcgtggcggcgtgctactcgggaggctgaggcaggaaacgcttgaacccgggaggcagaggttgcagtgagccaagattgcccgggcgacagagtgagactctgtcttcaaacaataataataataaataaaaataaaaaatatgtggaatgtacttctgaaagcctttctaggactctgaaattcttataaacagtctcttttcagagcattgtacagaggtatattgagtcttgtccctttcatagtcactttatcagtacaaatatttttgttgtttttttttttaaagacagagtctagttctgtcacccaggctggagtgcagtggtgcaatctcagttcactccaacctccgccttccgggttcaagtaattctcctgcctcggcctcctgagtagctgggattacaggcacccactaccccgcctggctaatttttgtgtttttagtagagatggagtttcaccatgttggccaggctggtctcaaactcctgacctcaagtgattcacctgcctcagcctcccaaagtactggggttacaggcgtgagccaccacacccaacatatattgtttttttttaagatggagtctcgctctgttgcctaggctggagtgcagtggcacgatctcggctcactgcaagctctgcctcccgggttcacgccattctcctgcctcagcctcccaagtagctgggactacaggcgctcgccaccacgcccggctaattttttgtatttttagtagagatggggtttcaacgtattagccaggatggtcttgatctcctgaccttgtgatccgcccgcctcggcctccaacatatattgtttttgtttctgactccacagtttgaaaaaaaaattagtacctttattgacttttgtataaaatggagatgctgaatatgggacttcattgtgagaaatgaatggctatactgcttcaagcttttaaggtttacatgatcattccaatcttaactgccattagcacagtagccaatttctttggagaacaaacaaagggataggtggattttttaaaaaaccagcatcatgctataagtgtaatttcatcaaaatcttaaagagaaatatatctaaaaacaaacgttcaaactctttttaacagatgactcatgatgcaattaaaatcaaaatatgtgcataaaattgacattttaatcaaaaatacatatttttcaaaggtcattattttattggcatgttttctttcctattaactgcaaacagaagctgcatgggctatacctagggggcatttctgatactgagtgcttccttcacaaagtttcagggtggaagtctataacactatacaagtctaaaacacttaacaagaaacactataccagagttgggctagaaagacagcacatcttcctagtagttcccgcccagttctggctctatgcctccacctggggaaagatacagtgaccaagaacacgaagtcccaccacttattcttttatcttcttaaattgtggcacattgcatatacattaattagtaggtaagaaatgagttatgtatcttatttgtttacattttgataatactatcttgttcttaaactttgcttaattcccaatttcaaaatattttgaaacagcatttcttttttttttaagatggggtcttgctgtgtcacccaggctggagtgcattggcaggatcatggctcactgcagcctcaaatgcctgggctcaagtgattctcccacttcagcttcctgagtagctgagattacaggcacgggccatcacgcctggctaatttttatttttgtagagacaggtctcactatgttgcccaggctggtcttgaactcctggactccagtgatcctcctgcctcggtgtcccaaagcgctatgagccaccacacgccaccagcacttcatttattgactgaaatttaggaatttgttgttgaaaaccacccatatctctgtaaagttttaaaacatgattacaaagtatagtgtttggaaaagaataaaagaatcttgggctctagacaaaaagtctagattctagtctgtgttggcaccaacaaggtattcttcagagtctggagccatttccctcccggtaatataagacattgacttttttttttttttttttttttttttgagacagggtcttgcttacttccccagtctggaatacagtggtgtgaacacagctcacttagcctcgacttccaggattcaagtgatcctcccacctcagtctcttgagtagctgggactacagcgcatgtcaccaggcttggctaattttttgattttttgtagacaaagggtctcactatgttgcccaggctggtcttgaactcctgggctcaagtgatctgtctgcctcaggctcccaaagtgctgggattacaagtgtgggtcactgtgctcagctgaactgctttaaggctatatttggactttgaagtcaaatgaaaatgtatatgaagccatgtgaatatatgaaacagatcaaagctatttactatggacagagcccagggcctctgaggcaacactgaagaacctccagtgctctacagaacattctggaaatccctacactagatgagctcttcccgttctgacatcacatcactttcataataacaaccaaaaaagtaaatttggtcaggttgaaatccatccaaattcatcttctacttttttttttttttggcttggacaacagcaaagacgtgaggaactatactggtcctttgtgttaatgcttgtactagtaaaatatttaggttttaatcatggctggtttggtgtgcttcattcattcattcaactgtgctaaaaactggggctatgaagacaagtgtggtctcaaactgctagtctcctggggaaaagaggtaaacaagcataacacagtattacagcagttaatggtatcagcagactcctggcacagtgggagcaactcaattctgcaggaggagggaaggtagagggataggaaaagattcacataaatagcaatgctattgaccgagcacttaaaatgcatcaggcagtgttcttaatcttttatgtaatcattttagagatgagaacattcagataaaagatggttaaataacctgcctacagattttaaaaaaaatgatggaggcaagatttacaaccaggacatacgactttaaggccctactcttaatcactacaccattctgcctttatattgacagacatggtagcagttaagctgaatcttgaagaggttcatataatcagatgacagggtagaagtcagcagagaggagaggaagcagcatgaatgaaagggaacagaggtacaagacagcatggcacacggggagtttgaagttgttaggccagctggaacacagggtggaggagagtctgtgggcaggaaaacaggttagaggcgctacgcttccctccccctttggcaagatccccatctttgtctctgcaataacagaccaaaaagaagtcttagaaaatggagtagacggaagagatggaataactggattccatctcagaatgccattgaccttggagtctctctgatcttaggggataatgggtaaataaaatgtatgaattctgcctgagggctgaaaaaaaatagaaataaaataaaatgtgtgaaagttcctactgttgactagataaacgttggctgaatctgaatgtgtggcagatattacacaaattggggtcctggataaaaagcgtatatgaagtactcttaaggaagggaaaggataagaaccagactgcaaaaatcacttcagttcctgcgcatgatgaaaggtttaagtggctgccttataaaaactgcagtgcttgctgttagaataaatgagctgcacgagatatccttaaatcaatgtaaagtagctgtggtttgtagtttctgctgcctggccaaaccagtaccttaactactccccttggctttgtgagtattgtcactgtataatttcaaacaaaaaaaatctcgaacaaggtgtacagaatggggacagtacaccatctgcctgtgtacacgccaagaggaaaatgtaggagctggtaacgatccagaagtttttttgtgcatacaaaatagcatttcatttatatttccttagcacaggattctaattatgctttatatcctcacaatggttagcataggctacaaataattatgcggttaatcagattccctgtttggcttgagcaagcacagcagtctgaacgatctcagacaattttaatagggaaaggtgagtttttttcagatgtgtttcagtgcttatggagggccagtggcttctcaccatggtgacaatccggttgctaggctgggcaagaccaagtgtggaagaatggtgctctgaacagttctactgaaacaaatggctgatgccactggcaactttcttcaagttctggttaatttcccatgaaataattgcttggcacatatctaagcagttcctttattcattgtttctttgtcttgccaatagcagagtcaatcaatactgaaacatacagtttgaaccaagcatatgtgatgcctactgcacaatacactgaggttagtaacaaagggatgaaggggtacttcaccttccaggaggtgaaagggaatacaaattcacagcagacttccagaggccccaggccaagcaggtagaaagtttccatccaattaaaaagaggtttttcttttctgaaggaaaaaagagagaaaagtctgattttagatgagtcatcctggtttagcaaacatttcctagctaaagcttccccatctgcactgtcttgctgtcaattattatagccaaggccagagtcaagagtatattaaatttcacatttagggccaagtgcagaggctcatgcctgtaatcccagcactttgggaggccaaggcaggtggatcacctgaggtcaggagttctagactaacctggccaacatggagaaaccccatctctactaaaaatacaaaaattagccgaccagggtggcacaggcctgtaattccagctacttgggaggctgaggcaggagaactgctttaactgggttgcagtgagtcgatatggcaccactgcactctagcctgggcgacagagtgagactctgtctcaaaaaataaataaaaataaaaaataagtttcacatttagaaaaaaatgataattatatacgttatattatctcaacaatctaaatttactccaccattgacctatgctgttatttagctcaagctgctcagagagaattagttctgcatttgaaagaggtaaaggggttgggtgctgtgacattggaataagaacatgagaaacaatatagaattgaagatttaaaaaaaattttctttttgagatggagcgttgctctgtcacccagactggagtgcagtggtgcaatctcagcttactgcaacctccacctcctgggttcaagcgattctcttgcctcagcctcccaagtagctgggattacaggcgcctgccaccatgcccagctaatttttgtattttctatagagacaaggtttcaccagatttttaaaatattttttattttggtaaaatatatataacatgaaatttgctatttgtttaaccatttttaagtatacagtttgatggtattaagtagatcaataatatgcgcccatcgccactatccaccttcaaaatattttcgtgaccccaaacagaaactctggaaccattaacaataacttcccatccccctgttccctcaacctctaatctactttctgtctctatgtttgtttattctagaatttcctatcagtggaacgatatgatatttgttctattctctcgtttctttcacttagcatgttttcaagattcattcatgtagcatgtattggaacttcattcttttctttggctgaataatccactatatgtatatatcacactttatcatgtatttgttgatggacattgggttgtttccatcttttagctagtatgaataatgctacaatgaatactgatgtacaagtatctgttgagtccctgtttccattcttttgggtataggggtggaattgctgggtcacatggctataactgcattttaacatagttaattatataagtttttatggacacagttatgaaccataagcaagctttaaaaaattacagttggccgggcatggtggctcacacctgtaatgccagcactttgggaggccaaggacggtggatcacctgaggtcaggagtttgagaccagcctgaccaacatggagaaacccatctctactaaaaatacaaaatcagctgggtgtggtggcgcatgcttgtaatcctagctgctagggaggctgaggcaggagaatcgcttgaacctgggaggcggaggtttcagttagctgagatcgtgccattgcactccagcctgggcaacaagggtgaaactccgtctcaaaaaaaaaaaaaaaaaattatagttaacattacatacagtttagaagagaaaaagtttccatctataattctgtagtaaaatttgtacatagttgggtgcggtggctcaacgcctgtaatcccagcactttgggaggccgaggtgggtggatcacctgaggttgggagttcaagaccagcctgaccaacacagagaaaccccatctctaataaaaatacaaaattagccgggcacagcagctcacgcctgtacttccagcactatgggaggccaaggtaagtggatcacctgacgtcaggagctcgagagcagcctggccaacatggtgaaaccccacctctactaaaaatataaaaaattagccaggtgtggtagtgatgcatacctataatctcagctactcaggaggctgaggctggagaatcgcttgaacccaggaggcggaagttgcagtgagtggagatcgcgccactgcactccagcctgggcgacaagagtgaaactccgtctccaaaaaaaaaaaaattagctgggcatggtggcgcatgcctgtaatcccagtaacttgggaggctgaggcaggagaatagcttgaacccggtaggcggagggtgtggtgagccgagatcacgccattgccctccagcctgggcaacaagagcaaaactccgtctcaaaaaaacgaacaaaaaaaatttgtacatagtatcttttagttttttccatatctatctgtatttaatagttttattcataaagcatattttacattcagtttttgagttttcatataggattttaatttaagtagtcctcctcaaatactcccccaaactaaagacacaaactatgttgtctcgactgagtgatgggattgagtgatttctccctctatttaccaattttttttgtaatgttgtcatattactgttataacttaaaaataaaatttaaagagcttatttgaccacttaggtgtaaacatttcacaagagtaagtataattttaaacatttttttgattacctgaataaagtcttcagtgacgaaatactatatatggtgaatagtaacatgagtaagattttaatgggaagttctgttaaaagaatagaaaaaaaaagataatttagctgatgacagaaagacttatgatgctgaaagtaatataagcacactgacacagctatatagtgctaataatactttttaagtttaattttacatttttgtgacatctagagatgctgagaattacaggaaattggaggttttatttaagaggatgaatgatctctatccaagagattttagagttgtttaaattagatgtattaatctgtgggcctcgatgaaaaggttgtgatagtcaaatatatttttctcagaagacgctgtttacctggtgcagtgaagagcagaggaaagagggaataatgtcctgttgtggtcagaatcagaaaaatcgaagcgtctcctgcttttcccacagacaaaaggctaaaaataaaaataatttcttaaaacaacaactgttattacacttgcaaatagcttaaataaactgcatctcctattacattagaactggctgaggcatagaagaacatgctgattttggcacaagttcaatttagttgagaatattaattttttagtaggaaattttaagcaatcaatagcatgtatcaaactgaatcaggggtatcgtaaaagaatcttgcttctggccggggacagtggctcatgcctttaatcccagcactttgggaggccgaggcgggaggatcacctgaggtcaggaattcaagagcagcctggccaacatggtgaaaccctgtctctgctaaaaatacaaaaattagctgggcatggtggcgggtgcttgtaatcccagctactcaggaggctgaggcaggagaattgcttgaacctgggaggcagaggatgcagtgagccgagatcgcgccactgcactccagcctgggcaacagagcgagactctgtctcaaaaaaaaaaaaaaaagaatcttgcttccctgagtaattgccttctccaaagacaggaatgttgtgggattgggagctgcctagtggtggcagtcagggaaacaggacttggagtttcatttccaaccctatagctcatctcttggacaatgcccttcacctgctggtgtttggtgtgaattcagactaaagacattaaaagtttttgctttactttttaatttggaaactccaagctgctaaataaacccagacaagccacatactggcctgacaaatttgcttaatatatgttcagagcttaaatgatttctcaagattcaagaaaaatataaagaaagaggctattattaccatttatttatctattgggtttcaactatgcctaaactggacacttacatttaaaatggtttttttagcatggcatagtgaaaagatcatagattcaacatcataagattcaggtcctaactctaattcttgcgacctgtatgactttggacaagtcacaaacattttgagccctagattcttcagatgttaagacagggaataagcctgttttgttggctcacatctgcaatcccagcactttgggaggctgaggtaggaagactgcttgtgcccagtagtttgagaccagcctaggcaacatggcaagaccctgtctctaccaaaaagtaaaaaaaaaaaaaagaaaggaaaaaaaagggaataatacccatttattttaactatcttttttttttttctttttttgagacagggtctcactctattgccccaggctggagtgcagtggcgccatcttggctcactgcaaccttcacctcccaggttcaaaggatcttcatgcctcagcctcccaagtagctgggattacaggcatgcaccaccatgcctggctaatttttgtatttttagtagagatgggattttgccatgttggccaggctgatctcaaactcctggcatcaagtgaaccaccgcttcagcctccgaaagtactgggattgcagatgtgagccaccatgcctggccaattatcttaaagattattgtgaggcgcaaaagaaagaatgcaaaacaaaaacagttctggaaaatctgaaatgtagtacagtatcaggtattattccagaatagactaaatccttactttgactaacaacacttaacaaggaaccatgggaccttattttcagacgggatgacaccaaagcccaagttttttctttttttcatttttgagacagagtctccttcggtcacccagaatggaatgcagtggtgcgatctcggctcactgcaacctcttcctcccgggttcaagcaattctcctgcctcagcctcccgagtagctgggactacaggcatgtgccactatgcccagctaattttttgtatttttagtagagatggggtttcaccatgttagccaggatagtctcgatctcctgacctcgtgatctgcccgccttggcctccctaagtgctgggattacaggcgtgagctaccgcgcccggccagcccaggttttttctataacatactgtccctcaatgatcagatctaaaagccggctttcatggtacttaatcactacagtgcttctttccaaaaacgcagaaacagaaatagtacatgcatttttgcataccttcatgggtccaagaatctcaagttaagcatccctgtcctgtcctagtggacatctgttcctatacatctttgtttttgcctatcctggtgaacatgacaagaagggacagagcaaatagaaaataggatcattgtgaaatatgccaaaatgctcactggctgagctatctgcttacctcattgggagaattgctagaagtatggctttttcatgaacatgccacccaaacataaaggagctcaaggcacaaagagttagacatcggagaaagcctctgggcccttggggtttaaaccaaagacagaaaatagagggctagaaacaacaggcaaagataaacttcagtatcatttgaaacagactttacagaaacagagttcaggtgaatacaatttgcatcatctctgaaagacagccaatcttagacaattcttcatgaaaccgaatcattcatcactttgtgcacagtctaacaaaagttaacataaaagcaacaactcagaaaaaattggcatttgatgtgtatatatatatttttatatgtaaataaatatattttatatgtaaatatatatatatatatatatatataattttttgtttttttgagacggagtctcgctttgttgccaaggctggagtgcagtggtgcgatctcggctcactgcaacctccacctcctgggttcaagcgattttcctgcctcaacctcccaagtagctgggattacaggtgcctgccaccacgcccagctaatttttgtatttttaatagagacggggtttcaccatgttggccaggctggtctcaaaatcctgacctcgtgatctgcctgccttggcctcccaaagtgctgggattacaggtttgagccaccgcacctagccttgatgtatatatttaactcattaaaattaattctactggctgggcatggtggctcatgcctataatcccagcactttgggaggccaaggcaggcagatcacttgaggttaggagttcaagaacagcctgaccaacatagtgaaaccccatctctactaaaaatacaaaaaaaactagctgggcgtggtggtacatacctgtattcccaggtactctggaggctgaggcaggagaatcgcttgaacctgggaggcagaggttgcagtgagctgaaattgcgccactgcactccagcctaggtgacagaccgagactgtctcaaaaaaaaaaaaaaaaaaaaaaattggccgggcctagtgactcacgcctgtaatcccagaactttgggaggctgatgcaagtcgattacgaggtcaggagttcaagaccagcctggccaagatggtgaaaccctgtctctactaaaaactacaaaaaaggccgggtgtggtggctcaggcctataatcccagcactttgggaggccgaggtgggcggatcacgaggtcaggagatcgagaccatcctggacaacatggtgaaaccctgtctctactaaaatacaaaaaaaattagccgggcgtggtggtgcatgcctgtagttccagctactcaggaggctgaagcaggggaatcgcttgaacccaggaggtgggggttgcagtgagctgagatcgtaccactacactccagcctggcaacagaacaagactcccgtctcaaaaaacaaaaacaaaaacaaaaacaaaactactgcaaaacaattagctgggtgcagtggcaggcgcctgtaatcccagctacttgggaggctgaggcaggagaatcgccactgcactccagcctggatgacggagtgagactctgtctcaaaaaaaaaagaaattaattatactatcaaaattaataattaagttctgggatgtacagtctaacaacttattacatcttcataattagtttactaatagttcatttattaatgaacctaataggtcaaactacagaagtccatagatgcttttgagccaataaattagatttaccatattaataaatacttagttttattggctggaaggccatagaaactatatgtacctcataaggctgaaattttaggaaaaatgttttacctcaattaaaatatgtgaacaaacagcccaacagagcttaattatctacatacctgtactgtcatgtaattaagtggaatcaggtggttgctagcaggtttgaaacattcagaaaccaaaattaaagatgagtgtaattatcgataggattgagttgtttatattgaattctactaaaattataccatgaaattgaggtcactgcaatcactaatgaaattttagaaattaaagtttctaaagtacttagaatgaggcctctaagagcatcttactcatattaggctcatcttaggctcaaaaagcctttgtcgactctctcaactcattatcaccatcaagacaaaacatccacgctgtccagatacatttggaattttgatgatttagctaacggtgagaaaaacaaaccaaatctggatgtccctgccacaaacatttccttctttctctgcagcatgatccatgcctcttcctctctgggcttttctttttcccttcccccgggtgaacagatggtctaacagccactggcaagaatctctcacaaaggctttactggtaatccttccttatggtattcaaagttactctaagaattgctaggaaatgcttcaatctgcagaaggattacaggcatgaaatttatcctacagttggaagagcttgaaagttgaaatttaatcagccagacaaaagaaaagcagagaaaactcaagaaatgagcaccatctgttgagttcttaccaatatggcaatcagtgtgcagatgagggttgccaagggagtcactgagggaaggactgtgtgttggaactgctgaaccaaaccacttgtcattgaggccttgggaatattgttgggatcaagaaatttcaatttcaaacctattaaacagatattttgttttgttttatttttatctttattactgagataccatatattaacaatcaattccttgaggaataaaaagtaagctactaaatcttattgctgcttaaaggctaagatttcatgaatttaatcctatactattctacccaaaatgcctctggtcttaagattataatatataaagcagctctttgcattggaacagggaatttgtgcagcttataggaaacttgctggcaggcagcacaaatggtaatctgctctcacctctgaaagcaagcttctagaacagtctgagcatgtcatgaaggaactttgaatccttcatctcctatgggagaaaccctctattcctaaacatagtatccagtgtcttccatgatctcatcgccccttcccccctgttcaatctcatctttactttgttaaatgcggcttggccacaatgaagtaactgtgagtcctcaatataccactctcaacacctgtgtccctctgcccaggcaactccctttgccaaattaacttctaattcaatcacctgagaaagctcctaaatcaataagcagaactgattttcttgtgagctcactttttttttttgagatggagtcttactctgtcacccagagacagaggctggaatgcagtggcgcaatctcagctcactgcagcctctgcctcccaggttccaggttccagcgattctcctgcctcagcctccccattaactgggattacaggcatgtgccaccatgcccggctaatttttgtatttttagtagagacggggtgtcaccatgttggccaggctggtctcgagctcctgacttcaggtgatccgcccatctcagcctcccaaagtgctaggattacaggcctgagccaacgtgcacagctgagtgagctcactctaacagtgttcaccacaaggcactgcacttgtttatgtgctcatttcctctactatactgtattccttgaaggcaggaatgatgatgagttcatttctatccccagtacataggagctcaataaatacttggttattaaataaatcaattcagagaatgacaatacaagccccaacaagctctggctaataggacagtcagagacagtttgagcacctatagggcctgtcaaatgccagtgaaaatgaatacaatgtgggaaagatgtaccaaaatattttctacttactaacctcttcccctttcccctttggcctgctgattctctcttacagctaataactatctccctaaaacaaagttgtctaactctgacactgactttccatgtaaaacatcttcaaagacacaaacatacaagtgagggtacagtacagcttcctgactcagacaaggcaactggcccttgtacctcatactatgctggctgtttaaagctttttttgaaatatgaatgttaggaattactctgtaagaatgccatgtacatcaaggctagattcacagtgagcaacacggtgacacaatgtataagtcatcacataggcagtgtatgatctcctccaagtacttatcaaaaacagtaatagtggccaccatgcatggagcgtctaccatgtgcccagggctctgcacttactacttttagccccctgaggtacagagatggagtaacctgcccaaagtcacacaactaataggtggtaaaactgaactcaggtttgtctggctccaaagtctctgctactgataacctagtactttttaaattttgcttctgttcctcacaacacactgtataggtacaggtaaagtactagtatgtctttcgtatagtgaggaaagttagacgaaaggaagcaaatgacttgtcaatgccacacactgcaatagcaatttacacaggtgagcctccaaaccaagtcatcagactctaggtctggagtccacaaactataggccaacagccaaatgtagcctgtgcccagtttttgcacagcactgaagtatgaatggtttttacttttataaggttttaaacaaatgtgtgcatacacaaatacagacacagacacacatacacacacacacactgtgtgtggcctgcatagcttaaaatattatctggccagttacagaaaaagtttgctgaccaccactcttgatcatgccattttcccaccaagctagattgtattcacaaatgagagactgtgttatccagagtgagttatttttagtagaaccatctcaaaccataacttatttttcattacgacccaattttaaaaaccatagcctgggtttttcatttcttctcacctcatgttaaaatgcagggttattttccttactggtttaataccgagagtcacaacttacatattcttctttcttgtatgtttaggtttaaaagtggggtaggccaggcatggtggcttgtgctggtaatcccagcactttgggaagccaaggtgggaggatcatttgagaccagaagtttgatcccactctgggcaacatagtgagaccctcatctctaaaaaacaaaaatttaaaaatcagccaggtgtggtggtatgcacctgtggtcccagcaactcaggaggctgagacaagaggactgtttgagcccaggaatttaaggctgcagtgcactatgatctcaacaccattgcactccatcctgggtgacagtgagaccctgtctctaaaaataagtaggggtttgaataggaatggtgagaaggggggaaaaaaagcagggttaactgctgctgctgaatagttttttaattaaggcaaaaattacctttatagagtgtctacatattttattttttccccaggaaaggatcaataacctcattaccacaaggagcttttataaggaaaacagtctgtttactgcactaaagcctcagtcccaagaactgctccccctgttcatcctgaaagctgaaatgcaggactggagaacaaacaattcagccacattcaaaacacaaaggtttttccatttctccatgtgcctaagtcctttcaatattcagagtctgagtaaaaaatgctcgctaccataccgatgacagacagcactttgtccaaagcattgtacaaagcccagaagtttggagcccaatatgcatgacagaggcccctcttgaaaggaaagagtcgggaaaagacttgaggcagctgattctgttgaaaagagaaatgaaactgattaaacagtcatcaatctttttcaaattcaaaaagagaactagggaactctctatggtatagtgtgtagaaagttatggggtctgggttctagtaagtaagtggttatatgctcaccacaatgtaagttcacagaaccttcttttggtctcactttgtccactgtccagtgggacaatatacataaaaatactctgaatacccgtccatactattataataacagaagaatttacaagctacaaagcagaacacagactgcagcttatccatgtctactagggtgaaggggagagctgggaaactacaaaagatgtgacgcgtttcagttttagtaagaattaaaattaattagttgatttcagggcagggaagaagaggaagaaagaaaacatctgaacatctgttaaattctatgcactttagaatgtctgctgtccatgtaagactgcaaaattattgattttgttacaaatacattttatcaagtaagtaaacttacaagtacagaatacacaaataatgaagatcaattctgtgtttgtgtatgtgtaatgtacagttaaacatatacatgtatatatatatacatatttgaatgtgcacaaaaatgtctgaaagaatactaccttatcgggccaagagcggtggctcatgcctttaatcccagcacgttgggaggctgaggcaggtggatcacctgaggtcagaagttcgagtccagcctgaccaacatgttgaaaccctgtctctactaaaaatacaaaaattagccaggcatggtggcatgtgcctgtaatcccacctactcaggaggctgagacaggagaatcgcttgaatccaggaggtggaggttgcagtgagccgagatcacgccactgcattccagcctgggcgacacagtgagactccatcttaaacaaaaacaaaaaaaaaaaaggaatactgccctatctttggggagtcacgttgggtaaggagtaagaaaaaagtagcttttacttattttacatattcctggctttattaggttttctaaacaccaacaaagaacatgtattaattgaaaaaaaaaaaaaaaaaggcttaccaaggccaggaaaggacccaatgaaagagcagaaactaagaaaacaaccagtcccagggaaataacacgaacaaagctgaaactcttccatcgaatagacccatctacagaaaaggaacattatcagtaaccaggaagatggaaaggaacattaaccataacctatatcatgtgctaaaactagaagtatcccacaatagtataccaatctattcatgatgtggcaagagcagagaaaagcgagaatgggtaaagggcttacaggattagcagctgagatatctgctgtgcttcataaaaccttcttatcaagcaagccaccaagtcaacacagtaagggaaaaatcgaaaatgtttttgctattattaccaaaacttgcctggtttatttgcagtgaaacagtaggatcgcagcagatatacaccataagctggtgctacatagaggtagatatgcttgaaatgtaggagaacagcaaagagaaatgctccttccatatgccttttctaggagatttaaaagggaaatatcactttaaaattcagggtaaatgaaatgcaataatgtggtattctagattgaatcctggaacagaacaaggacattaaaggaaaaacgggtgaaattcaaatgcatcaatattgctttcttagtcttgatgaatgtaccatggttatgtaaggtggtaatgggggaaaatgggtgagggatatataggaactctctgtactatttctacaacttttctggaaagctaaaattatcctacaaaaaggttatttaaaaaactaaaattaggctaagtgtggtgactcacacctgcaatcccagcactttggtaagccgacgtggaaggattgcttgaccccaggagttcaagaccagcctggacaaaatagggaggccctgtctctacaaaaaaataaaaaataaaaaaaattagctaggcgtgatggcttgcacctgtagcaccagctactctggaggctgaggtgagagaatctgctttagcccaggaggtggaggctgcaatgagccatgatgacgacactgcactccaacttgggtgacagagtgagaccctgtctccaaaatgaataaataaataacaaaaataaacaatacctacttttttaaatttttattttattttattttatttgagatggagtctccctctgtcttacccaggctgaagtgcagtggcacaatcctgactcactgcagcctccgcctcctcggttcaagcaattctcctgtctcagcctcccgagtagctgggactacaggcgtgtgccaacacacccggctaatttttgtattttttagtagagacagggtttcaccatgttggccaggctggtctcaaactcctgacctcaggtgatccacctgcctcggcctcccaaagtgctgggattacaggcgtgaaccgccatgcttggcccaaggtaaatattctataaccagtataaggaaaacataggcaagtgattccagctagctttttttttgagacggagttttgttcttgttgcccaggctggagtgcaatggcacggtctcgtttactgcaacctccactttccgggttcaagtaattctcctgcctcagcctcccaagtagctgggattacaagtgcccaccaccacgcctggctaatttttttgtatttttagtagagatggggtttcaccatgttggccaggctggtctcaaactcctggcctcaactgatccacctgtctcagcctcccaaagtgctgggattacaggcatgagccactgcgaccagcccagctagcttttatactgagtctaatgtgttcataaattcactattccaacaaagatttatcacatactaagccttatgctgagatcatatcctacatgtcatcagtaataacatactgcaaactctcctttaggaattagagcccagagaattaaattaccttcacccctcacagtttcagaatagctggagttgatttccaaagaagggctgggaatgagaacatgaacaaatatataggatgctatgctaccaaaaaaatcaattgaattttatattgtagattaggtaacttatatagcactatcaggctgaaggcaccagtcactgcacttgatacaaacactacttaggaaaactaaaggtattctgatgtaaaaagaacaaacaaaggccaggcgtggtggctcatgcctataatcccagcactttgggagaccgaggcgggtggatcacttgaggtcaggagtttgagaccagcctggccaacatagtgaaaccctgtctctactaaaaatacaaaaaattagccaggcatggtggtgggtgcctgtaatcccagctacttgggaggctgagacacgagaatcacgtgaggtggaggttgcagtgaggtggaggttgcagtgagctgagatcgcgccactgcactccagcctgggtgacaatgcaagatgctatctccaaaacaaacaaacaaacaaacaaaacaaaaccagcttactataactaagaagttatagtaagaatttggctgggtggggtggctgacacctgtaatcccagcactttgagaggctgaagtgggtggatcacgaagtcaggagatccagaccatcttggctaacatggcgaaaccccgtctctagtaaaaatacaaaaaattagccgggagtggtggtgtgcacctgtggtcccagctaactggaggctgaggcaggaggactgtttgagcccaggaggttgaggttacagtgtgctgtgactccaccactgcattctccagcctgggtgacacagcatgacgttgtcttcaaaaaaaaagaaagtctgaaatctatccacctcacaggaatttcggaaggattaaatgagataacagtcctgatgttctgcaggggaaactgccacagggaccatgaaaggccagtttccttctctcatcaggtgcggtctagaattggtagctttaggagtaataacaaaaaagagtgggaatgcttctaggtctctcttcaattttataacttatttaagatctgggtaaggtctagctgattccatagtcaaattttatttgttcaataagcatccaacaaatattcattgaacaccagttatattctaattactatgaagctttttatggttaaaagagaaaagtttatccagacatttatagttgacaaagaactttagattatcaaatttgatctttccaatgtccctgtgaggtaagtagccccattttacaactaagaatgtagcggtaaatttcattttatggggaaaatgaggctaggagggttaaataacttgccttcagttaatggttctcaagcctgatggtgtatcaaaattccttagagagtcttaagccccattcccagagacttcgattccatgtacaaagggatggccccaaaatctgaatttctgatttcctcctctataggaggaataataattctaacaacttcacagaatggttctgaagtttactcaataagatatatgtaagccaggtgtggtggcacacatctgtaatcccagagactcagaagcagaggtaggaggattgttcaaggccaggagttcaagacctgtctctaatcctagcactttgggaggccagagtgggaggatcacttgtggtcaggagtttaagaccagcctggacaacatagtgaaacctccttctctaccaaaaaaaaaaaaaaaaattagccaggtatggtggtgtgcacctgtaattctagcgactcaggaggctgaggtgagagaattgcttgagcccaggagtttgaggaggcagtgagccatgattgcgccagtgcactccagcctgggtgacagagcaagactccaactcttaagaaaaaaaaaagaccgggagcggtggctcacgtctgtaatcccagcactttgggaggccgaggtgggcagatcacgaggtcaggagatcaagaccatactgtaaatggtgaaactccgtctctactaaaaatacaaaaaattagccgggcgtggtggcaggcgcctgtagtcccagctactcgggaggctgaggcgggagaatggcgtgaacccaggagaaggagcttgcagtgagccaagatcgcgccactgcactccagcctgggagacagggcgagactccgtctcaaaaaaaaaaaaaaagaaaaaaaaatacatggaatgtatgtaatcttagtagccatgtatttgagtaagccctgagttgcatagctcattaacattagtcaggattacacacagttcttctgattccaaatccctttctattacattaatcttcatgctgctgacagtaaagtatcactatcatagttattatattgtaaagactgctgctagagacttcatttgtacataaattatcccaaccagacaaaatgtaatttcggggctgggcacagtggctcatgcctgtaatcccagcactttgggaggctgaggcgggcggaacacttgaggtcaggagtttgagaccagcctggccaacatgttgaaaccccatctctaataaaaatacaaaaattagccaggcatggtggtgggcgcctgtaatcccagctacttgggaggctgaggcacaagaattgcttgaacctgggaggcagaggttgcagtgagccgggattgtgccatcgcactccagcctgggcgatagagcgagactcattctcaaaaaaaaaaaaaaaaaaaaaacaaaagcaaagaaaatgtaatttagggcaggcacaggggctcatgcttataattccagcactttgggaggccgaggcaggaggatcacttgaggtcaggagttcgagccaacatggcaagaccctgtctctactaaaaatgcaaaaattagccaggtgtggtggtgtgcacctgtaatcccagctactcatgaggcttgaacccaagaggcagaggttgcagtgagctgagattgcaccactgtactccagtctgggtgacagagtgagactgtttcaaaacacaaacaaacaaaaactcaatttaattcaggcctgccgcatgctaacaagcttttggagtacaattgtctcatctgttaaatgaagaaaattatggtcaaaagagaagatggaatttgtgagaaatacctaataaaagtgtctatcgcacttggcagctcaaaacagtcaaataaatttacctatcacccactacactgttccctttacaatctaaaaactaatctaaaagggaaaaaatctaattaaacaattatgtttacctgaaataatcgtgcaatggagagtagcattaatccaaataaaaagccattgtactgaaaatgaatatctggacttaggtcaaggaaagacaacagaggacaccaaattcataatgaactataccagctgatggagtatagaaattccaaatgctttaatagcaaaattaaagacaaaaaatactactactaatgtggactgctcataaatatgttttctataaacagattcaaattttttttttaattttttttttttttttttgagatggagtctcactctgtcacccaggctggagtgcaatggcacagtcttggctcactgcagcctctgcctcctgggtccaagcgattctcctgcttcagcctcccgagtagctgggactacaggcacataccaccacacccagctaatttttttgtacttttagtagagacggggtttcactatgttggccaggctggtttcgaactcctgacctcgtgattccccaaccttggcttcccaaagtgctgggattacaggcgtcagccactgcacccggcctaaacacatatttttatttttgctaatctctgtatcattccaattttgctgaagcaaacaccatagtcagatctttaattgaaacttacatttgggggtgtggtggctcacacctgtaatcccagcattttgggaggctgacatgggcagatcacttgggaccaggcgttcaagatcagcctggttaacatggcaaacccccgtctctaccgaaaatacaaaacttagtcagatgtggtggcacacacctgtagtcccagctactcgggaggctgaagcatgagaatcacctgaacccaggaggcggacactgcagtgagctgagattgtgccactgcactccagtctgggtgacagagcaagaatgtctcaaattaaaaaaaaaagaaaaggaaagaaacttatgtttaatgacatcttagactagatgatttaaaatatataaattttttttatttaaaaacaaatttatgtttaagaagaaaaacaaaactttcccaggtttttcctaccttttctaccttaaaggccccgcagctcaggggcaagaagttttttgttttttagaattagggatgagaaaggaatggacctaactgctactaaggccaccagcatgcagcattgcattgtttataggacatatacaatcttatgtattaagcctcacaatgaacctgtgaggtatttagaattttcctctttagagatactttagagattaagaaattaagcctcagagacattcatctactcatggttctaaagctgctaatggcaactaagatttgggtccaggttacattgaaaccagaggctgggtgctttcctccacaccaggctacctttatgaaaactgcaactattagtttccattacaaggattttaattacatccagaagccttgaatttaaaaactttgccttttaatatgtggtaagccaagatggtagaaaagcacaaatgcctgaccattcccacacgcacactgtgatatgccttttccaattaaaggtatttggaataatgtgttacaaaactatttaatatatatgtatcagattaatctaaaaatagttcacaatctcagtacattgttttattgatgatttagctggttctcacatatcccactaatttttcagaaatgaaatactctaacctcagagtccagtgaagttgggcagagaccacagttaagtgagcctactaacccaccccacactcattttcagaaggagctcacatctccaatcattattatagaaaacattaatcttgtattagtatacatcagaattagtaagggaatagtacatagaatatcaaggatacggtccacaattaataacccgaagttccacagaagtaataccgacagaataaattttggcttttctgtaagttctttacccactttttttccatcaatgcatttacagcacctacattgaaacattaggaaagaaacagaaacaactttgatcttcatcggaacatcacttctgcagagtaaactatgatacattagtcattcctgacaaactacatgccattcttttttttttttttttccaatttttctttttctttttttagaagggatggggtatcactgttgcccaggctgatctcgaactcctgggctcaagcaatcctcctgccctggcaccccaaagtccatgccattctttatttcttaaaaaggtcacagcaagccgagcagggtggcaggtgcctggctactcaggaggctgaagtgagaggaccacttgagcttggggtctgaggccagcctgggcgacatagtaagaccttctcactagaaaaaaaaaaaaaaagaaaaaatggtagcacaaacaaggacctcggggcctgttccacgtctttttctaagctgtgctgatctaaaacaaagtcttcatttaacaaaattaaaaataaaaagccacgtaaaccccaaagtaaggattttgaaaactgagagagcatacattatttcagtccttaagagtaaaaactgcagaccaggctaatttaacactctaaattatatatcataataccagccaaattaagaacagaactgcaaatgtgcacctcctgactgtccaaattataatttaaaaacctttatgtaaaaatgtatgtcatttaaatgttgagatatattactaagtgacaataacaggtgacaaaacagtaagatcctatttttattttatgaaaattatattcgaacacagaaaagagactgaactaacatacaccaaaatgttaatagtgattagtatacgtgggggagtatgactcatttttactttgcatgtgtgtgtgcttttccaaagtttttacaatggggatgtattattctataaaaagacgatattgaaaatgtgctacatttgaagagtggcaaattttaacaataaatctaaaacaaataatagatgcaattaaagggggactagcaaaggtatcctcctttcgccctatggagcctcagttccctcatgaatagaggcagtaaacttacagggtcactgagaaaattaaataagttaataatctgtatatcatgcttggttctgggaactgacacagagatttcaattttttttttttttttttgagacagggtctcagtctgtcgcctaggatggagtgcagtggcgtaatcacagctcactgcagccttgaccacccaggttcaagagatcctctgcctcaggctcccaagtagctgggacaacagtccccggcaaccacagctggctaatttttgtatttttagtagaggcagggtttcaccatgttatccaggctggtctcaaactcctgagctcaagcaatacactagccttggcctcccaaagtgctgggattaaaggcatgagacactatgcctggcctttcaacaatctttttaactattccctctactttgcagatggaaatgaattctatttcctctccctttgtgcttgtatactcttcccctggaagcttgaattcaaacattttgagttaaaaagagtatattaaaaattaccttccctttcaactcttgccaggtcataaacagattgaaaggaagggtttcaaacaggattattcaatgagaatgcgcaagggttaaagacattcccaagggactctttactgagtggggcaagggttcagtgtagagggcctcacaaacagaaacgatccatttatgacaagggtcactgaatgaacaccagtcactgttctgtcacaatcacaccagcgagcttaagagttaggaaagaatgaggtcggtgcagcggctcatgcctgtaatcccagcactttgggaggccgaggcgggtggatcacgaggtcaggagttcaagaccagcctggccaacatggtgaaaccctgtctctactaaaaatacaaaaattagcctgccatggtggtgtgcacctgtaatccaagctacttgggaggctgaggcaggagaactgcttgaacccgagaggcagttgcagtgagccgagatcgcgccacaatactctagcctgggcgacagagcaagattccatctcagggaaaaaaaagaaaaaaaaaaaaaaaaaaaaagaaaaaaaagaattaggaaagaatggaaactgacattaactgaaacttagtacttggaagtcactgtgcaagacattctatatatgtgtattcaatacttaaaattaacttataagtaaaagttgtaaaacataaaaatttaaaatataagcataacttaaaataacaaagagtcttcatgaaggaaagtatatggaaacaactaaaagcactcagtgaaatttcttgagtttactagaatgaaagctccgtaaaagcagactttggtggttagcacaatccatacgtgtgcgctactgtgcatgtactgtgtgcataccctaagatagggtaggtgatggaagcctctggcggcctgctgcaaggaagcaaagaaaaacatatggagctattctgggtgggagaaatggccctagaactatacttgggactacttcttttcacaacattcataatctcttgttctacctctgccttgtaactccaccttatccccatttcttccacaagattcagctagagtgctttaaacatgtagtttaaagcacaatacaatttttcattgttttagcatttgctatcatataaactttgttcctcctaaattgggagtaagttaatactacttaacactgtactattttttcaataccttccatacaaaatgacatgctccagacttactcacggacagcatacacaaagagtacatccataaagatgacggaaaatctctggaaaagtaaggtccttgagctggagtaattcaaattatggacattcagcatttcttgatcaaaatatttggcaacatgtgacaggatatactcaaaccatgcaaagaaaggggggtaatccaacgtccactctgaagttgcctgtgataaaaatagaagatcagacatatcctaaataactgaataaacaaagatggtgcaacgatttaaaatttttcattctctggtctttaaaacagcacaataaggctgggcgtggtagctcacacctgtaaccccagcactttgggaggacaaggcaggcagatcgcttgagcccaggagttcaagaccagtctgggcaacatggtaaaaccctgtctctatttttaattaaaaacaaacgaacaaaaatcacagtgaaatgctgttgcacttaaaatatgccctaaattaatagaacaattcctatgtaagactgaacagaacactaaaacctgtcttgtaaattatttaacatggtcttcaatttccttaaatagagtcaactacacttaaaggaaattaatttgaaacctggagatgataaacttggttatgacatggttctactcatctaaataacaaataattcactaacttaggttcaaattctgtcttagagacacaatttccctatttgtaaaatgaggtattatgaagattaaatgagataatacatatgaaatatatttataaacaacaaaataatacacaaatgctattatattttgtttcagtaagttatatgatcctggagaatagaaactatcctttatcattttgtatcctcagcatttagtaagtgcctgacatattgcttgggataaaataaccactagttaaaggaacagttgctaatgagtaaatgaaaacagaaatcatatgaaaatatacaagctttagaatagtatattttgttagatttctctctcagttttctttttctttttttttttgagatggagtcttgctctgtcacccaggctggagtgcagtggcgcgatcttggctcactgtaagctccgcctcccgggttcacaccattctcctgcctcagcctcccgagtagctgggactacaggagcccgccaccacacccggctaattttttgtatttttagtagagacggggtttcaccatgttagccaggacggtctcgatctcctgacttcgtgatccgcccacttcggcctcccaaagtgctgggattacaggcgtgagccaccgcgcccagcctaagtctcactttttgcacctatacaacagggatagtagtatctacctcatgggttatggtattccattttatatatacatataaaatactatatatggtatcgaaactcataggaaacttgcaaaaaatttgccttaaaaaaaactccctgggccgggcatagtggctcacacctgtaatcccagcactttggaaggccgaggcaggtggatcacctgaggtcaggagttcaaaaccagcctgaccaacgtggtgaaaccctgtctctactaaaaatacaaaaaaaaaaaaaaaatttcctgggcatggtagtgggtgcctgtaatcccagctactcaggaggctaaggcaggagaatcgcttactcaggaggctaaaggcaggagaatcacttgaacccaggaggcagaggtcgccagtcagccgagatcgtgccattgcactccggcctgggtgagagtgagacttcgtctcaaaagaaaagaaaacaaaacaaaatccaactccctgtcagcagagtactgcttttcatcaagaaatttctgggccgggcgcagtggctcacgcctgtaatcccagcactttgggaggccgaggcgggcagatcacaaggtcaggagatcgagaccatcctggctaatatggtgaaaccccgtctctactaaaaatacaaaaaattagcctggcatggtggcgggcgcctgtagtcccagctactcaggaggctgaggcaggagaatggcgtgaacctgggaggcggagcttgcagtgagcagagatcgcgccactgcactccagcctgggcaacagagcgagactccgtctcaaaaaaaaaagaagaaatttctggagtaatgtctgtttttcataaagtattacacacagtttagtaattcataccagaaacaaaaatacaagctgggtgcagtggctcacgcctgtaatcccagcagtttgggaggccaaggtgggtggatcacctgaggtccggagttcgagaccagcctggccaacatggggaaaccccctctttactaaaaatacaaaaattaactggggcatggtggcgggcgcctgtaatcccagctacttgggaggccgaggcaggagaatcgcttgaacccgggaggcggaggttgcagtgagctgagattgtgccactgcactccagcctgggtgacaagagcgagactctgtctcaaaaaaaaaaaaaaggaaaagaaaaagaaataaaaatacagacatagtttttataaacaatatgagagaatggtttgtatattacctgatgtctacacatcacatttgattattcctttcctcacaaaatcacaaaacattaggaacagacaggatcttagaaataattcactccaaatcctcatgtgccagatgagacactaaggcccagagacatgaagtatcttttctctatgtaactagatagtggcagagccaggcctaagaatccagttcttcctctttcccagtccactattctttcaaaaagtgtggtagatattatacctcccaatttatagactgggaaactcattttgaagtaaggggattccctagtagtgcaagaggacagaactgagaagagaccacaatttttataaagattaattagatctatgattgcaagaaaatttttttttttttgagacagagtctcactctgtcacccaggctggagtgcagtggcgcgatctcggctcactgcaacctccgcctcccaggttcaagtgattttcctgcctcagctcctgagtagctgggattacaggtgcccaccaccacacccagctaatttttgtatttttagtagagacagggtttcactgtgttggccaggctggtctccaacccctgacctcatgatctgcccgccttggcctcccaaagtgctgggattacaagcgtgagccaccgcacccagccagaaaacatttttaatatcagtaatatttacctagtttatagatgaatcttaaaaaaaaaaaggtgaaaattaaaacttacctcataataccactgtgatattggcaaactgtgagtgatagcaagccagtttcggtgtacttcaaaatctgtggaatggctactcaaaacaattagataaataaaatgagattttgcaatatttatcaatttctaattcccatagttatgcttttaaatggactgacattccctaagctgtcacagctcactgtcctatagagacacaaatagacattacataatcctgtaaaacctcatttatctggaggtcagatcttctagaaagttgttgagcaatctacaagtcagtgaaaagggtctcagaactacctaaagggctgacaagaagcccagactttctctttttcttttctttttttttttttttgaggcggagtctccttctgtcacccaggctggagtgtagtggacccatctcagctcactgcaactgccgccccccgggttcacgcgagtctcctgtctcagcctcccgagcagctgagactacaggcatgtaccaccaatcctggctaatttttagtagaggtggggtttcgccatgttggccaggctggtctcaaactcctgacctcaggtgatccacctgcctcagtctcccaaagtgctgggattacaggcgtgagccaccacgcccggcctagcccaaactttttcttacaggcggccctcaggcctttgctcagaagtgacttcctcttgctcacaaacaattctaacaaaattcattacctgaaaaaggcctcatataccaagacataaggatgaggagttcatctgacagagaaggtgggaaaagatccttgctttgcctttcccatgtctccaaattctccctcttcccatcactatacatatctaaggatcacctatctttagaaaaaatcttttcctgtttgtccactgctgtacctcccctccacatttagaacagtacctgacgcatggacaagctcaatacattcttgctgaatatccactcaccaaagtgaacaatgagttccacagcaaagcccaatttaccctttccaacattttacacaacctttccacaatgacaactttctcctacctccagccaacccttctttctcagtcatggctttcccttcctatattcatcagggacatgttgatattcccctaggttcagttcttggtcccctttttacgcaacttattctgctctggacaatcttatgcaaactttctggttccactgtcaattgtataatgatgactcccaaattacttttttttttttttttggagacggagtctcgctctgttgccaggctggagtgcagtagtgcgatcttggctcactgcaaactccgtctcccaggttcaaacgactcccctgccccagccacccaagtagctgggactacaggtgcgcggcaccacgcctggctaatttttttttttgtattttagtagagatggggttttactgtgttggccaggatggtcttgatctcctgacctcgtgatcagcctgcctcggcctcccaaagtgctgggattacaggcatgagccaccatgcctggcctactcccaaatttcaatatgcaacattggcttctttactaagttgtggagacagacacctccaacttcctgttaacaatctcacttgaatttcctacagatatttcaaaatctccactcagaactgaattgatgacccatccatcccaagtctaagtacgtatacccaacagaaatgcatagtatgctcactaaaagacacgtactgtaagaatgtgcaaagtggggccgggcgcggtggctcacacctgtaattccagcactttgggaggctgaggcaggcggatcacgaggtcaggagatcaagaccatcctggctaacatggtgaaaccccatctctagtaaaaatataaaaaattagctgggtgtggtggtgggcgcctgtagtcccagctactcaggaggttgaggcaggagaatggcatgaacccaggaggcggagcttgcagtgagctgagatcgcgccactgcactccagcctgggcgacagagcaagactccgtctcaaaaaaaaaaaaaaaatgtgcaaagtaaaactatttgcaacaggtaaaaagtggagataaatgtccacaaacagcagaatagctaaataaattgtggtgttttcacacaatggaatactgtagagcagcagttctcaaagtgtgattcagggactcacagggagaccctttcaaggagtttgaaatatgtactattttcataataatattcagatgtaatttgttctactttcactcattctctcacaagtatacagtgatgttctagaggctacataatgtgatgtcatcactctgaaggccagtgaaatttgtgcttgtatattcttgtgcttgaacaatttgttttaatttctaaaatggtaattatctattatacagcccacataaacatatagctctttgtgaccctcaattatttttaagaagtgattctgactaaaaagtttgagaaccatgctatctagtgataagaatgaaaaaacagtaattacacaacaacgcacaaacttaatgttgagtgaaagccagacccaagagaatagattgtatgattacatttgtataaaattcaaaaacaaacaaaaaaaacccaaacaaaaattcaaaaacaggcaaaatttaacctatgcggttaaaagaacatcagaggccaggcatgatggctcacgcctgtaatcccaacacttcgggaggccaaggcgggtggatcacttgagctcaggagttcaagaccagcctgggtaacaatgagaccccatctctcaaaaaatttaaaaattagccaggcatggcggcacgcccctgtagtcccagctactcaagaggttgaggtgggaggactgcctgagcccaagaggctgaggctacagtgagccaagtttgcaccactgcactccagcatgggagacagagcaagacccggtctcaaaaaaaaaaaaaaaaaaaggtgagaatatcaatacctttagtggggtggggttgtggggagtggctggaaagatgcccgagggataatattctgcttattgatctatatgctttttacagatgtattcactttgcaaaaattcagtgagctgtacatttatgatttgcacttctggtattttatactggattttgtatggaattctaggttgaaatttacttccactgaactttggaagcttattccattggtttttagtataaaatgttgatgttggtaagtctctttactttatggttttcccttcatctctggaagtttcctgtttctcctttttatccatagcagtctgaaatgtctagtgattaatgtcttcatgagggtcttttttcattaattatgctgagtatgtagtgccttcagctctgggaaatttattatttgttaatctcctcccctgtgttttttttctgaaatttttttagatggatgctagacaccctagattgaacttataatttttatcttttctcttatatttttgaaagatttactcaatttgtccttaattttggcaataacattttctattaaccactgtttctctgataattcctgtttcatagcatctgttcttgttttatagacatgtcttttttactctctttggagacagcatttacaaaatctgagtgagtttggggacactttcttttcctatatagttgtactatatagactttttccagtctcttttttgggtgctttattttgttgtttcctattcaaaactgttggagacttcacttttgttggagtgctgggattacaggcgtgagccactgcacccatcagtaaaagtaatcttaattgctgaaatgacacttgaaaaaaaaagcaaaactaagactagagacataatgataaatagtaactacttttttaaattgcattaacaaaatctacaatcagccgagcacaatggttcatgcctataatcccagtactttgggaggccatggtgggaggatagcttgaggttggaagttcaagaccagcctgggcaacataatgagactcccatctctacaaaaaacttaaaaattagctggtgtggtggtgtgcacctgtagtcccagctactcaggaggctgaggccagaggacggcttgagcccaggaatttgaggctgcagtgagctatgactgtgccactgtaatctagcctgagcaacagagttagactctgtctctaaaatttttgaccaccatctccagctaattctttgtattttttgtagagacggggtttcattatgttgcccaggctggtcccaaactcccaagctcaagcaatctgcctgccttggccttccaaagtgctgggattacaggtgtgagccactgcacccagccctgtctctaaaaattaaaacaaacaaaaacctaccatctcttacttgtcctatgatagcttcttaaattgaactccccatcttcactcaggcccttcattaatctagctttcacattgcagccaaagtagtatttctaaatcacgaatgttatatttcccttttaactggcctctgccctctggataaaatctaaagtactcaacatagtctaaaagatatttttggatctcgcttactcaccttttcagcccatttctcaccatcctcccacctgtctttgtcttactctacattcaccttttccctctgcctgcaacactctgtgtgaggtaggttctttaactcttaccatttcaccagtggcgtagagaagcagcttgccctgtcatgtagccagtatgcagcagagccagattctaaccagggcagtctgactccctagctacggctcttaaaccaccaaatacttccaaagttagttgttattttctctgcaaatccttcctgacctcaaggcaaaataaatctttaatttgtccatttctatctctaatgccaccactccaattcaagtcagcatcttctctcatgtagactttctcaaagggcacctagccttcctgactctccttctgcctcctccaatcatttatatgggagccaaaaaatattttataaatgcagatcatttccactgagcttataaaatcaaaatctaaatttttcatcctggctttataagctccggcatgatttggtgcctatctttccaatttcactgtgcatgtttctgtctctgttgcttattccatgccagctacactggccttatcatagctgcccaaataggtcaatactttcacaccacagggcctatatttatacagtgtgctctttcagataagttctacacacttgcacacaccacatactctgcatggatggccctctcattcacactttaggcctccaatgatgtcagttcctcagagacattctctgacacctaactgaaagtacatcttccttcttggtcgcctgataggatcctgttcttcttcttccattttttttttttttttttttttagatggagtctcactctgtcgaccaggttggagtgcagtggcgctatctcggctcactgcaagctccgccttccgggttcatgccattctcctgcctcagcctccccagtagctgggactacacgcgcccaccaccacacccggctaatttttgtattttcagtagagacggggtttcatcgtgttaaccaggatggtctcgatctcctgacctcgtgatccgcccgcctcggcctcccaaagtgctgggattacaggcgtgagccaccacacccagcctcttcttcctaatacttaatccaaattgtagttatgtaactatatatttgcattgatttgtttcttgtactgttatacttactgaactacacacttcacagggctagggatcatatctattttttcctcccttctagcctagcaacagtacctggtacataataaagcaattagtaaatgttttgttgaatatatctgcatttatcctgtaacttccctctcatatcccaggggtttctggtttgagaaacagagcattaactacaattaaaaacattcgtttttaaatatatgaagtgtttcataaatttgcatgctatccttgtgcgtggaccatgctaagcttcctgtatcattcgattttagtgcaggcataccttagagatattgtgggtttagaccaccataatgaagtgaatatcgcaataaagtgagtcacatgaattttttgatttcccagagcatgtaaaagttatgtttacactggccggccgcggtggctcacgcctgtaatcccagcactttggaggccgaggcgggcggatcacgaggtcaggagatcgagaccaccctggctaacatggtgaaaccccatctctactaaaaatacaaaaaaaattagccgggcttggtgaggggcgcctgtagggtcagctactcggtaggctgaggcagaagaatggtgtgaacccggaaggtgagcttgcagtgagccgagatagcgccactgccctccagcctgggcgacagagcgagactccgtctcaaaaaaaaaaaaaagttatgtttacactatactataaaacgtgcaatactactatgtctaaaaagacagcgtacaccttcattttaaaatactttatcgctaaaacaaagctaatgatcatctgcaccttcggcaagtcctaatctttttgctggtggaggattttgcttcaatgttgatggctgttgattgatcaggtttatgggtgctgaagattggggtgacttgtggcaaactggtttttttttttttttgagatggagtctcactcttgtcacccaggctggagtgcaatggtgtgatcttggctcactacaaccttcgcctcctgggttcaagtgattctcctgcctcagcctcctaagtggctgggatcacaggtgcccaccaccacacttggctaatttttgtatatttagtagagacggggtttccccatgttggccaggcgggtcttgaactcctgacctcaggtgacccgcccgcctcagcctcccaaaacgctgggattacaggcatgagccactgcgcctggcaagactgtggcaattgcttaaaataaaacaacaatgaagcttgccacattgatgggctcttcctttcacaaaagattcctctagtatgcaatgctgtttgataggattttacccacagtaaaactttcaaaactagagtcaatccttcttctcaaaccctgccactgattattaactaagtttatataatattctaaatcctttgttgtcatttcaacaatattcacagcatcttcattagcagagtgcatctcaaaagaaaccactttctctgctcatccatgagaagtaactcctcatccactcaagttgtatcatgagatatcatgcagcaattcaatcacatgctcaggctccacttctagttctcttgctattcccatgacatctgcagttactttttccaatgaagtcttgaacccctcaaagttatccattggagttggaatctaattcttccaaactcttgttaatgttgatatcttgacctcctcccatgaatcatgaatgttcttaatagcatctagaatggtttcaatttactttgcccagatccatcagaagaatcactatctatggcagatatagccttatgaaatgtatttcttaaataacaagacttaaaagtcaaaactgattgggcatggtggctcacgcctataatcctagcactttgggaggccaaggcaggcagattgcttgagcacaggagttcaaaaacagcctgggcaacacagcaaaaacccatctctactaaaaatacaaaaaattagcagggtatggtagcacacaccttaagtcccagctacttggcaggttgaggtaggaggattgcttgagcctgaaaggtggaggttgcagtgagatgagatcatgctactgcactacagcctgagaggcagagtgagaccctgtttcaaaaaaaaaaaaaaatcaactactcaactggctgcagaatagatgttacatcagcaggcatgaaaacaacattaaggttgggcacaggggctcatgtctgtaatcccagaactttgggaggcaaaggtaggaggattgcttgagcctaggaattccaggccggcctgagcaacacagtgaccccatctctccaaaaatacatttaaaaaaaaagccgggcatggtgctcacgcctgtaatcccaacactttgggaggccaaagagagcagatcacttgaggccaggagttcaagaccagcctggccaatgtggcaaaaccccatctctactaaaaatataaaaattagcagggcatgatggtgtgcacctgtaatcccagctacttgggtggctgaagcaggagaatcactcgaacccgggaagctgaggttgcagtgagctgagattgtgccactacactccagcatgggagacagagtgaaactccgtctcaaacataaataaataagaaaaaaaaaaaagaaaaataatgggtgggtgcagtggctcatgtctgtaatcctagcactttgggaggccaagacaggtggatcgcttgaggtcaggaatttgagacgagcctgggcaacatggtgaaaccccatctctgcaaaaaatacaaaaagttagctgggtgtagtggtgtgcacctgtagttcaagctacttggggagctgagatgggaggattgtttgagcccaggaagtagaggcagcagtgagctgagatcataccagtgcactccagcctgacaaagtgaaacactgtctcaaaaaagaaaagaaaaaggaaaaatattgaccagtgaggtggctcaggcctgtaatcccagcactttggaagggctcacttgagcccaggagttcgagatcagcctgggcaacacagtgacacctcgtctctaccaaaaagaaaaaaaaaaaacaaaccaaaacaaaacacacacatatagatagatagacagacagataatagagagagaaaggaaaaaaaattagctgggcatggtggcatctgtctttgtcccagctattcgggaggctgagggaggaaggctgctcaagcgcaggagattaaggctgcagtgagccatgatggtgccgctgtacttcaccctgggagatagagcgagaccctgcctctagaaaaaaaaaatgtttctgagcagtaggtctcaacagtgggcttattcagtaaaccatgctgtaaatagatgtgctgtcatccagggctttgttgttccatttatagatcacaggcagagtagatttagcataagtcttaagggccattggatttttagaatagtaagtgagaaatggctcaactagatgtcatagatggcatcttccaatgtaagtctgtttcttctaccttgaaaacctattgtttagtgtagccaccttcatcagtgagcttagctagatcttctagaaaacttactgcagcttctacatcagcacttgctgcttcaccttgtactttattatggagatggcttcttttcttcctttttttttttttgagacagagtttcactcttgttgcccaggctggagtgcaatagcgcgatctcagctcactgcaacctccacctcccgggttcaagtgattctcctgcctcagcctcccaagtacctgggatcacaggcatgctccaccacgcccagctaattttgtatttttagtagagacgagatttctccatgttggtcctcaggtgatccgcccgtcttggcctaccgaagtgctgggatcacaggcatgagccaccgcgtccggccgatggcttcttttcttaaaagaacctctgctagcttccaaattttcttctgcaacttcctcacctctcttaggcttcaaagaattgaagacttagggccttgctctggataaggttttggcttaagggaatgttatggctggtttgatctatccagaccactaaaacattctccatatcatcaaggttgtttcactttcttatcacttgtgtattcactggagtagcacttttagtttccttcaagaacttttcctctgcactcacaacttggctgtttggcacaatgggcctagctttcagcccgtcttggctttcaggatgccttcctcatcaagctcaatcatttctagcttttgatttaaagtcagagatatgtgactcttcctctcacttgaacacttagatgccactgtagggctattaattggcctaactttattattgtatctcagtcaacagcaaggcctgaggagagggagagagatgatggaacagctggttgatagagcagtcagaacacacagcatttattaagttcaccatagtgttgccccaaagcaatcagaatagtagtatcaaagatcactgattacagatcataacaaatataatagcaatgaaaaagtgtgaaatatttcaagatttaccaaaatgtaacacagacacaaaatgagcacatgatgttagaaaaatggcattgatagagttgctcaatggagagttaccgcagccttcaatttgtaaaaaaaatgaaaatgcagtatctgtgaagtgcaataaagcaaagtacaaaaaatgaggtattcctgtacatgtgctgccaaagtgagcgctatatacattcttaacctggaccaagagcagtattcaaaataacaactctggccaggcacggtggctcatgcctgtaatcccaacattatgggaggccgaggcaggtggatcacttgaggtcaggagatttgagatcagccctccgccatcatggtgaaaccccatctctactaaaaatacaaaaattagcctggcgcggtggtgcaagcctgtaatcccagctactccggaggctgaggcaggagaatcgtttgaacccgggaggcagaggttgcagtaagctgagatcgtgccactgtactccagcctgggtgacagagtgggacgagactgtctcaaaaaaaaaaaaaataacaaacaaaaaaaacaactctgaggatacatttctagcctgaggcaactaaagcaagaaataatcttatgcacagacctacagttaatcattttactgaatctacatatacattccacttacatgattacatatacgcatcagtatggacttacattctatacatatgactctcccatattcaacaagagatcaccaagagcctgaatcaaaggataccaataaaacaaaccctaaactaggaagtgggagggaaaggagaaaagggatgagggagaaaaagaaaaaagaggaaggatgaagggaaggcggaagccaatattaataaatactgagtacctactgtatacacaccaccatttcatggttgcttcttggtttccacgctttcagtcttggccctacatccattttttacacagcagtctaactgtggtcttcgaaaccctgtgttatctgtccccagtgcaccccatgctgtgccactcctctccctcaatgctctgcagccacaaggggcactttctgttcctagaatgctctgacttctttcctgccacagggcctttgaacttactctttcctatgcctgaactgctttccacatcacttattctttttcattttttgaatttcaactcaaacatcaccttctaaaggaatagcgcacagccacttagtaaccctctatttcctttatcacaatctgaaaagttcctgtttgtctagctccacccaccaacttcgagggcaaggaccagcaatgtcttgctcaccaccgcatctcccgggagttagaacggtacctggcacctcaggcatctgataatactggctgaatggctgttggaggcagaggttcttaactgcctattaaggatgaggaccctgaggttcagaaacgttaaagtgatttgttccaaatcagacagcgccaggtctgaacctagccagctggggctaagtcaagtaacaactggcgaaacagaaagcttagcaaaggcaggatagcgacaaacacgacctaaagttttctcttcatacccagggatatccacacctttctctcccgccctgaccgaccgcggggcctccccgcccagcccctggccgtgcgagtcccttactatgtggggatgagaaggcatttgagaagagtcaccccgagcgccaaagccgaaaaccaattgccagtacccgtggcaattgtgagcgccgccattgctgcggcaccgcacgcttcccaccaacttgatccacatccgggatcccgcgcatgcggagaaagccctctgaagccgtgcccgctagctgcgcgcatgcggcgagcggcgcagccagtccggggactgcagtcagctatttaaacctcccgcccaccttttctttagacccgcgtctcaccccgggccggaagggctcctgcgcaggcgtttgtagccacttttaagttttatcagctagttcatgcttgcgttgaaagagtggtcgtttgcgctgggtcatcactgtgtagtattggggatacttaggtgagaaaaaaacttaacgctagagacgttcacgcactagtggagaagccaggattgttgccctagagttacagtagataaaagtacctcagagaactgcgggggctcccaacctggacgcttgcaccggagtattaaatccagctagagaatggcatgtgcaaagatacagaggtgagaaacattgtgtttttagaactctgagcgaggctcttggctcacctcctgcttgagcggaacccattctggaagcagggtagaggctagtcctaacgcttagtgtacaaatagcctacggttcatgttaaaataattcggattctgattcagtaggcccaacaaactcgcagattgcgtaatgactgaggcacatgcaatttaattaggcttgtctcattattttactgtatgatcttggacaaactaaactctaaacctcagtttctttttttaaaatttttatttatttatttatttttgaaaataggagataaaaatggtttctggccggacgcggtggctcacgcctgtaatcccaacactgggaggccgaggtgggcgaatcacctgagttcaggagtttgagatcagcctggccaatatggtgaaaccctgtctctactaaaaattaaaaaaaaattagccgtgcatggtggcgggcgcttgtaattccacctactcgggaggctgaagctagagaatcgcgtgaacgtgggaggcggaggttgcagtgagctgagatcgcgccattgcacttcagcctgggcaagcctcaggcctgtttatccaagatgcttttggtctaaaaaaaaaaaagtttcagcctcataaactggttgtgaggattaaatgagaatgcatgtaaattgcttgatatgattccaggacagaataaatgctcaataatgtgtcagatcagttaattatcttgctgaaagatgcacagcaagtaggtgacagtgtcacctccctaatatgagtttatgccctcccacagcttcagctgccacccatgtgaatatgattcacagatttctagctcagcactcttctctcactttcagaccctggtctccagttggctacctgacctcacgtagatgtttttgttttgtttttgagagacagggtctcattctgtggcccaggctggagggcaatagtgcaaacacagctcactgcagcctcgatctcctgggctcaagcagtcctccccacctcagcctcctgagtaactgggaccacagggatgtggcaacgtgtgtgtgtgtgtgtgtagagatgggatctccctatattgtccatgctggtttcaaactcctgggctcaagtgatcttcctgcctcagcctcccaaagtattgggattacagttgtgcgccaccatgcccagctacttagatgttttaaaggcactttgcattaattttctatttctgctgtaagaaattaccataaacttagtagcttaaaacaacacaaatttattctcttacagttctgtaaaccagaaatctaaaatcagtcactggagtaaagtcaaggtgtcatttggattggttgcttccagaggctctgcagggagattgtttttcttgtctttttcagcttttagaagttgcctgcattccttggccccttcctcacatcattccaaccaacttccatccttacaatcgattactttcctgtaatagctacagtcaaatgggcagctgaagatgatgatgatgatgatcttgacaccgagaagcagaagaccaatgaagatgaccagacagcaaaaaaggataagttaaaagaaggtaaaagaaggccactcgtacttgctgtctcagtatctaaaggtggtcaccttccagtagagaggcccacccactgcagaccgtgccaccgaaggtgacatgcactttctgccacccatccaaaaccgtaacgtgaatctgcaacaggagaggaaagaagaaccaaaacttccaaggccttgcattttttcttaaaagcatttttttttttttttggagatggagtctcactctattgcctggagtgcagtggcgtaatttcggctcactgaagcctctgcttcccgggtttaagaaattctctgcctcagcctcccaagtagctgggattacaggcacatgccaccacacctgtctaatttttgtatttttaggagagacagggtttcaccgtgttggccaggctggtcttgaactcctgacctcgtgatccacctgccttggcctcccaaagtgctgggattacaggcctgagccactgcacctagccaaaagcattttttaaagaaaaattggtctatattttttgtttctactttatatctttgtacatatttttagagtgcaggcatttttaataatcttggatgaccaaagcagctttcagaatgttctttgcccaacttctgactttacttgtagtgtgaccatgttcattagaatctcaaaggaggaagaaaagaaaaaacaaacttgtaaaaaatgaaaaatagatctggcgaggtgattcacggctgtaagcccagcactttgagaggctaaggcgggtggatcacctgaggtcaggagttcgagaccagcctggccaacatggtgaaatcctatctctactaaatatacaaaaattagccaggtgtggtggtgggtgcctgtagtcccagctactcaggaggctgaggcaggagaatctggaaccccggaggcagaggttgcagtgagccgagatcgcaccattgcactccagcctgggcgacagagtgagactatctgaaaaaaagcaaaaataggccaggcatggtggctcatgccttgatcccagcactttggtaagcctaggcaggtggatcacttgaggtcaggggttcgagaccagcttagccaacatggtgaaaacctgtctctactgaaaatacaaaaattagccaggcgtggtggtgggtacctgtaatcccagctacgcgggaggctgaggcatgagaattgcttgaactcaggaggcggaggttgcagtgagcagcagtcatgccattgcactccagcctggtgacagagtaagactctgtctcaaaaataaataaatgcaaaaacaacaaatcttattttttcagcctgttcgatgtatgtgtgaaacaatgttgtccaacaataaacgggaatcttatttttctgaggttttttcccctaacttcatttctagtgaattttctgagttctaacaaaaaatggtagagacagttgtcatgaattgtaccccttttctcattctttctgctttaggcttgaacatgaggcataaagctagactcagctgttaggaaaaactgcctcatgctggccctgacatcattgagctaataaacaaatccctctacctccagacttattatgtgaggaaacacacacacacacacacacacacacacacacacacacacacgtgttactgaaacaccaggggttccgatctagttcctgttccttgcttcacaaaaagccaatcactgagatgattattgccaaagaataaggctttaatcaggtgctgcagagaaacagatgggagatcaatctcaaacttgggtccctgactgactaaaactaggtttatatagcaggaaagaaatgtaataatgtgttaaaaaaaaaaaaaaaaggaactagggaggaataaggaagcaatcatgatgaatgaggggcttggcatctcattttctggatgctgtgatccggtgagtttcagttctttgatactttttttgagagacctgaaggtcatttcctgaggaaggaactcagataaatataactttccagctttaagaccagaagggtcaatttctatgtttgtccaaaaaaaaactaaggtactattgggtcaggttcacacacgcccacaccacccttaggtgtttaagccacagttggtcagtttacctgttcttccagcaaaagcatttctcactgctcttgccaggtcctgtctatttaacctcctagatgtctctcacattcatgcattcctcttcacccacctttgccactctaggcataatgacctgctttcagcctttggtacatgccatgttccctcctgtcactgccctttatacacgcccttccctctgtctggaatgttcttccttctccccttaggctagttagctcttcatcctttggcttttatagcaagcactgcttccccaagacagccttccccaacctccccttatcctaccctgggcacctgtcagctggatgcaattttatactatgtgtgtgattatccaattatctcactagttattcactccatgaggacagggaccttttctgttttattcatcattgtagcagcagcaacccacatagcagctgacacaacagatacgctctatatttgttgaatggcatgaatgtctaaaataggattccaacccagtctctcctacctgagtctctgcttatcctcagtagctgggactacaggcatggcatgtgccaccatgcctcttcggttcacctttaaaaaataaaagaattggccacatacaatggctcacacctgtaatcccagcacttcgggaggccaaggcaggtggatcacaaggttaggagttcgagaccagcttggccaacatggcaaaaaccccgtctctactaaaaatacaaaaattagccagacgtggcaggcacctgtaatcccagctacttgggaggctgaggcgggagaattgcttgaaaccggaaggcagaggtttcagtgagccaagatcttgccactggactccagcctaggtgaaagagcaaaactctgtctcaaaaaaaaaaaagaagaatgaaaacaagtctctaggcaatcagaacccatagagcggctgcagtttgtgagatcatcaaaatctggacactccctttcaaagaaacgtgtagtggtcaggaaagctttccagtcaccggcaatggccaggaactggagtagagtggactgatttcatagaaagaaaagaggccaggtcacatggtttatgaaacacttcaacgatctgctcagagttttcagcttgaagacccctatgaaactgaccttggcccttttctgcaatgagtatatttgagacatggggagaaattgcagaaaggtgccgatgtcagttttatagggtcttcaagattatgattaattgaataagttatatgtttttagacttcgggatgattgtttataatgtcacttggagggtaatgaaactcagaaagaaacaggccaggtgtggtggctcatccctgtaatcccagcattttcggaggccgaggtgggaggatcgcttgagctcaggagttccacaccagtctggccaacatagcaaaaacttgtctctactaaaaataaaaataaaataaaaaaattagccaggcatgatgatgcatacctgtagttccagctactggggaggctgaggtgggaggattgtttaagcttgggagattgaggctgcagtgagctgtgattgtgcgccactgcattccagccttggcaagagagtgagaccctgtctcaaaaaaagaaagaaagaaacagtccagttccaagtttaaaattagaagtgaatagtattgcatttatatgccatcctagctatttattgctgcataacaagttacccaaaacttagcagcctagaaacatttattatttcagagtttctgtggtcaggaattcatgagtggcttagctaggtggttctggttcaggttgcagccaagatgacaccaggggctgtagtcaactgaaggctccactggagctgtagtatccacttccaaggccaacagaagccctcagttccttatcacatgatccaccttccttggcctcccaaagtgctgggattactggcatgagccacagccccaggccccaaatgcccttttcctttattgtatgtcaggctcctcctatcctatgacaaatcctgcatgacccagttcagatgatctatgaaattttcccaaagcacttcatgcaggtttcttttcagaatttacaattgtaaccccgatgacagctaagccctccaagattcttcaaatctaaataaaactgactttgcccaaccatttagtctcaatttgacagcctgtggaagccaaggctttcccttgtatttctgcagagtctcagaggcatacattttcacatcacctcttttctcctggacttcctaattctcagttgtggcctgtgttcttaccttataacccagattggagattcagctccttcactgctttccaggcaaaacctgctgatacacagtatttatgagtacagagactttactgacgagcacagcacacacagcaaatcctttaaatctcaaggcatcaggctggccctccctggtcctctttgactctttggccatgtggtgtccccctgttgccctttcctggtgcagtttcagtctccccatttttctgatcttccctcactctctgaaacaacaaacctcttcgaaagcctttcttgtgtagagtagttccagggcgctttctgttcttggcctctccttctacccaagaccttatgaaggtgttattccaaagaagacaattcctttcccaagaggagttcttggaccaacaggataataattttccagtcatcttgtatgtttataagcatgtcttagttcaggctgctataccgaagtaccacagactgagtgtcttataaacaacagacaaacagacatttattcctcacagttctggaggctggaagtctgagatccaggtccaggaagcgggtctggtgagggctgtcttccaggttgcagactgccaacttcttgtatcctcacatggtggaaagagagtgagagcgctttctgggctctctttcatttttatttacttatttatttatttttgagacagagactcactgtgtcacccaggctagaatgcagtggcatgatctctgctcactgcaacctctgccaccagggttcaagtgattctcctgctttaacctcccaagtagctgggattacaggtgccagccaccatgcctgggtcatttttgtatttttagtagagatggagtttcactgtgttggtcaggctggtcttgaacccctgacctcaagcaatccacctgcttcagcctcccaaagtgctgagattacaggtgtgagccaccgctcccagccagtgggctgtcttttataagggcactaattctactcatgagggctcaaccctcataacctaattactttagacaggcccccacctccctataccatcacattgaaggttaagatttcaacatatcaactggggtagggggctcaaacattcagtctataacagagtgtgcctcaccccatgggtctgaatcaggtctggtctgagttggttgtcccagatccctgcacagagcttggaatatagtaggccttcagtaacatttgttgaataaatgaatttgtgataaatgagaatgattgattaaaaaatactgaataaaataatgaatcagctaggcgtggtggctcacgcctataatcccagcactttgggaggccgaggcgggcggatcacctgaggtcaggagtttgagactagtctggccaacatggtaaaaccccaactctactaaaaatacaaaaaattagccaggcatggtggtgggtgcctgtaatctcagctacttggcaggctgaggcaggagaatcacttgaacccgggaagcagaggttgtagtgagctgagatcgcgccattgcactccagcctgggcaacaagagcgaaatttcgtgtcaaaaaaaaaaaaaaaagaatgaatcagttgacattaattgacctgatggttcccagggatctttgccctgcaaaagaagaagctgacagaagttctagtcctaattctagtcccagttttgcctctcagttgcgtgtaatcatgggcaggtcacttttcctctctgtacctcaggtgaaaaatgagaaggaggcttcttgttattcctttttgttctgccaaccacacctcattctaatcgtcacctgcttgccacctgatgcttcatgctgggacctggctggcctgctgggtagcttattattaaaatccacctgcctggttaaaaagtgctttcaaagcagagggcccacctggaattgttctagctcatgattcttgttcagccacaccattgcattccttgccttcctttaatgcctgttacaactgcacattgaataagctctttcattctcctcccaatttacgaatgaaatcccatcaatcttttcccatgcccagccctcacgcaggagtgtgtgtctatgtgtttgtgtgtgtgaatgcgtgtacctgcattcacttgcatgcatacacacacaccctcttccttaaccaggtttattgtgtctgaaattttccgaaactttgtataggctttctgaaggaagtagaaaccccatgtagtatttgtgttttttttaagacagggtctcactctgttgcccaggctggagtgcagtggcgtgatcttggctccccgtgtagtattcgtagcaaggctttggtcatagttgctttttcactgtttgaggccttcactggagctatctaccatgagtcctcagaggccaggatttccaaggagtaactgatcagttttcattcgttcttagctcattgatatcactaagcctctgtgtcaataactaatgtaagtatgtatgtatgtatgtaagtgcagtggtgtgatcatagctcactgcagcctcaaactcctgttctcaggtgatcctcccatctcagtctcctgagtagctgggaccacagttacgtgccaccacacctggctaattaaaaaaattttttttgtagagatgggtcttgccatgttgcccaggctggtcttgaactgccaacctcaagcaatcttccctccttggcctcccaaagtgctgggattacaagtgtgagccactatgcctagccaacatctaagatcttttttttttttttgagacagggtctcgctctgtatcccaggctggagttcagtggcacgatctccgctcgctgcaacctccgcctcctgggttcaagtgattctcctgcctcagcctcccaagtagctggattacaggtgcgtaccaccatgcctggctaattttttgtatttttagtagagatggggtttcaccattttggccaggctggtcttaaactcctgatctcaagtgatctgcccacctcggcctccaaaagttcttggattataggcgtgagccaccgcacctggcacaacaactaagatctttcttcccgcctccctgctcccccagagacagtctcgctctgtcaaccaggctggagtgcagtggcgcaatcttggctcactgcaacctccactttccaggttcaagtgatcctcccgtctcagcctcccaagtagctgggattacaggcgcgtgccaccatgcccagctaatttttgtatttttttagagacagggtttcaccatgttggctaggctggaaaactaagatcttaatttgagtgccaaagggtgtcccacagctcctttcctatattttgttattggggcggggccaggacacacatttcagtaatctttgcttttaagggttatgaggtaacttaaaatatggaaaaaaaaaaaacaggtataacacacagaaccttagatttcattgttgccaagctccatgtgaactaggatatgaatagtagcaatgattcaattattattaatagcatacacttactagataccaaacactgggtgaagtgtgatacataagtcatctcatttatgtaaacattgtgaggaaatagagcactggagagttaaggaactcactcgggtactcactgctggtaagtgatgcagccaggattcaacccagcccatcagatggcagcatctgcatctataaccattacactatttctttcttcaagctcccaatttgtgttctgaggagagactaaataaggaaaagaaaaaaatgtcttcccctatttagccaccagacctttgcccatgacttggaagtgtttgctgtggagtctcctatcttgtctttttttttttttttgagatggagtctcgctctgtctcccaggctggagtgcagtggcatgatctcggctcactgcaatctcctccttctgggcccaagcgattctcctgcctcattcttcccagtagctgggattataggcttccgccccaacgcctggctaatttttgtatttttagtagagacaaggtttctctattggtcaggctggtctggaactcctgacctcaagtgatcctccccctttggtatcccaaagtgctgggattacaggcgtgagccactgcacctagcctcctatcttgtcttcatgagtgttcagaattttgtggtgcactgatcagaagcccagaggaatctagacttcattgaggtgcaccttggtatttgaatcccactctctctttttgtttttattttttggtggagtgggagggtaggggcagacagagtctcactctgttgcccaggccaaagtgcagtgggcacagctcactgcagccttgacctcacaggcttaaatcatcctcccacctcagcctccggagtagctaggactataggtgcaccaccacacctggctaatttttgtattttttgtagaactgaggtttcgccatgttgcccaggctggtctcaaactcctgggctcaagcaatctgcccgcctcggcctcccaaagtgctgagattacagtcatgagctactgtgcccagcccaaattcctctttatcagcatcagatcatatcaatatcaattttataaagaccattaatatcaaccaggagaaggatgaaagcaaaatgttctaagagcaaagggactgctgattatcatcaagtgaccctatttcacaggaagaaaatgtggatgacattttatgaccaacagcattgtttatttaaccttggagcacaatagagaaaaacttttgactccaacccccagtccctgaaggcagaagtctgaaataccctctcaattctgtttatggccttaaagcatttgtcttagcctatattctccccaaagcagggcctgagacaaaggcttatgtgtgagtagtttaatttgctgtgctgatctcaggaagcaggagctatgcttgcagaaaatgaaatagggagtgcaaatataaggataagatattaactactactgaggacaactgattactcaagcatgggagaccttctgagagaccatataggatgcatttaaggactgtcttgctcaggggaagagagggagaaaaggttatccatcagtttccctctcccattggtcaagactggccctagggaacatttatgtttcctgtaaatgagtgctaagtggactcccacagacatcaggcatggcagctcaaagagactggaaggcagaaaatgaaagacatgagacgggcctgaggccaggcattttcaggttacatcttcatgaagatggtcactgctgcacaatggctggaataagaggtgagactgacaagatatgaagtgggacatgagaggtgtttgacagagtccaacccttgcaccattcaagcccacttaaaatgcccttgtgtaaatttaatttgtcatacggtcgtcttcagtatggcagttggctgcgttctctgcaaacacttgataatccaagcaaattagtgaaacaagttataacacttgctgctacagttgatcccaaagctgtaattgatattcattatctgcttcttctaccactcaatctagatttctcttattctcatctagcactttggctggcctggatttcttgcttggtggtgggactatgactgtccctgaagtccttatgattgccttacccttgatatgatcatgattactacaaatagcctgttcatcattaccactggatttgttcctgagctgacacttaactgagccttaccagaacattccaccatcatctgccagggtctgttttattgcagaagccatagaggtgatctgaatagagatataatgggcataaacaggcttgtgtcctctaagtctgttctgatggtgttaatctctgcagttctttctgggatgcaatattgcttctactgatgggggcagtgaagtgtctgggtaacttcaggggcttccacttggccctttctactacagactcttactcggctggatgtggtggctcacgcctgtaattccagcactttgggaggccgaggcgggcggatcacttgaggtcaggagttcgagatcagcctggccaatgtggtgaaaccccatctctactaacattagagactacaaaaattagccgtgtgtggtggtggcatgcctgtaattccagctactcaggaggctgaggcaggggaatcgcttgaacccaggaggtggaggttgcagtgagccgggatcaagccattgtactccagcctgggtgacagaacaagactctgtctcaaaaaaaaaaaagattcttactcctcaggtcagaaaaacaacacaagaattctaatatctgctaaatatatatctgctgatctctacttccagaaactggggaattaaccacagggtgggtccgtgaatgcatagaaccaatgtgaaatgcatttgggctagaagtcattttatcacttggctttcatcattcaaattcatcatctaaaaatggggggctgcatgcggtgcctcacacctgtaattccagcactttgggaggctgaggtgggtggatcatctgaggtgaggagttcaagactcatcctggcccacaatgcaaaacccgtctgcactaaaaatacaaacattagccaggcgtggtggcgggcacttgtagtcccagctactcgagaggctgaggcgggagaatcacttgaacccaggagatggaggttgcagtcagccgagattgtgccacttcactccagcctgggtgacggagcgagactccctgtcaataaataaataaataaataaataatgggggtgacagttcttatcacttacgggttttgtgaggattaaatggtatgtgtaaacaccaaaacagctgttgacacacagtaggtgttcaagaaatagtactgtgataggacatttttctacagaaatcctgactcccgctttggatgatttccttgaatcctgagttttttgaattccccaaattgtcctccatggtactacatagtcattaagaacaaagatttaggagccagatcattggggttttgaatcttggctctaccagtcactagctgttggatgttgggcaagtggtttaatctatctgtgattcaatttctttaatctataaaatgagaaaaattatatatctcacagtattgttttggtaattgagttattatatggaaaacacctagaattgcctggcacatagtaagcagaatgtaactgttagctattattattttattgttattttttgagatggggtctcactctgttgcccaggctggagtgcaatggtgagatcatggcttgctgcagcctcaacctcccacgcttaagtgatcctcccacctcagcctcccaagtagctgggactacaggtgcatgccaccacacccagctaatttttgtatatttgtagagacagggtttcaccatgttacccaggctggtcttgagtcctgggctcaaacaatccgcacactttggcctcccaaagtgctgggattacaggtgtgagccaccatgcctggcctattattattattaattggccaacactaccacctagaatttcttactagctggctttttttcccccatccgttgcttagctacatatatctcccctctcactcttctctttctccttccaaatcaaaaggttacagcctgctggtggccattctgacaggctgggaagcataggctccagttagaagccagaaacaggctcttcctctccttccaaatcatacctgggggactcagcttttctgggaaacgtaataatagaaacatttacccagcacttactcagagtattgtgctaaattctttccatggatgaactcttaattttcacaacaatcttaagaggtagatataaatattatgcccactttatagatagggaaaattacagtttcagagagacatgacaacttacctaaggtcacattctagaaattggtagagttgggatttgagcccaggtttgtgatattccctagcctattctaaccataggaatatggcattgcgtagtagtaaatttatgcactctagcatcagactgtgttcacatgctggactcacagctgtgacttactactgccttgcctacttaagtggcttctgaaaatatttggcctttccattggagatggagacaaatgtgggcagtaatgtttttaaatagagttttccccaatacccaacatagaatctgccgcagagcagatgcctcataaacattggctcagaaaataaatatattcacgacactattgtaggcacggtgtgggttacaaaggcagttgattgattacttatttattgcaaggaattggctgttttgctcgtcagggctggcttggcaagttaaaaatctgcagggcaaaccagcaggctggaaactcaggcaggagtggaagcagcaatccacagtaagtttcttccccaaggaagacttcagtttggtctttaggcctttacactgattggatgaggcacacccagattattgaagacagtctcttttattcaaaagtcaactgactgtacatgttaaccacatctacaaaataccatcacagcaacacctagattcgtgtttgattaataaagtaggtactatatcctagccatattgacacataaaactaaacttcatggtcagacttggtggctcacgcctgtaatcccagcattttgggaggccaagacagggggattgcttgagtccaggagttggagaccagcctgagcaacatggcaaaaccctgtcactacaaaaaatttaaaaaactagccaggtgaggtggcgggagcctgtaatcccagctacccaggaagcttgagccctggcagtcgaggttgcagtgagccaagatcgcgtcactgcactccagcctgagtaacagagtgagaccctgtctcaaaaacagacaatcaaaaaactaaactaaactaaccttcacttaattttttcaagttcgtttcatcagctataaaattgagatactgatccttacctccttacctcctatatgaaaatgcacattaggtagcagaactttgcatgaagcagagcaacagggcttaacaggtcttagcgctcagtctacaagaatcagtggggaggccgttgcaatgctttgacgagcagggtttggtttgaataagaatatatttttccaacatggatattttgtaatataagcacatgggtggcggggaaacagttcaaactggcaatcaatgaagaggttgtctcactctaggcctttccctcagaagtaatcgttgctgatagtttctgactatttttccagaattttgttctatatgcatgcacgctcaatgcctaatcacactcagccttttgtccccttgcaccttgctttttaaatttaatggtatgtcttggagaatgttccatttcagcatgtacaaatctacttcttttaaaaaatggctgcaaagggctgggcgtggtggctcacgtctgtaatcctggcactttgggaggccaaggcaggcggattgcctgagctcaggagtttgagaccagtctgggcaacacagtgaaaccccatcaatacaaaaaaaaaaaaaaattagccgggtgtggcagcgtgtgcctgtagtcccacctactcgggaggctgaggcaggagaattgcttgaacccaggaggcggaggttgtagtgagctgagatcacaccactgcactccagcctgggcgacagagcgagactctgtgtcttaaaaaaaattggctgcaaagtattccattgtgttgttaaatactaatttacttatttaataagttctcaactgatggtaatttaggttaccaacttttttgctattataaataatacaagggtaaacatctttgtacatacatacatgtttgcataatgtgtaagaagagatgtaagataaatttcgagaactggaaatactgcattaaagggtttgcacatttaaaatgtcaatgtatagtgccactttatctttcaaagagattgcattagtttatacccaaccaatagtatacgagagtgctgtttccccatgcccttatcaacactgccaccttggatttaaacctacttgttttcctttaaaataccaaaatgggcatttgaaaaaaagaggcatgatttgttccttggcaagagctatattttgtttgagtttgatctttctgatagcagacctaggccttgtgccctgtcctgtggtcccatctctgaggagctgggcagatgacctgaccacaggactcaatcaggatagcttcaggtcaccaggccaaagggtaggccagacccgggcctgtgcctagcctgatgatggaaatgcaaacaagccaagttctataatagaacacagagccattgccgggaaaccacaaattgctgacttgctcctgcaaacaatcaatattcagaagctgagtccaggaattctaacgatgcatcctgcacaaagggctgagtgggggaggatgtgcttttagaaaaaaacccagcaccagaagccctggtgctgcttcttagagggccatcaagaaggcccttggtcccatcaccacgccctctgacaatcccagaaccctaggctctcggagccagagtagatctgggaaattatcctgccctgagagagtccaggcaaagaatcaagggagttccaaggagcaagtgctcacttcggtgggatagtgcagggcagtggttagtggctacacttgggctcaaatcctgactcagtcaccagctttctgaccttggacacattgcttcacatctctcagcctcagttttctcatctatagaatgaagacaataacacctatctcagaaggtcattatgaagattcaactgaataattatgtttgtaaataatttagcatggcaactggctttttgtaaggtcgtaataaattattattaatattcattttttttttgagacagagtctccctctgtcacccaggttggagtgcagtggcactatctaggcttgaatcctcctgggttcaagtgattcttcggtctcagcctcccaagtagctgggactacaggcatacactagcacgcccagcaaattttttgtatttttagtagagatgtggtttcgccatgctggccaggctggtctcaaactcctgagctcaagtgatccactgaccttggcctctcaaagtgctaagattacaggtgtgagccaccatgcctggcctatattgttatgaataaacaataggatatcaggagtggggactaaggagaggccatttgcactagaccttgaagaatttggaagatttggtaattagaatgattaaagggatgaagttcctgtgcaaaggttctcttctcgtgtctctcctggcctgaccaggaggcatgctggggtctcagcagtagaccactgcagaggagggtgggtatatgtaggggataattccactgcagaacatgtcaaatagtccttgcttaaatggtatctacaatgacatttgcatctgtgttccaaattcatccccatatcctcctgtctcagcctcccaaagttctgagattacaggcatgagccactgtgcccagcctccatccacatttgaatgcaggctccatcagggcaggcccctttactgttaaaattctgcgtgatgatgaagggcacatgcctgcacggatttcctctactctgttccaccttagctgcttccctccctcagcaacgcctggcatccctactctggggtcgttacttagaggcaggatatggctccaatttagcaccattatccccaggaccagatagacagtaattggaatgaatcaatataacatacaaaactcttcataatctgggttcagtatatttttttagccattctcttagcctagactccagctagtctctagcatagactacatagaataatggtaacattgggctgggggtggtggctcatgcctgtaatctcagcactttgggaggctgaggtgggtggatcacttgaggccaggagtccaagacaagcctggccaacatggtgaaaccccgtctctactaaaaatacaaaaagtagccacgcatggtggtgcacgcctgtagtcccagctacttgggaggctggggcaggagaagagcttgaacccgggaggcggaggttgcagtgagccaagatcgtaccactgcactccagcctgggcaacagagtgagactctctctcacaaaacaaaaacaaaacaaaacaaaacaaaagaataatggtaatattgatcatcattcactgagtgtgtaccagtcattgttctagcactttacatgcattaattcaccgaatccttacagaaccctatcagttaagtattattattatctccattggtctggtgagaaaattagacccagaaaggtgaaacaaattcctgatagagcaaggacttgaagccaggtggtctgcctggaccctgaaccattcccagccactcctctctgttgcctctgctgcagagtggctttgctctttcaagtcgctgagcctctgctctgctgcaatgcccttccccaacttctggctctagcaaactctacttgccctttgaagcccagcccagatactgcatccctggccccacctgcttctccaaccaggggaaggaggttgtccctgtctccactttgttccccttacaagttgctcccagtcctggatggggaggtcgtctgaaacaggaccctgactcacgcctcagtatccccagccctcagcacagtgcttgtctgtgggtctttcatttgtgttttcaacaaacatgaacgagttgaatgagtaactccactgcttcacacacaaggaaacaaatccagagaggcagagggacctgtccatatcacacagccagtgagagaaagagctgaggccagactccagggctcctgacacccagccctgctctctctcacgccatcccaactgtgtgtggaatgtttggaaaagccttcatgcatctttgagcctttcttaattctgtcttacatttatttcatcccaaagaggacttctttgagaatgctggtgggagagtgaattttggctcgtcttgtctgaagtcatgaggcaacagggaaaaacagacaaacaatcctgagatgtgttttactgacccggtcctcaaaacagcagaacaaaacactccacactcaagctggggctgctagccagcagggcactgagtgaagctggacagaattcactagggatcctcctcccttttgcttaaaatggtggggactttggagaggaagtgttttctggttagtctgaattttctttttttttttcctttttttttgagatggagtcccactctgctccccaggctagagtgcagtggcacaatctcagctcactgcaacctccgcctcctgggttcaagcgattctcctgcctcagcctcccgagtagctgggattacaggcgtgcaccaccacacctggctaattttcatttttttagtagagatggggttttaccatgttggccaggctggtcttgaactcctgacctcaggtgatccatccgcctcagcctttcaaagtgctgggattacaggcttgagccactgtgcctggccctttttttttctttttctttctttttgacagggtcttgctctatcacccaggctggagtgcagtggcacaatcatggctcattcccacctcagcctcccaggcccaaatgatcctcccacctcacccgtgtagctgggaccacagctgtgcaccaccatgcctggataatttttaaattttttgtagagatgaggtctccctatgtcacccaggctgatctcaaactcctaagctcaagggatcctcctgcctctacctcccaaaatgctgggattacaggcttgagccactgtgcctggcccttttttttctttttctttctttttgacagggtcttgctctaccacccaggctggagtgcagtggcacaatcatggctcattcccacctcagcctcctgggcccaaatgatcctcccacctcacccgtgtagctgggaccacaggcgtgcaccaccatgcctggataatttttaaattttttgtggagatgaggtctccctatgtcacccaggctgatctcaaactcctaagctcaagggatcctcctgcctctacctcccaaaatgctgggattacaggcgtgagccaccacacctggccagtgatgattacttattcatgatgtcctttatggctggaatggccttcctcaccttgtctgcctaggaaacttttacttgtccatcataagggtgttctccttggcaaggcatctctgaccatcccgtttcccctcagtgtggtccctggttctctcagctctctcaccccttttacctttgcccctctaacacaatctgatttatgttgaaattgtttacttgcccatcttccctagtaggtgatgccagctaagaggataggctcaggtctgtgccctcagggcctaacagtgcctgccccatggagactcaggaactggcttctacatccctagatcaggagcactaggaggttgggagaagagaagctttgcagagctgggggctgcacagctcctgggagcacaaatcatatcatatgctattactttgtttgaatggagtccctggagctgtgcagcttggtagctctgggcctctgcctgagacagacttggtcttgaatcttagctctgccacattctggcttcactccttggaccatttaatctgtttaggcctcagtgtcttcacttgtcaatgactgttatgattgtgacctctcagattgttgtcaagattaaatgagatgatacacacaacgatcccgatggtgcctggccagtggtagttgtaacccggtaccggatttttaatgacaagattcataccagggctgagcatcagactatggggttgccttggtagtgtaagaggcagaaaggtaatgtgaagaggcagaaagaacagtgctctaggagacaagaaccctggatacaagggctgtgtgtgtgcacatgtgtgtgtgtactttctctgccctaccattgtggtccatcttcccctctgtcccaggcccatcctggttttgtactccatactgagctggaaaaatttgaggtagaggagcagctaggaacccatctagggtggccaggcctacccgtgcgctgggaagcaggagtgcccttgagcagtggcactgctttgggctggagccatgaggcctcctctgtggtgggggtcatggtttggagcatgggctatggagccaacagcctgggtgcaaaatccctcttagccccttcctcactgttctacctgtctgtgcctcagcttcctcactggattagttcactagggctgctgtaacaaagtgccacagcctgggtggctcaaacagcagaagtcatttcacatagttttggaggctaggagtgtgagatcaaggtgttggcccgattggtgtctagtgagggcctctctcctcggcttgcgggtggctggctttgtggttctatgttcgctattggtgaaataagggacagtgagctcagaaggaagtgaacttccagggtctaaatagcccaggtggaacagtgcacagacctgcagtgacactgcccagctggccaatactggactgtgtttcatcctagacaatttcaagacacaagtttgttttatgttctattaataccgtataagaataaacactagaaattgtaattgtaagacaccatcaattaagtctcactctgatctcagagatgctgaaatttgaaaaaaatatgtgttgtgtcttagaattgatgaaagacagtaggtcctgttttttagcttttggcagtgaggatccactgaggtggagggggggcaggccatgtgtctggggccgcagacgctggctgctgtccagtatcagcgtccacacaggaagtggaatggtgaagcaccatcccagtcaagtctcctcacttggaaaactggctaaatccacggctgggaaacagatctcaaaagctgcacaggtcccacttgagctttttggtgggccttgtaaaatgagccctttgcgtgggcatctgacagacctgggttcttgctctaaagctgctactcatcaaccaagaaattgaaagatgaaaggctccaaagtgcaaagaggggccaggcatggtggctcatgcctgtaatccctccgaggtgtgtggatcactggagcccaggagttcgagaccagcctgggcaatatggtgaaacctgatctctacaaaaaaaaaaaaaaaaacaaaaacaaaaagaagaagaagaaaattagccaggtgtggtggcacacaccagtagccccagctactctggaggctgaggtgggaggatcacttgagcccaggaggtcaaggcttcagtgagccatgatcatgatgccactgcactccagcctgggtgacagaatgagaccctgtctcaataagtaaacaaaagtgcaaagagggctttgccaaatgttgcatgcacaagatggtgtgtgtgtgagagacagggctgggagtcaggggtcccggggttcggttttggcttttccctccctggctgtggacttggccaggtccctctgcctctctggacctgtttcctcgtgggtaaagcagttgagttcagtcactcatccaacaaatgtgaactgggggccctttctgggtggtgtgctgagggctgggggtatacagggtgagatgtggtctccgctcccggagagctccgtccagtgtggagagcaaggagtgaagagagcagagaggaggcagccaggcattctccatgcactgtctcctttaatcctcatcatcaccttatgtgatgggtaacacaggcatttttattgccatttcagagatgcagtcatggagactgagaaagatggtgctgcttaccaaggcagggccagggtttggacctgttctgccttacctaaagccagttctctttcctcttcaccttggatggtcagtcagaagtttccttctagcacagcattctcagattctatgccaaaaaatagcctgtgagtgtcacattctgacaggcttctacaagatccccctcacttagcaaccaagtaagggagagcctctctctgctcaggaatggtggggccgggggtaggggtgcagggtagctcagcttgtctaacctggcttgtcttctaaggaggtggtaagcaccatgtttcaggggagtgagcctccccctcacaaaagctttccatctacact

SEQ ID NO: 2 - protein, 526 aa, Homo sapiens
MAALTIATGTGNWFSALALGVTLLKCLLIPTYHSTDFEVHRNWLAITHSLPISQWYYEATSEWTLDYPPFFAWFEYILSHVAKYFDQEMLNVHNLNYSSSRTLLFQRFSVIFMDVLFVYAVRECCKCIDGKKVGKELTEKPKFILSVLLLWNFGLLIVDHIHFQYNGFLFGLMLLSIARLFQKRHMEGAFLFAVLLHFKHIYLYVAPAYGVYLLRSYCFTASKPDGSIRWKSFSFVRVISLGLVVFLVSALSLGPFLALNQLPQVFSRLFPFKRGLCHAYWAPNFWALYNALDKVLSVIGLKLKFLDPNNIPKASMTSGLVQQFQHTVLPSVTPLATLICTLIAILPSIFCLWFKPQGPRGFLRCLTLCALSSFMFGWHVHEKAILLAILPMSLLSVGKAGDASIFLILTTTGHYSLFPLLFTAPELPIKILLMLLFTIYSISSLKTLFRKEKPLFNWMETFYLLGLGPLEVCCEFVYPFTSWKVKYPFIPLLLTSVYCAVGITYAWFKLYVSVLIDSAIGKTKKQ

SEQ ID NO: 3 - protein, 526 aa, Homo sapiens
MAALTIATGTGNWFSALALGVTLLKCLLIPTYHSTDFEVHRNWLAITHSLPISQWYYEATSEWTLDYPPFFAWFEYILSHVAKYFDQEMLNVHNLNYSSSRTLLFQRFSVIFMDVLFVYAVRECCKCIDGKKVGKELTEKPKFILSVLLLWNFGLLIVDHIHFQYNGFLFGLMLLSIARLFQKRHMEGAFLFAVLLHFKHIYLYVAPAYGVYLLRSYCFTASKPDGSIRWKSFSFVRVISLGLVVFLVSALSLGPFLALNQLPQVFSRLFPFKRGLCHAYWAPNFWALYNALDKVLSVIGLKLKFLDPNNIPKASMTSGLVQQFQHTVLPSVTPLATLICTLIAILPSIFCLWFKPQGPRGFLRCLTLCALSSFMFGWHVHEKAILLAILPMSLLSVGKAGDASIFLILTTTGHYSLFPLLFTAPELPIKILLMLLFTTYSISSLKTLFRKEKPLFNWMETFYLLGLGPLEVCCEFVYPFTSWKVKYPFIPLLLTSVYCAVGITYAWFKLYVSVLIDSAIGKTKKQ

SEQ ID NO: 4 - DNA, 474 bp, Homo sapiens
ggagccaagacgcccagttaaaaagtgcccaggtaggtccagcataattatttgcctggctggcgagttttggggccctaactttaagcttttttatgttgtcatatagcaggctggattgatttagataaaaacaacattcttcatttaaaaatatgcagagttctcctttttcagcagtgagtaggtcaaggccttggtgattttggaggacaactgcgactaaggaaccgacctgggcccgaaggactgataaagtttgtgatatatctgtaatgctagcaggaaagtcattagaaakacaatggaaggttgtgacagaagttgaaatgccaggtaggtcagagagatacagtcatgggggtcaggtgtggtatcaggaataatgtgggaggccggattgaagtctgggccaggaacaatggtaattatgggagactcaacaaagagtgagtatagctgaaggagctggag

SEQ ID NO: 5 - DNA, 601 bp, Homo sapiens
gtaacgatccagaagtttttttgtgcatacaaaatagcatttcatttatatttccttagcacaggattctaattatgctttatatcctcacaatggttagcataggctacaaataattatgcggttaatcagattccctgtttggcttgagcaagcacagcagtctgaacgatctcagacaattttaatagggaaaggtgagtttttttcagatgtgtttcagtgcttatggagggccagtggcttctcaccatggtgacaatccggttgctaggctgggcaagaccaagtgtggaagaayggtgctctgaacagttctactgaaacaaatggctgatgccactggcaactttcttcaagttctggttaatttcccatgaaataattgcttggcacacatctaagcagttcctttattcatgtttcttgtctgccaatagcagagtcaatcaatactgaaacatacagtttgaaccaagcatatgtgatgcctactgcacaatacactgaggttagtaacaaagggatgaaggggtacttcaccttccaggaggtgaaagggaatacaaattcacagcagacttccagaggccccaggcca

SEQ ID NO: 6 - DNA, 600 bp, Homo sapiens
tagcataggctacaaataattatgcggttaatcagattccctgtttggcttgagcaagcacagcagtctgaacgatctcagacaattttaatagggaaaggtgagtttttttcagatgtgtttcagtgcttatggagggccagtggcttctcaccatggtgacaatccggttgctaggctgggcaagaccaagtgtggaagaatggtgctctgaacagttctactgaaacaaatggctgatgccactggcaactttcttcaagttctggttaatttcccatgaaataattgcttggcacayatctaagcagttcctttattcatgtttcttgtctgccaatagcagagtcaatcaatactgaaacatacagtttgaaccaagcatatgtgatgcctactgcacaatacactgaggttagtaacaaagggatgaaggggtacttcaccttccaggaggtgaaagggaatacaaattcacagcagacttccagaggccccaggccaagcaggtagaaagtttccatccaattaaaaagaggtttttcttttctgaaggaaaaaagagagaaaagtctgattttagatgagtcatcctggttt

SEQ ID NO: 7 - DNA, 600 bp, Homo sapiens
ttgtgggattgggagctgcctagtggtggcagtcagggaaacaggacttggagtttcatttccaaccctatagctcatctcttggacaatgcccttcacctgctggtgtttggtgtgaattcagactaaagacattaaaagtttttgctttactttttaatttggaaactccaagctgctaaataaacccagacaagccacatactggcctgacaaatttgcttaatatatgttcagagcttaaatgatttctcaagattcaagaaaaatataaagaaagaggctattattaccatttatytatctattgggtttcaactatgcctaaactggacacttacatttaaaatggtttttttagcatggcatagtgaaaagatcatagattcaacatcataagattcaggtcctaactctaattcttgcgacctgtatgactttggacaagtcacaaacgttttgagccctagattcttcagatgttaagacagggaataagcctgttttgttggctcacatctgcaatcccagcactttgggaggctgaggtaggaagactgcttgtgcccagtagtttgagaccagcctaggcaacatggc

SEQ ID NO: 8 - DNA, 599 bp, Homo sapiens
acaaccagtcccagggaaataacacgaacaaagctgaaactcttccatcgaatagacccatctacagaaaaggaacattatcagtaaccaggaagatggaaaggaacattaaccataacctatatcatgtgctaaaactagaagtatcccacaatagtataccaatctattcatgatgtggcaagagcagagaaaagcgagaatgggtaaagggcttacaggattagcagctgagatatctgctgtgcttcataaaaccttcttatcaagcaagccaccaagtcaacacagtaagggaaaratcgaaaatgtttttgctattattaccaaaacttgcctggtttacttgcagtgaaacagtaggatcgcagcagatatacaccataagctggtgctacatagaggtagatatgcttgaaatgtaggagaacagcaaagagaaatgctccttccatatgccttttctaggagatttaaaagggaaatatcactttaaaattcagggtaaatgaaatgcaataatgtggtattctagattgaatcctggaacagaacaaggacattaaaggaaaaacgggtgaaattcaaatgcatcaata

SEQ ID NO: 9 - DNA, 600 bp, Homo sapiens
acctggcacctcaggcatctgataatactggctgaatggctgttggaggcagaggttcttaactgcctattaaggatgaggaccctgaggttcagaaacgttaaagtgatttgttccaaatcagacagcgccaggtctgaacctagccagctggggctaagtcaagtaacaactggcgaaacagaaagcttagcaaaggcaggatagcgacaaacacgacctaaagttttctcttcatacccagggatatccacacctttctctcccgccctgaccgaccgcggggcctccccgcccagcmcctggccgtgcgagtcccttactatgtggggatgagaaggcatttgagaagagtcaccccgagcgccaaagccgaaaaccaattgccagtacccgtggcaattgtgagcgccgccattgctgcggcaccgcacgcttcccaccaacttgatccacatccgggatcccgcgcatgcggagaaagccctctgaagccgtgcccgctagctgcgcgcatgcggcgagcggcgcagccagtccggggactgcagtcagctatttaaacctcccgcccaccttttctttatacccgtgtctcac

SEQ ID NO: 10 - DNA, 625 bp, Homo sapiens
ttccgctcaagcaggaggtgagccaagagcctcgctcagagttctaaaaacacaatgtttctcacctctgtatctttgcacatgccattctctagctggatttaatactccggtgcaagcgtccaggttgggagcccccgcagttctctgaggtacttttatctactgtaactctagggcaacaatcctggcttctccactagtgcgtgaacgtctctagcgttaagtttttttctcacctaagtatccccaatactacacagtgatgacccagcgcaaacgaccactctttcaacgcaagcatgaactagctgataaaacttaaaagtggctacaaacgcctgcgcaggagcccttccsgcccggggtgagacgcgggtctaaagaaaaggtgggcgggaggtttaaatagctgactgcagtccccggactggctgcgccgctcgccgcatgcgcgcagctagcgggcacggcttcagagggctttctccgcatgcgcgggatcccggatgtggatcaagttggtgggaagcgtgcggtgccgcagcaatggcggcgctcacaattgccacgggtactggcaattggttttcggctttggcgctcggggtgactcttctcaaat

SEQ ID NO: 11 - DNA, 884 bp, Homo sapiens
  misc_feature (558)..(578): Represents location of insertion/deletion.
ctgaaactgtaattttccctatctataaagtgggcataatatttatatctacctcttaagattgttgtgaaaattaagagttcatccatggaaagaatttagcacaatactctgagtaagtgctgggtaaatgtttctattattacgtttcccagaaaagctgagtcccccaggtatgatttggaaggagaggaagagcctgtttctggcttctaactggagcctatgcttcccagcctgtcagaatggccaccagcaggctgtaaccttttgatttggaaggagaaagagaagagtgagaggggagatatatgtagctaagcaacggatgggggaaaaaaagccagctagtaagaaattctaggtggtagtgttggccaattaataataataataggccaggcatggtggtagtgttggccaattaataataataataggccaggcatggtggctcacacctgtaatcccagcactttgggaggccaaagtgtgcggattgtttgagcccaggactcaagaccagcctgggtaacatggtraaaccctgtctctacaaatatacaaaaattagctgggtgtggtggcatgcacctgtagtcccagctacttgggaggctgaggtgggaggatcacttaagcgtgggaggttgaggctgcagcaagccatgatctcaccattgcactccagcctgggcaacagagttgagaccccatctcaaaaaataacaataaaataataatagctaacagttacattctgcttactatgtgccaggcaattctaggtgttttccatataataactcaattaccaaaacaatactgtgagatatataatttttctcattttatagattaaagaaattgaatcacagatagat